(19) United States
(12) Patent Application Publication
(10) Pub. No.: US 2004/0067507 A1
(43) Pub. Date: Apr. 8, 2004

(54) Liver inflammation predictive genes

(76) Inventors: Timothy D. Nolan, West Palm Beach, FL (US); Usha Sankar, Port Washington, NY (US); Larry D. Kier, Santa Fe, NM (US); Maher Derbel, Cambridge, MA (US)

Correspondence Address: ROBERTS ABOKHAIR & MARDULA, SUITE 1000, 11800 SUNRISE VALLEY DRIVE, RESTON, VA, 20191, US

(21) Appl. No.: 10/434,799

(22) Filed: May 9, 2003

Related U.S. Application Data
(60) Provisional application No. 60/379,831, filed on May 10, 2002.

(51) Int. Cl. C12Q 1/68; G06F 19/00; G01N 33/48; G01N 33/50

(52) U.S. Cl. 435/6; 702/20

(57) ABSTRACT

The invention provides toxicity predictive genes that can be used to predict toxicity in response to one more agents. The invention provides for a method of predicting the liver toxicity In Vivo or In Vitro to an agent. The method comprises obtaining a biological sample from an individual, cell culture or explant treated with the agent. The expression of one or more liver toxicity predictive genes in the sample is measured, wherein the genes are selected from a group consisting of partial gene sequences of genes identified as responsive to agents causing liver inflammation. The process generates a test expression profile. The test expression profile is used with a set of reference expression profiles in a Predictive Model to determine whether the agent will induce liver toxicity in the individual.

### CROSS REFERENCE TO OTHER PATENT APPLICATIONS

This application claims the benefit of U.S. Provisional application No. 60/379,831 and filed May 10, 1902, which is incorporated herein by reference in its entirety.

### REFERENCE TO A SEQUENCE LISTING AND TABLES

Description of Accompanying CD-ROM (37 C.F.R. §§ 1.52 & 1.58): Tables 26, 28, 29, and 30 referred to herein are filed herewith on CD-ROM in accordance with 37 C.F.R. §§ 1.52 and 1.58. Two identical copies (marked “Copy 1” and “Copy 2”) of said CD-ROM, both of which contain Tables 26, 28, 29, and 30, are submitted herewith, for a total of two CD-ROM discs submitted. Table 26 is recorded on said CD-ROM discs as “Table26.txt” created Apr. 25, 2002 size 288,877 bytes. Table 28 is recorded on said CD-ROM discs as “Table28.txt” created on May 6, 2002, size 634,567 bytes. Table 29 is recorded on said CD-ROM discs as “Table29.txt” created on May 6, 2002, size 444,079 bytes. Table 30 is recorded on said CD-ROM discs as “Table3O.txt” created on May 6, 2002, size 399,825 bytes.

The contents of the files contained on the CD-ROM discs submitted with this application are hereby incorporated by reference into the specification.

### BACKGROUND

This invention is in the field of toxicology. More specifically, it relates to liver inflammation predictive genes and the methods of using such genes to predict liver inflammation.

Molecular biology and genomics technologies have potential to create dramatic advances and improvements for the science of toxicology as for other biological sciences. See, for example, MacGregor, et al. Fund. Appl. Tox. 26:156-173, 1995; Rodi et al., Tox. Pathology 27:107-110, 1999; Cunningham et al., Ann. N.Y. Acad. Sci. 919: 52-67, 2000; Pritchard et al., Proc. Natl. Acad. Sci. USA 98:13266-13271, 2001; and Fielden and Zacharewski, Tox. Sciences 60: 6-10, 2001. These technologies provide massive amounts of parallel information for processes and events occurring at the molecular level. This level of information is in dramatic contrast to conventional safety assessment toxicology that, to a large extent, currently relies on subjective evaluation (e.g., in-life observations of behavior, observations of gross abnormalities at necropsy and histopathological examination of stained tissue slides using a microscope). These current methodologies may be largely subjective and in some cases such as histopathological evaluation, they require someone with a high degree of training, experience and skill to make competent evaluations. Furthermore, many of the methodologies require access to organs and tissues that necessitates either killing laboratory animals or surgery to obtain tissue specimens.

Recently, there have been some initial efforts to apply molecular biology and genomics technologies to toxicology. Some efforts have involved application of gene expression measurements. See, for example, U.S. Pat. No. 6,228,589 and WO 01/05804. Analysis of the data has yielded interesting observations of gene expressions that appear to correlate with some toxic effects or mechanisms. See, for example, Mueller et al. Environmental Health Perspectives 106(5): 277-230 (1998). However, there has been very little published work in toxicology so far that applies rigorous analytical and statistical techniques to the massive amounts of data available from genomics technologies. The observations, so far, have tended to be phenomenological and focused on individual gene responses rather than determining the generally applicable capabilities of patterns of gene expression to predict toxic effects (see, for example, studies of gene expression altered by exposure to liver toxicants in Bartosiewicz et al., Environ health Perspectives 109:71-74, 2001; Huang et al., Tox. Sciences 63: 196-207, 2001). Even in the larger field of biological sciences, these types of analyses are just beginning to be evidenced in the literature (e.g., Golub et al., Science 286: 531-537,1999).

Recently some work has been published that attempts to correlate gene expression profiles with the mechanism of toxicity of various hepatotoxins. See for example, Waring et al. Tox. and Appl. Pharm. 175:28-42 (2001). However there has been limited success thus far in the attempts to predict toxicity of compounds based on the gene expression profiles elicited upon treatment.

What is needed are genes and predictive models, which are capable of predicting toxicity response.

### SUMMARY

The invention provides liver inflammation predictive genes and predictive models which are useful to predict toxic responses to one or more agents.

One aspect of the present invention provides methods of predicting liver toxicity to an agent. A biological sample is obtained from an individual treated with the agent. Alternatively, a biological sample is obtained from an individual and treated with the agent. In vitro cultured cells or explants may also be treated with the agent. A gene expression profile on one or more of the liver inflammation predictive genes disclosed herein is obtained from the biological sample or in vitro cultured cells or explants used. The gene expression profile from the biological sample or cells treated with the agent is used in a predictive model to predict whether the agent will induce liver inflammation in the individual or would be predicted to produce liver toxicity following in vivo exposure.

In another aspect, the invention provides methods for determining the presence or absence of a no-observable effect level (NOEL) of an agent in an individual. A biological sample is obtained from individuals treated with the agent at different dose levels. Alternatively, a biological sample is obtained from In vitro cultured cells or explants treated in vitro at different dose levels. A gene expression profile of a set of liver inflammation predictive genes from the samples, cultured cells or explants is obtained. The gene expression profile from the biological sample or cells treated with the agent are used in a predictive model to predict at which dose levels the agent will induce liver inflammation in the individual or in vitro. In one embodiment, the predictive model utilizes sets of liver inflammation predictive gene(s) selected from one of the various liver inflammation predictive gene sets disclosed herein (i.e., Combination 5, 4, 3, 2, or 1), wherein the sets comprise one or more genes therefrom.

In another aspect, the invention provides methods of identifying a liver inflammation predictive gene. One method comprises providing a set of candidate toxicity predictive genes; evaluating said genes for their predictive performance with at least one training and test set of data in a Predictive Model to identify genes which are predictive of liver inflammation; and testing the performance of predictive genes for their ability to predict liver inflammation for: (i) different test sets of data, (ii) comparison of prediction for accurate versus random classification, and (iii) prediction using test data external to the data used to derive the predictive genes.

In another aspect, the invention provides a computer-based method for mining genes predictive for liver inflammation by: collecting expression levels of a plurality of candidate toxicity predictive genes in a multiplicity of samples; optionally storing the expression levels as a database on an electronic medium; defining a group of samples to be a training set; defining another group of samples to be a test set; optionally generating additional training and test sets; and selecting a set of genes which are predictive of liver inflammation based on evaluating the training set and the test set in a Predictive Model.

In another aspect, the invention provides a computer program product for predicting liver inflammation, which includes a set of liver inflammation predictive genes derived from mining a database having a plurality of gene expression profiles indicative of toxicity. In one embodiment, the set of liver inflammation predictive genes includes at least one predictive gene from combination 5, 4, 3, 2, or 1 list.

In another aspect, the invention provides a library of expression profiles of liver inflammation predictive genes produced by the methods disclosed herein.

In another aspect, the invention provides an integrated system for predicting liver inflammation including equipment capable of measuring gene expression profiles of liver inflammation predictive genes from biological samples exposed to a test agent, operably linked to a computer system capable of implementing a predictive model.

### BRIEF DESCRIPTION OF THE DRAWINGS

FIG. 1 is a flow diagram illustrating one embodiment of the present invention for identification of predictive genes.

FIG. 2 is a flow diagram illustrating one embodiment of the present invention for evaluating performance of liver inflammation predictive genes.

FIG. 3 is a flow diagram illustrating one embodiment of the present invention for predicting toxicity of liver inflammation predictive genes.

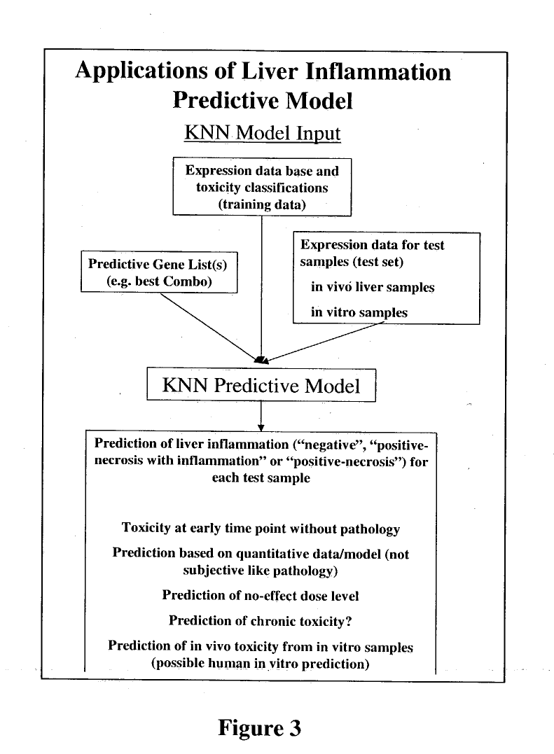

### BRIEF DESCRIPTION OF THE TABLES

Table 1 lists compounds, dose levels, liver pathology and abbreviations in the database in accordance with one embodiment of the present invention.

Table 2 lists the distribution of compounds in individual training and test sets for 24 hour liver data in accordance with one embodiment of the present invention.

Table 3 lists the genes whose expression at 24 hour directly correlates with liver inflammation at 72 hour, ranked by Pearson correlation coefficient in accordance with one embodiment of the present invention.

Table 4 lists the genes whose expression at 24 hour inversely correlates with liver inflammation at 72 hour, ranked by Spearman correlation coefficient in accordance with one embodiment of the present invention.

Table 5 lists the predictive genes for 24 hour expression data in accordance with one embodiment of the present invention.

Table 6 lists the randomly selected gene subsets from 24 hour Combo AII gene set in accordance with one embodiment of the present invention.

Table 7 lists the randomly selected gene subsets from 24 hour Combos 5, 3, 2 combined in accordance with one embodiment of the present invention

Table 8 lists the randomly selected gene subsets from 24 hour all excluding predictive genes (i.e,. excluding Combo AII genes) in accordance with one embodiment of the present invention.

Table 9 lists the liver inflammation individual sample prediction values for 24 hour data predictive genes (combined list and subsets) in accordance with one embodiment of the present invention.

Table 10 lists the liver inflammation compound-dose prediction values for 24 hour data predictive genes (combined list and subsets) in accordance with one embodiment of the present invention.

Table 11 lists the liver inflammation compound prediction values for 24 hour data predictive genes (combined list and subsets) in accordance with one embodiment of the present invention.

Table 12 lists the individual gene predictions for Combo 3 in accordance with one embodiment of the present invention.

Table 13 lists the individual gene predictions for Combo 2 in accordance with one embodiment of the present invention.

Table 14 lists the comparison of predictivity for correct liver inflammation classification and random classification using Combo gene sets and random subsets and 24 hour data in accordance with one embodiment of the present invention.

Table 15 lists the distribution of compounds in individual training and test sets for 6 hour liver data in accordance with one embodiment of the present invention.

Table 16 lists the genes whose expression at 6 hours directly correlates with liver inflammation at 72 hours, ranked by Pearson correlation coefficient in accordance with one embodiment of the present invention.

Table 17 lists the genes whose expression at 6 hours inversely correlates with liver inflammation at 72 hours, ranked by Spearman correlation coefficient in accordance with one embodiment of the present invention.

Table 18 lists genes whose expression at 6 hours is predictive of liver inflammation at −72 hours in accordance with one embodiment of the present invention.

Table 19 lists the comparison of predictivity for correct liver inflammation classification and random classification using combo gene sets and 6 hour data in accordance with one embodiment of the present invention.

Table 20 lists the distribution of compounds in individual training and test sets for 72 hour liver data in accordance with one embodiment of the present invention.

Table 21 lists genes whose expression at 72 hours directly correlates with liver inflammation at 72 hours, ranked by Pearson correlation coefficient in accordance with one embodiment of the present invention.

Table 22 lists genes whose expression at 72 hours inversely correlates with liver inflammation at 72 hours, ranked by Spearman correlation coefficient in accordance with one embodiment of the present invention.

Table 23 lists genes whose expression at 72 hours is predictive of liver inflammation at 72 hours in accordance with one embodiment of the present invention.

Table 24 lists comparison of predictivity for correct liver inflammation classification and random classification using combo gene sets 72 hour data in accordance with one embodiment of the present invention.

Table 25 lists the RCT genes (ESTs) predictive for liver inflammation at 72 hours: best homology matches in accordance with one embodiment of the present invention.

Table 26 lists the genes predictive for liver inflammation, sequences, and accession numbers in accordance with one embodiment of the present invention.

Table 27 lists the liver inflammation predictive genes whose protein products are known to be secreted. The genes are from the table listing all the inflammation predictive genes at the three time points 6, 24, and 72 hours in accordance with one embodiment of the present invention.

Table 28 lists the expression data for the 6 hour timepoint in accordance with one embodiment of the present invention.

Table 29 lists the expression data for the 24 hour timepoint in accordance with one embodiment of the present invention.

Table 30 lists the expression data for the 72 hour timepoint in accordance with one embodiment of the present invention.

### DETAILED DESCRIPTION

One embodiment of the present invention relates to methods of predicting whether an agent or other stimulus will or is capable of inducing liver inflammation using predictive molecular toxicology analysis. Another embodiment of the present invention provides methods of predicting liver inflammation which comprise analyzing gene and/or protein expression across a number of liver inflammation biomarkers disclosed herein for patterns of expression that are predictive of liver inflammation in the recipient organism. This type of toxicity is significant as a toxic effect of many chemical agents and is a significant component of adverse reactions to pharmaceuticals and drugs (see, for example, Treinen-Moslen, M. in Casarett and Doull's Toxicology: The Basic Science of Poisons Sixth Edition (C.D. Klaasen, ed.) Chp. 13., McGraw-Hill, New York, 2001). Adverse drug reactions are very often unpredictable, and may occur through acute exposure to the chemical agent or drug or through chronic exposures. For many drugs and chemical agents, inflammatory responses are implicated in amplifying or extenuating the initial toxic damage that occurs in the liver (see, for example, Treinen-Moslen, M., ibid.)

Another embodiment of the present invention provides that modulated transcriptional regulation of relatively small sets of certain genes in response to a test agent can accurately predict the occurrence of liver inflammation observed at later time points.

In yet another embodiment, the predictive model utilizes gene expression profiles from sets of liver inflammation predictive gene(s) selected from one of the various-liver inflammation predictive gene sets disclosed herein (i.e., Combination 5, 4, 3, 2, or 1), wherein the sets comprise one or more genes there from.

In still another embodiment, the predictive genes and models may be used to identify and evaluate various in vitro systems that can be used to accurately predict in vivo toxicity and to use the identified in vitro systems to accurately predict in vivo toxicity.

Provided herein are multiple sets of liver inflammation biomarkers which are useful in the practice of the liver inflammation prediction methods of the invention. In particular, applicants have identified 415 liver inflammation biomarkers which demonstrate utility in predicting liver inflammation. These biomarkers have been thoroughly characterized for their predictive performance, individually as well as in various combinations or subsets thereof. In addition, various optimized subsets of the liver inflammation biomarkers of the invention are disclosed. These sets have also been thoroughly characterized for predictive performance using the methods of the invention. Among the subsets of liver inflammation genes provided herein are several which demonstrate prediction accuracies in the vicinity of about 85%.

Other embodiments of the present invention are further described by way of the experimental examples provided herein. These examples demonstrate that small sets of genes (i.e., in some instances, as few as 1 biomarker gene) may be used to accurately predict liver inflammation. For example, as further described in the Examples, analysis of mRNA expression of only a few genes can provide an indication of whether a test agent will or will not induce liver inflammation.

The predictive capacity of the methods of the invention have been verified by comparisons with random classifications. Moreover, the methods of the invention are capable of distinguishing between agent dose levels that induce toxicity (typically higher doses) and those doses that are non-toxic. This latter feature is an important component of meaningful toxicological evaluation.

General Techniques: The several embodiments of the present invention employ, unless otherwise indicated, conventional techniques of molecular biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6th edition (2001).

Definitions: Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

“Toxic” or “toxicity” refers to the result of an agent causing adverse effects, usually by a xenobiotic agent administered at a sufficiently high dose level to cause the adverse effects.

The term “liver inflammation” refers to an inflammatory response of the liver that can be initiated by physical injury, infection, or local immune response and can include local accumulation of fluid, plasma proteins and white blood cells, as well as migration and infiltration of neutrophils, lymphocytes, and other cells of the immune system into regions of damaged liver.

As used herein, the terms “liver inflammation biomarker” and “liver inflammation predictive gene” are used interchangeably and refer to a gene whose expression, measured at the RNA or protein level can predict the likelihood of a liver inflammation response.

A “toxicological response” refers to a cellular, tissue, organ or system level response to exposure to an agent. At the molecular level, this can include, but is not limited to, the differential expression of genes encompassing both the up- and down-regulation of expression of such genes at the RNA and/or protein level; the up- or down-regulation of expression of genes which encode proteins associated with response to and mitigation of damage, the repair or regulation of cell damage; or changes in gene expression due to changes in populations of cells in the tissue or organ affected in response to toxic damage.

An “agent” or “compound” is any element to which an individual can be exposed and can include, without limitation, drugs, pharmaceutical compounds, household chemicals, industrial chemicals, environmental chemicals, other chemicals, and physical elements such as electromagnetic radiation.

The term “biological sample” as used herein refers to substances obtained from an individual. The samples may comprise cells, tissue, parts of tissues, organs, parts of organs, or fluids (e.g., blood, urine or serum). Biological samples include, but are not limited to, those of eukaryotic, mammalian or human origin.

“Sample” is defined for the purposes of prediction as a biological sample and the gene expression data for that sample. Each sample may come from an individual animal. A toxicity classification may also be associated with the sample.

“Gene expression” as used herein refers to the relative levels of expression and/or pattern of expression of a gene. The expression of a gene may be measured at the DNA, cDNA, RNA, mRNA, protein level or combinations thereof.

“Gene expression profile” refers to the levels of expression of multiple different genes measured for the same sample. Gene expression profiles may be measured in a sample, such as samples comprising a variety of cell types, different tissues, different organs, or fluids (e.g., blood, urine, spinal fluid, sweat, saliva or serum) by various methods including but not limited to microarray technologies and quantitative and semi-quantitative RT-PCR (e.g., Taqman™) techniques, as well as techniques for measuring expression of proteins.

“Individual” refers to a vertebrate, including, but not limited to, a human, non-human primate, mouse, hamster, guinea pig, rabbit, cattle, sheep, pig, chicken, and dog.

As used herein, the terms “hybridize”, “hybridizing”, “hybridizes” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees Celsius and temperatures for washing in 0.1×SSC/0.1% SDS are above 55 degrees Celsius, and preferably to stringent hybridization conditions. The hybridization of nucleic acids can depend upon various factors such as their degree of complementarity as well as the stringency of the hybridization reaction conditions. Stringent conditions can be used to identify nucleic acid duplexes with a high degree of complementarity. Means for adjusting the stringency of a hybridization reaction are well-known to those of skill in the art. See, for example, Sambrook, et al., “Molecular Cloning: A Laboratory Manual,” Second Edition, Cold Spring Harbor Laboratory Press, 1989; Ausubel, et al., “Current Protocols In Molecular Biology,” John Wiley & Sons, 1996 and periodic updates; and Hames et al., “Nucleic Acid Hybridization: A Practical Approach,” IRL Press, Ltd., 1985. In general, conditions that increase stringency (i.e., select for the formation of more closely matched duplexes) include higher temperature, lower ionic strength and presence or absence of solvents; lower stringency is favored by lower temperature, higher ionic strength, and lower or higher concentrations of solvents.

In the context of amino acid sequence comparisons, the term “identity” is used to express the percentage of amino acid residues at the same relative position which are the same. Also in this context, the term “homology” is used to express the percentage of amino acid residues at the same relative positions which are either identical or are similar, using the conserved amino acid criteria of BLAST analysis, as is generally understood in the art. Further details regarding amino acid substitutions, which are considered conservative under such criteria, are discussed below.

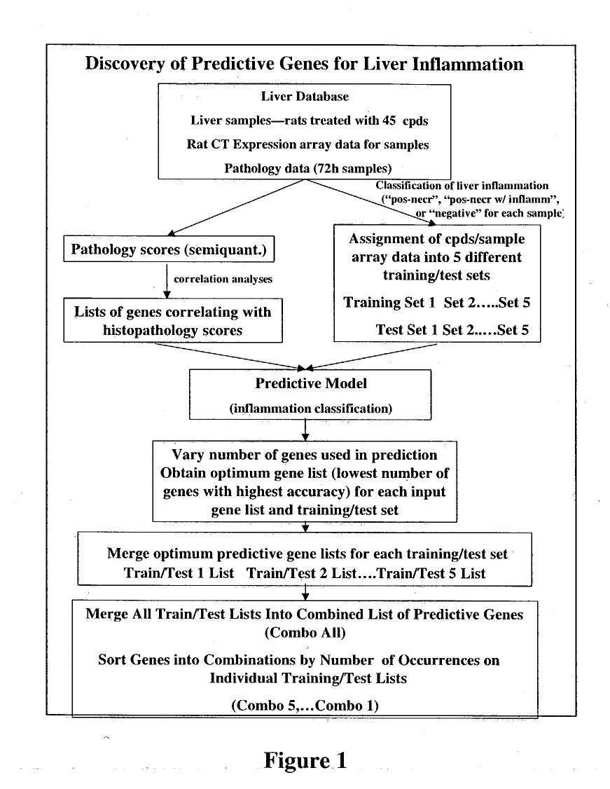

Identification of Liver Inflammation Biomarkers: Generation of Toxicology Gene Expression Databases: The liver inflammation biomarkers described herein were initially identified utilizing a database generated from large numbers of in vivo experiments, wherein the differential expression of approximately 700 rat genes, measured at various time points, in response to multiple toxic compounds inducing various specific toxic responses, as visualized through microscopic histopathological analysis, was quantified, as described in pending United States patent application filed Jan. 29, 2002 (Ser. No. 10/060,893). This quantitative gene expression data, as well as corresponding histopathological information, was then subjected to an analytical approach specifically designed to identify genes which not only correlated with the observed histopathology, but also demonstrated an ability to be used in a model capable of accurately predicting the occurrence of the toxic response associated with the observed histopathology. A detailed description of this identification process is presented in the Examples. A flow diagram illustrating how the liver inflammation biomarkers of one embodiment of the present invention were identified is illustrated in FIG. 1.

In addition to the database described and utilized herein, other toxicology gene expression databases may be generated, and used to identify additional liver toxicity biomarkers, which may also be employed in the practice of the liver inflammation prediction methods of the invention. Such databases may be generated with test compounds capable of inducing various pathologies indicative of a toxic response in the liver and/or other organs or systems, over different time periods and under different administration and/or dosing conditions, including without limitation hepatocellular necrosis, regenerative proliferation, neoplasia, apoptosis, fibrosis, and cirrhosis. An example of compounds, dose levels, liver toxicity classifications and histopathology scores used in the Examples which follow are provided in Table 1. The compounds and dose levels are abbreviated in the Abbreviation Column. The Inflammation Score relates the histopathology liver inflammation, a score of “2” or higher indicates histopathology of increasing severity.

Such databases may be generated using organisms other than the rat, including without limitation, animals of canine, murine, or non-human primate species. In addition, such databases may incorporate data derived from human clinical trials and post-approval human clinical experiences. Various methods for detecting and quantitating the expression of genes and/or proteins in response to toxic stimuli may be employed in the generation of such databases, as are generally known in the art. For example, microarrays comprising multiple cDNAs or oligonucleotide probes capable of hybridizing to corresponding transcripts of genes of interest may be used to generate gene expression profiles. Additionally, a number of other methods for detecting and quantitating the expression of gene transcripts are known in the art and may be employed, including without limitation, RT-PCR techniques such as TaqMan®), RNAse protection, branched chain, etc.

Databases comprising quantitative gene expression information preferably include qualitative and quantitative and/or semi-quantitative information respecting the observed toxicological responses and other conventional toxicology endpoints, such as for example, body and organ weights, serum chemistry and histopathology observations, histopathology scores and/or similar parameters.

Identification of Correlating Genes: For the purpose of identifying candidate predictive genes, the database preferably includes histopathology scores for each animal which has been exposed to one or more agent(s). These scores can be assigned based on actual histopathology observations for the tissue and animal or on the basis of effects observed for other animals treated with the same agent and dose level. The scores are numerical scores that reflect the occurrence and severity of histopathological changes. These scores can be adjusted to have similar range to gene expression changes. For example, a score of 1 could be assigned to samples with no changes and scores of 2-8 assigned to increasingly severe changes. Because the scores are numerical, they are suitable for use with a variety of statistical correlation and similarity measures.

An example of a histopathology scoring system is provided in Example 1. Referring now to FIG. 1, histopathology scores may be utilized to identify genes which correlate with the observed toxicological response, using any number of statistical correlation and similarity analysis techniques, including without limitation those correlation or similarity measures described or employed in Example 1 (e.g., Pearson, Spearman, change, smooth, distance etc.). Such correlating genes may be used as predictive gene candidates. Examples of genes whose expression at 24 hours after treatment correlates with histopathology observed at 72 h are detailed in Tables 3 and 4. In one embodiment, the correlating gene lists as well as the entire array gene list are used as input gene lists in the GeneSpring™ (Version 4.1, Silicon Genetics, Redwood City, Calif.) Predict Parameter Values tool (otherwise known hereafter as “Predictive Model”).

Class Prediction and Classification: Statistical analysis of the database of gene expression profiles can be affected by utilizing commercially available software programs. In one embodiment, GeneSpring™ is used. Other software programs which can be used for statistical analysis are SAS software packages (SAS Institute Inc., Cary, N.C.) and S-PLUS® software (Insightful Corporation, Seattle, Wash.).

Using GeneSpring™ software, class predictions can be made from the genes in the database, as detailed in Example 1, using one or more training and test sets. In one embodiment, five training sets and five test sets are obtained, as shown in Example 1 (Table 2). Liver toxicological classifications are entered for the samples in each training and test set. Compounds that did not elicit histopathology (score=1) are identified as negative for training and test sets. Compounds that elicit histopathology (score of 2 or greater) are identified as positive for training and test sets. Compounds denoted with Low indicates low dose of the compound is administered. Compounds denoted with High, indicates high dose of the compound is administered. Compound abbreviations in Table 2 are defined in Table 1. Toxicological classifications can be defined by the presence or the absence of various pathologies. In yet another embodiment, toxicity observed as inflammation is defined as three classifications (i.e. liver necrosis, liver necrosis with inflammation, or no histopathology (negative)) observed 72 hours after treatment with an agent. In another embodiment, toxicity observed as inflammation is defined as two classifications (i.e. liver inflammation or no inflammation) observed 72 hours after treatment with an agent. However, toxicity can manifest in other liver pathologies such as regenerative proliferation, neoplasia, apoptosis, fibrosis, and cirrhosis. More complex (four or more) classifications can be used in defining multiple pathologies.

Once the training sets have been selected, then predicted classifications of the test set samples are obtained by using k-nearest neighbor (or knn) voting procedure. The class in which each of the knn is determined and the test sample is assigned to the class with the largest representation after adjusting for the proportion of classifications in the training set. In one embodiment, adjustments are made to account for different proportions of classes in the training set.

Toxicity can also be observed at various time points after exposure to an agent and is not limited to only 72 hour after treatment. A skilled toxicologist can determine the optimal time after exposure to an agent to observe pathology by either what has been disclosed in the art or a stepwise experimentation with time increments, for example 2, 4, 6, 12, 18, 24, 36, 48 hours post-exposure or even longer time increments, for example, days, weeks, or months after exposure to the agent.

Identification of Predictive Genes: Referring now to FIG. 1, a description of the process used to identify liver inflammation predictive genes in one embodiment of the present invention is illustrated. According to this embodiment of the present invention, the process is run independently for each time point.

The number of input genes that are to be used in the Predictive Model can be varied, for example 50, 40, 30, 20, 10, 5, 2, or 1 gene(s) can be used. In one embodiment, at least 50 genes are used.

A gene list is generated comparing high predictive accuracy to the number of genes used. In one embodiment, optimum gene lists for all input gene lists are combined for each training and test set and then these combined lists for all five training and test sets are merged to create an aggregate list of predictive genes. The aggregate list can then be subdivided to smaller lists of genes based on the number of times that the genes occurred on the predictive gene lists for an individual training or test set. The resulting gene lists are designated herein as Combo 5, 4, 3, 2, or 1 lists. The genes that were predictive in all 5 training and test sets are designated as Combo 5 and the genes that were predictive in 4 of 5 training and test sets are designated as Combo 4 and so forth. Table 26 presents gene names, accession numbers and sequence information for the liver inflammation predictive genes found by analysis of the database in the manner described above in accordance with one embodiment of the present invention. Each of these genes has been demonstrated to contribute to predictive performance for at least one input gene list and training/test set and one time point. Table 25 lists homologous genes for the RCT sequences that were identified by BLAST search using the GeneBank NR database as the target database. Referring now to Table 25, homologies are given from Blast searches using Phase 1/RCT sequence as the query sequence and GeneBank NR database as the target sequence database in accordance with one embodiment of the present invention. The best Blast homology sequence observed is given. In general, no significant homology indicates that no Blast match was observed with a BIT score>100.

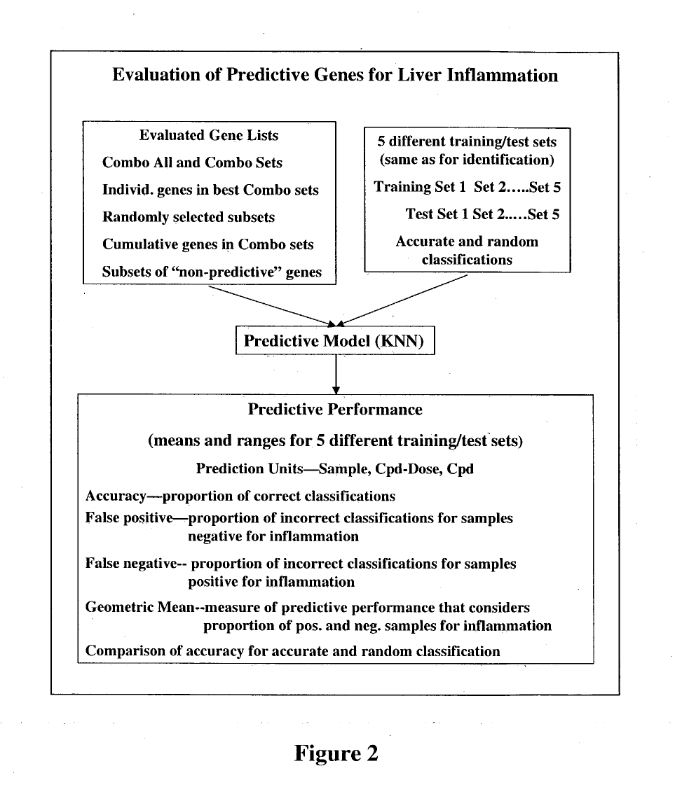

Evaluation of Predictive Genes for Liver Inflammation: The predictive genes are evaluated for predictive performance as illustrated in FIG. 2. For each gene list prediction, a table of data is generated using the Predictive Model which includes: the test set containing information about the actual call (i.e., negative, necrosis with inflammation, necrosis), the predicted call (i.e., negative, necrosis with inflammation, necrosis), and the P-value cutoff ratio. Expression data that can be used with the K-nearest neighbor model and predictive genes to enable one skilled in the art to make predictions are given in Tables 28-30.

Referring now to Table 28, gene expression data for 6 hour timepoint are presented as mean ratio of treatment/control for all 6 hour predictive genes as presented in Table 18.

Referring now to Table 29, gene expression data for 24 hour timepoint are presented as mean ratio of treatment/control for all 24 hour predictive genes as presented in Table 5.

Referring now to Table 30, (1) gene expression data for 72 hour timepoint are presented as mean ratio of treatment/control for all 72 hour predictive genes as presented in Table 23. (2) Compound Dose indicates that compound and dose abbreviations are defined in Table 1. (3) Animal Number indicates the number of the individual animal in which the compound is tested. (4) Liver inflammation toxicity classification information as for compound-dose group at 72 h: yes-necr, indicates that necrosis was observed; yes-both, indicates that necrosis with inflammation was observed; no, indicates that no histopathology was observed. (5) Gene name is the Predictive gene (as in Table 23 and as included in Table 26).

The combined list of predictive genes or alternatively, Combo 5, 4, 3, 2, or 1 list or subsets thereof is used as input into the Predictive Model. As an external verification of the predictive abilities of the genes found to be predictive for liver inflammation, random lists of genes may be generated and also used as input into the Predictive Model. Example 2 describes the evaluation of the predictive performance of the liver inflammation predictive genes.

Predictive performance may also be assessed using data from different time points after exposure to the agent. In one embodiment, 24 hour expression data is used. In another embodiment, 6 hour expression data is used, as described in Examples 3 and 4. In another embodiment, 72 hour expression data is used, as described in Example 5 and 6. As illustrated in Table 9, the predictive accuracy using 24 hour expression data and the largest predictive gene list is about 86%.

Somewhat lower predictive accuracies were observed for the 6 h and 72 h data. All of the combo lists as well as Combo AII list had significantly higher accuracy than using random classifications.

Predictive performance may also be assessed using subsets of genes from the different Combo lists. As indicated in Example 2, most randomly selected subsets of the Combo gene lists yielded predictive performances of about 70% or greater and even individual genes had mean predictive accuracies that were often greater than about 70%. In one embodiment, using 10 genes from Combo AII yields about 84% accuracy. Using different Combo lists may require a greater number of genes to reach the same accuracy level.

The liver inflammation predictive genes disclosed herein and liver inflammation predictive genes identified by using methods disclosed herein are useful for predicting liver inflammation in response to exposure to one or more agents.

The discovery that relatively small sets of different genes have predictive value permits flexible applications. The choice of how many and which genes to use can be tailored to a variety of different purposes. Predictivity is observed for sets of a few genes. These small sets may be particularly advantageous in applications where measurement of only a few RNA species has considerable advantages in terms of sample processing logistics, speed and cost. These applications would include relatively high throughput screens for predictive capability. An example of this would be an early screen using small samples of primary cells or cultured cell lines that can be processed with automated robotic equipment for treatment and isolation of RNA followed by efficient technologies for measuring expression of a few RNA species such as branched chain technology or RT-PCR.

The use of larger numbers of predictive genes provides redundancy which may improve accuracy and precision. Applications using larger numbers of predictive genes may include, for example, tests of drug candidates at later stages of commercial development. In this regard, larger numbers of predictive genes may be desirable at later stages of preclinical development of a therapeutic candidate, where in vivo samples can be obtained and more comprehensive methods such as microarray measurement of gene expression are appropriate. The larger gene sets can also include different subsets of genes which may offer more insight into potential mechanisms of toxicity, providing the potential to predict long term toxic consequences such as chronic, irreversible toxicity or carcinogenicity.

Some genes within the liver inflammation predictive gene sets provided herein may also be suitable for prediction of toxicity in other organs or may be preferable for predicting toxicity for wider ranges of timepoints or treatment routes or regimens. As an example of the latter, some of the predictive genes are observed at three different timepoints after treatment. These genes may be useful for prediction in cases where the samples come from treatment protocols that have different measurement timepoints or routes of administration than those employed for the database used in the discovery of the predictive genes disclosed herein or where the toxicokinetics for a particular agent are known or suspected to be different from those in the database.

In one embodiment, the agent is an agent for which no expression profile has been assessed or stored in the database or library. An animal, e.g., rat, is dosed with such an agent and the gene expression profile(s) is the test set for the Predictive Model. The training set which is used in the Predictive Model in this case can be the entire database of sample array data because the test set data is not present in the database. The prediction can be made with accuracy without the use of histopathology scores as part of the input into the Predictive Model.

In another embodiment the agent is an agent present in the database but is used at a different dose level or with a different treatment protocol than used in the database. The training set which is used in the Predictive Model in this case can be the entire database of sample array data because the test set data is not present in the database. Again, the prediction can be made with accuracy without the use of histopathology scores as part of the input into the Predictive Model.

In another embodiment, the exposure time of the agent is other than 6, 24, or 72 hours, or repeat dosing protocols are used. In this case, the skilled artisan can use the predictive toxicity genes from surrounding time points to extrapolate the predicted toxicity without undue experimentation. For example, if the individual has been exposed to the agent for 12 hours, then predictive genes from 6 and 24 hours timepoints are used as guidelines for extrapolating toxicity predictions.

In another embodiment, the liver inflammation predictive genes and a predictive model can be used to determine the presence or absence of a no-observed toxicity effect level. An agent can be used at different treatment levels and expression profiles obtained for each treatment level. The predictive genes and predictive model can be used to determine which dose levels elicit a response that is predicted to be toxic and which dose levels are not toxic. In contrast to conventional endpoints for determining no-effect levels, the use of expression data, predictive genes and predictive models applies a number of quantitative endpoints and criteria instead of subjective endpoints and criteria. This permits more rigorous and precisely defined determination of no effect levels.

In another embodiment, the liver inflammation predictive genes can be used to detect toxic effects that may be manifested as long lasting or chronic consequences such as irreversible toxicity or carcinogenesis. The predictive genes and model can be applied to databases where classifications of training and test set samples are made with respect to actual or putative endpoints such as irreversible toxicity or carcinogenicity.

In another embodiment, the predictive genes can be used in a variety of alternative models to predict liver inflammation. Some of these models do not require the direct use of data in a database but use functions or coefficients derived from the database. In another embodiment, the predictive genes and models may be used to evaluate in vitro systems for their ability to reflect in vivo toxic events and to use such in vitro systems for predicting in vivo toxicity. Expression profiles for predictive genes can be created from candidate in vitro assays using treatments with agents of known in vivo toxicity and for which in vivo data on gene expression are available. The expression data and predictive models of this invention can be used to determine whether the in vitro assay system has predictive gene expression responses that accurately reflect the in vivo situation. Large sets of predictive genes as described in one embodiment of the present invention can be tested in such models for their suitability and performance with the candidate in vitro systems. This is a superior and novel tool for evaluating and optimizing in vitro systems for their ability to reflect and accurately predict in vivo responses.

In another embodiment, the predictive genes and models may be used with an in vitro system to accurately predict in vivo toxicity. In vitro systems that have been evaluated and optimized as described above are treated with test agents and expression profiles are measured for predictive genes. The expression profiles are used in conjunction with a predictive model to predict in vivo toxicity. In this embodiment, there can be considerable reduction in the use of laboratory animals. Additionally the application of this embodiment to in vitro human systems can provide a unique capability to accurately predict human toxic responses without human in vivo exposure or treatment.

In another embodiment, measurement of the expression levels of the proteins encoded by the predictive genes can be used in conjunction with predictive models to predict toxicity. Among the full set of liver inflammation predictive genes are various genes known to encode cell surface, secreted and/or shed proteins. This enables the development of methods for predicting toxicity using protein biomarkers. For example, as disclosed in Table 27, there are 39 genes in the master predictive set which are known to encode secreted proteins. The protein products are easier to access since they are secreted into body fluids and are thus more amenable to be quantified. Thus, in another aspect of the present invention, liver inflammation predictive assays which detect the expression of one or more of said predictive proteins may be developed. Such assays may have several advantages, such as:

Ability to use archived tissue specimens such as preserved or embedded tissues which are not suitable for measurement of RNA expression.

Ability to examine predictive protein expression in tissue slides using in situ labeling and microscopic observation. This is useful for detecting predictive toxicity signals occurring in very small sub-populations of cells.

Ability to detect protein markers in specimens that can be readily obtained with little or no invasiveness (e.g., blood, urine, sweat, saliva).

Reduction in animal use in laboratory studies such that no sacrifice of animals necessary to obtain tissue specimens when toxicity prediction can be made with specimens that can be obtained without animal sacrifice or surgery.

Application for human use where tissue specimens cannot be obtained or are only obtained with great difficulty.

In another embodiment, the identified predictive genes can be considered as potential therapeutic targets when the genes are involved in toxic damage or repair responses whose expression or functional modification may attenuate, ameliorate or eliminate disease, conditions or adverse symptoms of disease conditions.

In another embodiment the predictive genes can be organized into clusters of genes that exhibit similar patterns of expression by a variety of statistical procedures commonly used to identify such coordinate expression patterns. Common functional properties of these clustered genes can be used to provide insight into the functional relationship of the response of these genes to toxic effects. Common genetic properties of these genes (e.g., common regulatory sequences) may provide insight into functional aspects by revealing known or novel similarities in the coding region of the genes. The presence of common known or novel signal transduction systems that regulate expression of the genes can also provide functional insight. The presence of common known or novel regulatory sequences in the identified predictive genes can also be used to identify additional liver inflammation predictive genes.

In yet another embodiment, the liver inflammation predictive genes can be used to predict toxicity responses in other species, for example, human, non-human primate, mouse, hamster, guinea pig, hamster, rabbit, cattle, sheep, pig, chicken, and dog. Some members of the liver inflammation predictive genes may also be more suitable for prediction of toxicity in species other than the species used to derive the database (rat in the case of the examples provided). One method for identifying such genes involves examining DNA sequence databases to identify and characterize orthologous sequences to the predictive genes in the target species. One of skill in the art can examine the orthologous sequences for similarity in amino acid coding regions and motifs as well as for similarities in regulatory regions and motifs of the gene.

In another embodiment, liver inflammation predictive genes or gene sequences are used for screening other potential toxicity predictive genes or gene sequences in other species or even within the same species using methods known in the art. See, for example, Sambrook supra. Gene sequences which hybridize under stringent conditions to the liver inflammation predictive gene sequences disclosed herein may be selected as potential toxicity predictive genes. Additionally, genes which demonstrate significant homology with the liver inflammation predictive genes disclosed herein (preferably at least about 70%) may be selected as toxicity predictive gene candidates. It is understood that conservative substitutions of amino acids are possible for gene sequences which have some percentage homology with the liver inflammation predictive gene sequences of this invention. A conservative substitution in a protein is a substitution of one amino acid with an amino acid with similar size and charge. Groups of amino acids known normally to be equivalent are: (a) Ala, Ser, Thr, Pro, and Gly; (b) Asn, Asp, Glu, and Gln; (c) His, Arg, and Lys; (d) Met, Glu, Ile, and Val; and (e) Phe, Tyr, and Trp.

It is understood that the predictive liver inflammation genes can be used as guides to predicting toxicity for agents that have been administered via different routes (intraperitoneal, intravenous, oral, dermal, inhalation, mucosal, etc.) from the routes that were used to generate the database or to identify the liver inflammation predictive genes. Furthermore, the invention is not intended to be limiting to agents that have been administered at different dosages than the agents that were used to generate the database or to identify the predictive liver inflammation genes.

Data described in the examples were generated using the microarray technology disclosed in the Examples. However, the invention is not dependent on using this particular platform. Other similar gene expression analysis technologies may be incorporated in the practice of this invention. These can include, but are not limited to, other arrays containing the predictive genes, RT-PCR (e.g., TaqMan®), branched chain technology, RNAse protection or any other method which quantitatively detects the expression of RNA polynucleotides. Embodiments of the present invention can be practiced using these other technologies by generating a database of expression measurements for the predictive genes using samples such as those used in the database described in Example 1. This database can then be used in a model such as the K-nearest neighbor model or can be used to develop any of a number of other models.

The following Examples are provided to illustrate but not to limit the invention in any manner.

### EXAMPLES

### Example 1

### Database of Compounds and Liver Inflammation

Compounds and treatments list used to construct the liver database are given in Table 1. This table also provides the evaluation of the liver inflammation observed in samples collected 72 hours after treatment.

Sprague Dawley rats Crl:CD from Charles River, Raleigh, N.C. were divided into treated rats that receive a specific concentration of the compound (see Table 1) and the control rats that only received the vehicle in which the compound is mixed (e.g., saline).

At specified timepoints (6 h, 24 h and 72 h) after administration (intraperitoneal route) of the compound, a set number of rats (usually 3 control and 3 treated) were euthanized and tissues collected. Each rat was heavily sedated with an overdose of CO₂ by inhalation and a maximum amount of blood drawn. Exsanguination of the rat by this drawing of blood kills the rat. The method of collecting the tissues is very important and ensures preserving the quality of the mRNA in the tissues. The body of the rat was then opened up and prosectors rapidly removed the tissues (including liver) and immediately placed them into liquid nitrogen. All of the organs/tissues were completely frozen within 3 minutes of the death of the animal to ensure that mRNA did not degrade. The organs/tissues were then packaged into well-labeled plastic freezer quality bags and stored at −80 degrees until needed for isolation of the mRNA from a portion of the organ/tissue sample.

Isolating DNA/RNA from animal tissues or cells: Total RNA was isolated from liver tissue samples using the following materials: Qiagen RNeasy midi kits, 2-mercaptoethanol, liquid N₂, tissue homogenizer, dry ice samples were kept on ice when specified.

If a tissue needed to be broken, then the tissue sample was placed on a double layer of aluminum foil which was then placed within a weigh boat containing a small amount of liquid nitrogen. The aluminum foil was folded around the tissue and then struck by a small foil-wrapped hammer to administer mechanical stress forces.

About 0.15-0.20 g of liver tissue was weighed out and placed in a sterile container. To preserve integrity of the RNA, all tissues were kept on dry ice when other samples were being weighed. A RLT (Qiagen®) buffer was added to the sample to aid in the homogenization process. The tissue was homogenized using commercially available homogenizer (IKA Ultra Turrax T25 homogenizer) with the 7 mm microfine sawtooth shaft and generator (195 mm long with a processing range of 0.25 ml to 20 ml, item # 372718). After homogenization, samples were stored on ice until all samples were homogenized. The homogenized tissue sample was spun to remove nuclei thus reducing DNA contamination. The supernatant of the lysate was then transferred to a clean container containing an equal volume of 70% EtOH in DEPC treated H₂O and mixed. RNA was isolated by putting the supernatant through an RNeasy spin column, washed, and subsequently eluted. Small quantities of remaining DNA were removed by use of DNase enzyme during the RNA isolation procedure following the instructions provided by Qiagen and alternatively by lithium chloride (LiCl) precipitation following the RNA isolation. The isolated RNA pellet was stored in Rnase-free water or in an RNA storage buffer (10 mM sodium citrate), Ambion Cat #7000. The RNA amount was then quantitated using a spectrophotometer.

Rat 700 CT chip: Gene expression data was generated from a microarray chip that has a set of toxicologically relevant rat genes which are used to predict toxicological responses. The rat 700 CT gene array is disclosed in pending U.S. applications 60/264,933; 60/308,161; and pending application filed on Jan. 29, 2002 (Ser. No. 10/060,893).

Microarray RT reaction: Fluorescence-labeled first strand Cdna probe was made from the total RNA or Mrna isolated from livers of control and treated rats. This probe was hybridized to microarray slides spotted with DNA specific for toxicologically relevant genes. The materials needed are: total or messenger RNA, primer, Superscript II buffer, dithiothreitol (DTT), nucleotide mix, Cy3 or Cy5 dye, Superscript 11 (RT), ammonium acetate, 70% EtOH, PCR machine, and ice.

The volume of each sample that would contain 20 μg of total RNA (or 2 μg of Mrna) was calculated. The amount of DEPC water needed to bring the total volume of each RNA sample to 14 μl was also calculated. If RNA was too dilute, the samples were concentrated to a volume of less than 14 μl in a speedvac without heat. The speedvac must be capable of generating a vacuum of 0 Milli-Torr so that samples can freeze dry under these conditions. Sufficient volume of DEPC water was added to bring the total volume of each RNA sample to 14 μl. Each PCR tube was labeled with the name of the sample or control reaction. The appropriate volume of DEPC water and 8 μl of anchored oligo Dt mix (stored at −20° C.) was added to each tube.

Then the appropriate volume of each RNA sample was added to the labeled PCR tube. The samples were mixed by pipeting. The tubes were kept on ice until all samples are ready for the next step. It is preferable for the tubes to kept on ice until the next step is ready to proceed. The samples were incubated in a PCR machine for 10 minutes at 70° C. followed by 4° C. incubation period until the sample tubes were ready to be retrieved. The sample tubes were left at 4° C. for at least 2 minutes.

The Cy dyes are light sensitive, so any solutions or samples containing Cy-dyes should be kept out of light as much as possible (e.g., cover with foil) after this point in the process. Sufficient amounts of Cy3 and Cy5 reverse transcription mix were prepared for one to two more reactions than would actually be run by scaling up the following: For labeling with Cy3:

8 ul 5×First Strand Buffer for Superscript II, ul 0.1 M DTT, 2 ul Nucleotide Mix, 2 ul of 1:8 dilution of Cy3 (e.g., 0.125 Mm cy3Dctp), and 2 ul Superscript II

For labeling with Cy5.

8 ul 5× First Strand Buffer for Superscript II, 4 ul 0.1 M DTT, 2 ul Nucleotide Mix, 2 ul of 1:10 dilution of Cy5 (e.g., 0.1 Mm Cy5Dctp), and 2 ul Superscript II

About 18 μl of the pink Cy3 mix was added to each treated sample and 18 μl of the blue Cy5 mix was added to each control sample. Each sample was mixed by pipeting. The samples were placed in a DNA engine (PTC-200 Petier Thermal Cycler, MJ Research) for 2 hours at 45° C. followed by 4° C. until the sample tubes were ready to be retrieved.

In addition to the desired cDNA product, the completed RT reaction contained impurities that must be removed. These impurities included excess primers, nucleotides, and dyes. The primary method of removing the impurities was by following the instructions in the OIAquick PCR purification kit (Qiagen cat#120016).

Alternatively, the completed RT reactions were cleaned of impurities by ethanol precipitation and resin bead binding. The samples from DNA engine were transferred to Eppendorf tubes containing 600 μl of ethanol precipitation mixture and placed in —80° C. freezer for at least 20-30 minutes. These samples were centrifuged for 15 minutes at 20800× g (14000 rpm in Eppendorf model 5417C) and carefully the supernatant was decanted. A visible pellet was seen (pink/red for Cy3, blue for Cy5). Ice cold 70% EtOH (about 1 ml per tube) was used to wash the tubes and the tubes were subsequently inverted to clean tube and pellet. The tubes were centrifuged for 10 minutes at 20800×g (14000 rpm in Eppendorf model 5417C), then the supernatant was carefully decanted. The tubes were air dried for about 5 to 10 minutes, protected from light. When the pellets were dried, they were resuspended in 80 ul nanopure water. The cDNA/mRNA hybrid was denatured by heating for 5 minutes at 95° C. in a heat block and flash spun. Then the lid of a “Millipore MAHV N45” 96 well plate was labeled with the appropriate sample numbers. A blue gasket and waste plate (v-bottom 96 well) was attached. About 160 μl of Wizard DNA Binding Resin (Promega cat#A1151) was added to each well of the filter plate that was used. Probes were added to the appropriate wells (80 μl cDNA samples) containing the Binding Resin. The reaction is mixed by pipeting up and down ˜10 times. The plates were centrifuged at 2500 rpm for 5 minutes (Beckman GS-6 or equivalent) and then the filtrate was decanted. About 200 μl of 80% isopropanol was added, the plates were spun for 5 minutes at 2500 rpm, and the filtrate was discarded. Then the 80% isopropanol wash and spin step was repeated. The filter plate was placed on a clean collection plate (v-bottom 96 well) and 80 μl of Nanopure water, pH 8.0-8.5 was added. The pH was adjusted with NaOH. The filter plate was secured to the collection plate and after 5 minutes was centrifuged for 7 minutes at 2500 rpm.

Purification of Cy—Dye Labeled cDNA: To purify fluorescence-labeled first strand cDNA probes, the following materials were used: Millipore MAHV N45 96 well plate, v-bottom 96 well plate (Costar), Wizard DNA binding Resin, wide orifice pipette tips for 200 to 300 μl volumes, isopropanol, nanopure water. It is highly preferable to keep the plates aligned at all times during centrifugation. Misaligned plates lead to sample cross contamination and/or sample loss. It is also important that plate carriers are seated properly in the centrifuge rotor.

The lid of a “Millipore MAHV N45” 96 well plate was labeled with the appropriate sample numbers. A blue gasket and waste plate (v-bottom 96 well) was attached. Wizard DNA Binding Resin (Promega cat#A1151) was shaken immediately prior to use for thorough resuspension. About 160 μl of Wizard DNA Binding Resin was added to each well of the filter plate that was used. If this was done with a multi-channel pipette, wide orifice pipette tips would have been used to prevent clogging. It is highly preferable not to touch or puncture the membrane of the filter plate with a pipette tip. Probes were added to the appropriate wells (80 μl cDNA samples) containing the Binding Resin. The reaction is mixed by pipeting up and down ˜10 times. It is preferable to use regular, unfiltered pipette tips for this step. The plates were centrifuged at 2500 rpm for 5 minutes (Beckman GS-6 or equivalent) and then the filtrate was decanted. About 200 μl of 80% isopropanol was added, the plates were spun for 5 minutes at 2500 rpm, and the filtrate was discarded. Then the 80% isopropanol wash and spin step was repeated. The filter plate was placed on a clean collection plate (v-bottom 96 well) and 80 μl of Nanopure water, pH 8.0-8.5 was added. The pH was adjusted with NaOH. The filter plate was secured to the collection plate with tape to ensure that the plate did not slide during the final spin. The plate sat for 5 minutes and was centrifuged for 7 minutes at 2500 rpm. Replicates of samples should be pooled.

Dry-down Process: Concentration of the cDNA probes is preferable so that they can be resuspended in hybridization buffer at the appropriate volume. The volume of the control cDNA (Cy-5) was measured and divided by the number of samples to determine the appropriate amount to add to each test cDNA (Cy-3). Eppendorf tubes were labeled for each test sample and the appropriate amount of control cDNA was allocated into each tube. The test samples (Cy-3) were added to the appropriate tubes. These tubes were placed in a speed-vac to dry down, with foil covering any windows on the speed vac. At this point, heat (45° C.) may be used to expedite the drying process. Samples may be saved in dried form at −20° C. for up to 14 days.

Microarray Hybridization: To hybridize labeled CDNA probes to single stranded, covalently bound DNA target genes on glass slide microarrays, the following material were used: formamide, SSC, SDS, 2 μm syringe filter, salmon sperm DNA (Sigma, cat # D-7656), human Cot-1 DNA (Life Technologies, cat # 15279-011), poly A (40 mer: Life Technologies, custom synthesized), yeast tRNA (Life Technologies, cat # 15401-04), hybridization chambers, incubator, coverslips, parafilm, heat blocks. It is preferable that the array is completely covered to ensure proper hybridization.

About 30 μl of hybridization buffer was prepared per cDNA sample (control rat cDNA plus treated rat cDNA). Slightly more than is what is needed should be made since about 100 μl of the total volume made for all hybridizations can be lost during filtration.

| Hybridization Buffer: | for 100 μl: |
|---|---|
| 50% Formamide | 50 μl formamide |
| 5 × SSC | 25 μl 20 × SSC |
| 0.1% SDS | 25 μl 0.4% SDS |

The solution was filtered through 0.2 μm syringe filter, then the volume was measured. About 1 μl of salmon sperm DNA (10 mg/ml) was added per 100 μl of buffer.

Alternatively, the hybridization buffer was made up as:

| Hybridization Buffer: | for 101 μl: |
|---|---|
| 50% Formamide | 50 μl formamide |
| 10 × SSC | 50 μl 20 × SSC |
| 0.2% SDS | 1 μl 20% SDS |

The solution was filtered through 0.2 μm syringe filter, then the volume was measured. One microliter of salmon sperm DNA (9.7 mg/ml), 0.5 μl Human Cot-1 DNA (5 μg/μl), 0.5 μl poly A (5 μg/μl), 0.25 μl Yeast tRNA (10 μg/μl) was added per 100 μl of buffer. The hybridization buffers were compared in validation studies and there was no change in differential gene expression data between the two buffers.

Materials used for hybridization were: 2 Eppendorf tube racks, hybridization chambers (2 arrays per chamber), slides, coverslips, and parafilm. About 30 μl of nanopure water was added to each hybridization chamber. Slides and coverslips were cleaned using N₂ stream. About 30 μl of hybridization buffer was added to dried probe and vortexed gently for 5 seconds. The probe remained in the dark for 10-15 minutes at room temperature and then was gently vortexed for several seconds and then was flash spun in the microfuge. The probes were boiled or placed in a 95° C. heat block for 5 minutes and centrifuged for 3 min at 20800×g (14000 rpm, Eppendorf model 5417C). Probes were placed in 70° C. heat block. Each probe remained in this heat block until it was ready for hybridization.

About 25 μl was pipeted onto a coverslip. It is highly preferable to avoid the material at the bottom of the tube and to avoid generating air bubbles. This may mean leaving about 1 μl remaining in the pipette tip. The slide was gently lowered, face side down, onto the sample so that the coverslip covered that portion of the slide containing the array. Slides were placed in a hybridization chamber (2 per chamber). The lid of the chamber was wrapped with parafilm and the slides were placed in a 42° C. humidity chamber in a 42° C. incubator. It is preferable to not let probes or slides sit at room temperature for long periods. The slides were incubated for 18-24 hours.

Post-Hybridization Washing: To obtain only single stranded cDNA probes tightly bound to the sense strand of target cDNA on the array, all non-specifically bound cDNA probe should be removed from the array. Removal of all non-specifically bound cDNA probe was accomplished by washing the array and using the following materials: slide holder, glass washing dish, SSC, SDS, and nanopure water. Six glass buffer chambers and glass slide holders were set up with 2×SSC buffer heated to 30-34° C. and used to fill up glass dish to ¾th of volume or enough to submerge the microarrays. The slides were placed in 2×SSC buffer for 2 to 4 minutes while the cover slips fall off. The slides were then moved to 2×SSC, 0.1% SDS and soaked for 5 minutes. The slides were transferred into 0.1×SSC and 0.1% SDS for 5 minutes. Then the slides are transferred to 0.1×SSC for 5 minutes. The slides, still in the slide carrier, were transferred into nanopure water (18 megaohms) for 1 second. To dry the slides, the stainless steel slide carriers were placed on micro-carrier plates and spun in a centrifuge (Beckman GS-6 or equivalent) for 5 minutes at 1000 rpm.

The washed and dried hybridized slides were scanned on Axon Instruments Inc. GenePix 4000A MicroArray Scanner and the fluorescent readings from this scanner converted into quantitation files (.gpr) on a computer using GenePix software.

Array Data, Normalization and Transformation: GeneSpring™ software (Version 4.1, Silicon Genetics) was used for statistical analyses including identification of genes expressions correlating with histopathology scores, K-means and tree cluster analysis, and predictive modeling using the k nearest neighbor (Predict Parameter Values tool).

Microarray data were loaded into GeneSpring™ software for analysis as GenePix files as above. Specific data loaded into GeneSpring™ software included gene name, GenBank ID control channel mean fluorescence and signal channel mean fluorescence. Expression ratio data (ratio of signal to control fluorescence) were normalized using the 50^{th} percentile of the distribution of all genes and control channel. Ratio data were excluded from analysis if the control channel value was <0. For analysis of correlations and predictive values gene expression ratios were transformed as the log of the ratio.

Correlation with Histopathology Scores: Histopathology scores for each animal (assigned on a compound-dose basis as indicated in Table 1) were entered with gene expression data by using the GeneSpring™ ‘Drawn Gene’ function. Correlations between inflammation histopathology scores and gene expression were conducted with the distance measures listed below:

| | |
|---|---|
| standard | positive and negative correlation |
| smooth | positive and negative correlation |
| change | positive correlation |
| upregulated | positive correlation |
| Pearson | positive and negative correlation |
| Spearman | positive and negative correlation |
| distance | positive correlation |

These correlation or similarity measures are standard statistical correlation measures that are described in the GeneSpring Advanced Analysis Techniques Manual (Release Date Mar. 13, 2001, Silicon Genetics). Where both positive and negative correlations were obtained combined positive and negative correlating gene lists were also created.

The Predict Parameter Values tool in GeneSpring™ software was used for liver inflammation class prediction. The following is a summary of the procedure used in the GeneSpring predictive software. This is described in GeneSpring Advanced Analysis Techniques Manual (Release Date Mar. 13, 2001, Silicon Genetics) with additional information supplied by Silicon Genetics and a statistical expert. The prediction tool relies on standard statistical procedures that can be implemented in a variety of statistical software packages.

Gene Selection: The first step is variable selection of genes to be used for prediction. This entails taking a single gene and a single class (e.g., liver inflammation) and creating a contingency table. In the table below, columns 1 through N of the table each represent one possible cutoff point based on the gene expression level (ratio of signal/control) for that class. The number of possible cutoffs is less than or equal to the total number of samples for the class (e.g., A). It is possibly less than the total number, since there may be ties in gene expression level. Hence, N, M, and X may or may not be distinct. In the example, an n-class problem is illustrated, where x and y entries are the class counts at that gene expression cutoff level, for that specific gene and class, either above (“a”) or below (“b”) the cutoff. “Class1” is the set of all samples (above or below) the cutoff for Class1, and “!Classl” are all those not in Class1 (above or below) the cutoff, and similarly for the other classes. The class totals in the training set are the total class marginals used to compute Fisher's exact test.

For a specific gene, and for each class, the best p-value as calculated by Fisher's Exact Test for independence between one of the pair of columns (e.g., 1a and 1b) and the actual class totals (e.g., A) is used to score the gene (-In(p)=the score) for that class. Thus, there are N (or, M, 0 etc.) contingency tables, where the best score of the N tables is used for that class and gene. If there is a wide disparity between the above and below counts in either the a or b column (this is a two-sided Fisher's Exact Test), the smaller the p-value and the higher the score.

The genes per class are rank ordered by the most discriminating (highest) score. The predictivity list is composed of the most discriminating genes per class. Namely, genes are combined that best discriminate class 1 with those that best discriminate class 2 and so on. The genes are selected in rotation of the highest score per class. Duplicate genes are ignored in the rotation and not added to the list, the gene with the next highest score is taken.

The training samples now have only the gene list garnered from the above procedure. As an example, where once the training samples may have had an initial list of 200 genes per sample, they now have only a subset composed of the gene list, say, 60 (the number of predictivity genes specified) that are selected from the initial list by the gene selections procedure. Thus, each sample is a vector of 60 normalized expression ratios. Since the selection of genes is done in rotation, for 2 classes, the list contains 30 genes for class one, and 30 genes for class two. For 3 classes the list contains 20 genes for class one, 20 for class two, and 20 for class three, etc. The matrix below illustrates the basic features of this gene selection process.

| | | | | | | |
|---|---|---|---|---|---|---|
| Gene 1 | 1a | 1b | . . . | Na | Na | |
| | | | | | | Actual Class |
| | Expression | Expression | | Expression | Expression | Totals |
| Class | above | below | . . . | above | below | (Marginals) |
| Class1 | x1.1a | x1.1b | . . . | x1.Na | x1.Nb | A |
| !Class1 | y1.1a | y1.1b | . . . | y1.Na | y1.Nb | B |
| Gene 1 | 1 | 2 | . . . | M | | |
| Class2 | x1.2a | x1.2b | . . . | x1.Ma | | C |
| !Class2 | y1.2a | y1.2b | . . . | y1.Ma | | D |
| . | . | . | . | . | | . |
| . | . | . | . | . | | . |
| . | . | . | . | . | | . |
| Gene 1 | 1 | 2 | . . . | Qa | Qb | |
| Classn | x1.na | x1.nb | . . . | x1.Qa | x1.Qb | X |
| !Classn | y1.na | y1.nb | . . . | y1.Qa | y1.Qb | Y |

After the genes to be used in the training set have been selected, the test set is classified based on the k-nearest neighbor (knn) voting procedure. Using just those genes in the gene list, for each sample in the test set of samples, the k nearest neighbors in the training set are found with the Euclidean distance. The class in which each of the k nearest neighbors is determined, and the test set sample is assigned to the class with the largest representation in the k nearest neighbors after adjusting for the proportion of classes in the training set.

For example, in a two-class problem, let there be 30 samples of class 1 and 60 samples of class 2 in the training set. With k=9 say it can be determined that 7 of the nearest neighbors to a sample from the testing set are in class 1. The sample can then be classified as being a member of class 1. If another sample from the test set has a total of 4 nearest neighbors in class 1, after adjusting for the proportion, this sample would be assigned to class 1 rather than class 2, even though the majority vote suggests assignation to class 2.

The decision threshold is a mechanism to help clearly define the class into which the sample will fall, and can be set to reject classification if the voting is very close or tied. (Thus, k can be even for two-class problems without worrying about the tie problem.) A p-value is calculated for the proportion of neighbors in each class against the proportions found in the training set, again using Fisher's exact test, but now a one-sided test.

For example, let k=11, if the proportion of neighbors of class 1 in the test set is 6/11, and the proportion of class 1 in a 100 sample training set is 0.4, the p-value calculated is 0.29 (half the two-sided test). If the proportion in the training set is 0.1, the p-value is 0.004. The smaller the p-value the greater the likelihood that the sample from the testing set belongs to that class.

A p-value ratio (P-value) is set as a way of setting the level of confidence in individual sample predictions based on the ratio of p-values for the best class (lowest p-value) versus the second best class (second lowest p-value). For example, if the P-value is set at 0.5 and the ratio of p-values for a particular sample is 0.6, then the predictive model will not make a call for that sample.

Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in Table 2.

Liver inflammation classifications were entered for training and test set as a parameter column. Toxicity, as defined by observation of liver necrosis or necrosis with inflammation at 72 hours after treatment, was entered as “negative”, “positive-necrosis”, or “positive-necrosis with inflammation” for each animal in a compound-dose group. Additionally, a parameter column for random histopathology classification was designated. This was done by randomly assigning the same number of “negative”, “positive-necrosis”, or “positive-necrosis with inflammation” calls to the individual animals.

The “Predict Parameter Value” tool of GeneSpring was used with each of the training and test sets to generate predictions of histopathology classifications of the test sets. The number of k nearest neighbors was optimized to give the highest predictive accuracy. This was done by first running predictions at different nearest neighbors for three of the training and test sets, and then evaluating the overall predictive performance for each number of nearest neighbors. A P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct classifications/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Expression array data were first examined for the existence of genes whose expression correlated with histopathology scores. Table 1 presents a list of the compounds and dose levels along with the liver histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 3 and 4.

The correlating gene lists as well as the entire array gene list were provided as input lists to the GeneSpring Predict Parameter value tool (described in Materials and Methods) that employs a k nearest neighbor (knn) predictive model. These lists as well as the entire array gene list were used for each of the five training and test sets defined in Materials and Methods to generate predictions of histopathology classifications of the test sets. Input genes for the Predict Parameter Value feature included all 700 genes in the GenePix file (the rat CT Array) which were disclosed in a currently pending application (Ser. No. 10/060,893) filed on Jan. 29, 2002, as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes.

After this was done for all 5 training and test sets, all gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in all 5 training and test sets were designated as Combo (combination) 5. Genes that were predictive in only 4 of 5 training and test sets were designated as Combo 4, etc. A list of predictive genes organized by their occurrence in the separate training and test sets is presented in Table 5. The combination category is the number of training/test set gene lists occurrences.

### Example 2

The database used was as described in Example 1.

Array data, normalization procedures and transformations used in these analyses are as described in Example 1. Table 29 presents 24 hour gene expression data for the predictive genes. These data can be used with a k nearest neighbor prediction model (as available in GeneSpring or other statistical software packages) to make predictions as described in this example.

The Predict Parameter Values tool in GeneSpring™ software_was used for liver inflammation class prediction. A description of this tool and the statistical procedures used is provided in Example 1.

The training and test data sets used are those described in Table 2 of Example 1.

Liver inflammation classifications used are described in Table 1 of Example 1. In this analysis randomized classifications (same number of “negative”, “positive-necrosis”, or “positive-necrosis with inflammation” classifications distributed randomly among the samples) were also used.

Prediction Output and Initial Data Processing: For each predicting gene list used for evaluation a table of data generated by the Predict Parameter Values tool in GeneSpring™ software was saved which provided for each sample in the test set the actual call (“negative”, “positive-necrosis with inflammation”, or “positive-necrosis”), the predicted call (“negative”, “positive-necrosis with inflammation”, or “positive-necrosis”) and the P-value cutoff ratio. This set of data was used to calculate predictive performance measures provided below.

Measures of prediction used for these analyses are generally accepted prediction measures for information about actual and predicted classifications done by a classification system (Modern Applied Statistics with S-Plus, W. N. and B. D. Ripley, Springer, 1994, 3rd edition.; Proc. 14th International Conference on Machine Learning, Miroslav Kubat, Stan Matwin, 1997). Results from predictions of a three class case can be described as a three-class matrix:

Predicted

| | | |
|---|---|---|
| Class I | Class II | Class III |

Actual

| | | | |
|---|---|---|---|
| Class I | a | b | c |
| Class II | d | e | f |
| Class III | g | h | i |

Class I is defined as “negative-no histopathology.”

Class II is defined as “positive-necrosis with inflammation”

Class III is defined as “positive-necrosis”.

Standard terms used for prediction for the three class case are:

Overall Accuracy is the proportion of total number of predictions that are correct=(a+e+i)/(a+b+c+d+e+f+g+h+i)

False Positive (Inflammation) rate (FPI) is the proportion of cases that are negative for inflammation (Class I or Class II) incorrectly classified as being positive for inflammation (Class 11)=(b+h)/(a+b+c+g+h+i)

False Negative (Inflammation) rate (FN_{I}) is the proportion of cases correctly classified as being positive for inflammation (Class II) that are incorrectly classified as negative for inflammation (Class I or Class II)=(d+f)/(d+e+f)

Geometric-mean is the performance measure that takes into account proportion of positive and negative cases (Kubat et al., ibid).

Geometric-mean (Inflammation) (GMM_{I}), which takes into account the proportion of positive and negative cases for inflammation, equals the square root of TP_{I}*TN_{I} where TP_{I}=True Positive (Inflammation) rate (e/(d+e+f)) and TN_{I}=True Negative (Inflammation) rate ((a+i)/(a+b+c+g+h+i)).

Geometric-mean (Necrosis) (GMMN), which takes into account the proportion of positive and negative cases for necrosis, equals the square root of TPN*TNN where TPN=True Positive (Necrosis) rate ((h+i)/(g+h+i)) and TNN=True Negative (Necrosis) rate ((a)/(a+b+c)).

In these analyses cases where no prediction was made because the p-value ratio exceeded the cutoff-value (generally 0.5) the non-call was considered to be incorrect. Non-calls of Class I samples are assumed to be Class II. Non-calls of Class II or Class III samples are assumed to be Class I.

Random Selected Gene Sets: Subsets of randomly selected genes were prepared from the predictive gene sets to test whether such subsets would have predictive value., Assignments of genes to these subsets are presented in Tables 6-7. Genes were also randomly selected from the list of all genes excluding the 183 twenty-four hour predictive genes (also known as non-predictive genes) by assigning a random number to each gene, sorting by the random number and selecting the appropriate number of sorted genes. Assignments of genes to these subsets are presented in Table 8. The “*” identifies that the genes randomly selected from the Combo AII list of predictive genes (183 genes) assigning a random number to each gene, sorting by the random number and selecting the appropriate number of sorted genes. Results: Prediction results for 24 hour expression data using genes identified as predictive are presented in Table 9. Referring now to Table 9, “*” denotes that values are given as means and range of values (in parentheses) for five training/test sets using 24 hour array data and gene lists as presented in Table 5. Unit of prediction was the animal and the predictive classification was for liver inflammation or necrosis observed at 72 hours after treatment.

“**” denotes that standard prediction measures were used as defined in Materials and Methods above. These include:

Overall Accuracy=Proportion of total number of predictions that are correct; FP_{I}=False Positive (Inflammation) rate, the proportion of negative cases for inflammation that are incorrectly classified as positive for inflammation; FN=False Negative (Inflammation) rate, the proportion of positive cases for inflammation that are incorrectly classified as negative; GMM=Geometric Mean (Inflammation), performance measure that takes into account the proportion of positive and negative cases for inflammation; GMMN=Geometric Mean (Necrosis), performance measure that takes into account the proportion of positive and negative cases for necrosis. Non-calls are counted as incorrect predictions as defined in Materials and Methods.

These data indicate a high accuracy in predicting liver inflammation. Mean accuracies were 0.85 (85% accuracy) or better for the entire predictive gene list (Combo AII) and the top two Combo gene lists (Combo 5 and Combo 3), and were close to 0.80 (80% accuracy) for the remaining Combo gene lists (Combo 2 and Combo 1). Because these predictions were conducted with multiple training/test set combinations it is possible to obtain an indication of the variability in prediction rates and robustness of the prediction capabilities of these gene sets. For the Combo AII and other Combo lists the minimum predictive accuracy value for any one training and test set was greater than 0.70 (70%), with most lists giving 0.75 (75%) or better minimum accuracy. False positive and false negative prediction rates for inflammation (FP_{I} and FN_{I}, respectively) were generally low with means generally 0.17 (17%) or less for the Combo AII, 5, and 3 gene sets.

The Geometric Mean (Inflammation) (GMM_{I}) was used as an indication of predictive performance that includes consideration of the proportion of positive and negative cases for inflammation. All gene sets gave GMM_{I} measures>0.75 (75%), and the Combo AII, Combo 5, and Combo 3 gene sets had GMM_{I} measures>0.85. The Geometric Mean (Necrosis) (GMMN) was used as an indication of predictive performance that includes consideration of the proportion of positive and negative cases for necrosis. All gene sets gave GMMN measures>0.80 (80%). Together, both GMM measures indicate that the 24 hour gene sets can predict samples with necrosis or samples with necrosis with inflammation.

As described above, in those cases where no prediction was made because the p-value ratio exceeded the cutoff-value (generally 0.5) the non-call was considered to be incorrect.

Prediction results for 24 hour expression data using genes identified as predictive and the predicting unit of compound-dose are presented in Table 10. Referring now to Table 10, the “**” denotes that overall accuracy is defined as the proportion of the total number of predictions that are correct. Non-Calls are counted as incorrect predictions as defined in Materials and Methods. This prediction unit is probably the most relevant for toxicology prediction. The performance of the genes in predicting compound-dose toxicity is even better than predictions on an individual animal basis. These data indicate a high accuracy in predicting liver inflammation. Mean accuracy exceeded 0.86 (86% accuracy) for the entire predictive gene list (Combo AII) as well as Combo 5 and Combo 3, and was greater than 0.80 (80% accuracy) for Combo 2 and Combo 1. Variability in accuracy was low for most of the gene lists with >0.7 (70%) minimum accuracy for any single training and test set observed for the Combo AII and Combo 5, 3, 2 and 1 gene lists.

One noteworthy feature of the predictive capability is the ability to distinguish between effects of a compound at different dose levels. Five compounds (ANIT, APAP, CCL4, LPS, and TET) produced liver necrosis or necrosis with inflammation at the high dose but not at the low dose. The predictive gene sets were usually accurate in predicting toxicity at the high dose and predicting no toxicity at the low dose.

Prediction results for 24 hour expression data using genes identified as predictive and the predicting unit is compound are presented in Table 11. Referring to Table 11, denotes Overall Accuracy to be defined as the proportion of the total number of predictions that are correct. Non-Calls are counted as incorrect predictions as defined in Materials and Methods. Predictive performances on a compound basis were also good, with accuracies generally being at or above 0.8 (80%).

Table 12 and 13 show the level of predictive accuracy of individual genes of Combos 3 and 2, respectively, for 24 hour liver data. The tables show that overall, individual genes of the Combo groups did not perform as well as the combination as a whole, as the average predictive accuracy of individual genes versus the entire combo set was 64.6% vs. 84.9% for Combo 3, and 64.9% vs. 79.3% for Combo 2. The table also shows that while many of the individual genes of the Combo groups were predictive (e.g., accuracies as high as 77.5% for individual genes of Combo 3 and 85.9% for Combo 2), the predictive accuracy of individual genes rarely exceeded the predictive accuracy of the whole combination.

In order to assess the performance of subsets of genes, predictive performance was evaluated for subsets of genes randomly selected from the total combined predictive list (Combo AII) and the top Combo sets (as defined in Materials and Methods). Prediction results for 24 hour expression data using randomly selected subsets of genes are presented in Table 14. Referring to Table 14, “*” denotes the combo gene lists as in Table 5. For combo lists all genes were used or randomly selected subsets of genes in Table 6 and Table 7. Referring now to Table 6, the genes were randomly selected from the Combo AII list of predictive genes (183 genes) assigning a random number to each gene, sorting by the random number and selecting the appropriate number of sorted genes. Referring now to Table 7, the genes were randomly selected from the combined Combo 5 3 2 list of predictive genes (52 genes) assigning a random number to each gene, sorting by the random number and selecting the appropriate number of sorted genes. Referring now to Table 14, AII-Pred used genes randomly selected from genes that were present on the array but not in the predictive list. “** Overall Accuracy” is defined as the proportion of the total number of predictions that are correct. Non-calls are counted as incorrect predictions as defined in Materials and Methods. Accuracy was calculated for correct classifications of “negative,” “positive-necrosis with inflammation,” or “positive-necrosis,” assigned to the samples and for randomized classifications in the same proportions as the correct classifications. Values presented are the mean accuracy values for 5 training/test sets with minimum and maximum accuracy values. These data clearly indicate that smaller subsets of the Combo gene lists have predictive power. Table 14 also compares prediction accuracy for correct classification of liver inflammation and for the same proportion of positive and negative toxicity calls randomly assigned to the samples (random classification). For each gene set or subset predictions were made using the same five training/test sets as for the other prediction analyses. Additionally, sets of genes were randomly chosen from the array which were not identified on the list of 183 predictive genes at 24 hour (Example 1, Table 5).

It is clear from these data that the predictions with accurate classification are much better than predictions with randomized classification. This means that the predictive results are not simply due to chance and large data sets but are due to significant, meaningful predictive association between the gene expression of the predictive genes and the liver inflammation. The accuracy numbers for the gene sets selected from a list of all genes on the array minus the predictive genes are much lower than the Combo predictive lists and the random subsets of these predictive lists. This also verifies the predictive power of the identified predictive genes. The fact that the predictive numbers from these subsets are somewhat higher for accurate than random classification is likely due to some residual predictivity in these genes that is not very substantial.

### Example 3

Compounds and treatments list used to construct the liver database are given in Table 1 of Example 1. This table also provides the evaluation of liver toxicity as observed as necrosis or necrosis with inflammation in samples collected 72 hours after treatment. The database is described in detail in Example 1. This Example analyzes expression data from samples collected 6 hours after treatment.

Array data, normalization and transformation procedures used were as described in Example 1.

Procedures and methods for obtaining gene lists correlating with histopathology scores were as described in Example 1.

The Predict Parameter Values tool in GeneSpring™ software used for liver inflammation class prediction is described in detail in Material and Methods of Example 1.

Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in the following Table 15. Referring to Table 15, Low+defines low dose. High* defines high dose. Compounds* abbreviates for Compound, Dose, Abbreviation, etc, are defined in Table 1. **Negative are compounds that did not elicit histopathology (score=1). **Positive are compounds that did elicit histopathology (score of 2 or greater).

Liver inflammation classifications were entered for training and test sets as a parameter column. Toxicity, as defined by observation of liver necrosis or necrosis with inflammation at 72 hours after treatment, was entered as “negative”, “positive-necrosis”, or “positive-necrosis with inflammation” for each animal in a compound-dose group. Additionally, a parameter column for random histopathology classification was designated. This was done by randomly assigning the same number of “negative”, “positive-necrosis”, or “positive-necrosis with inflammation” calls to the individual animals.

The “Predict Parameter Value” tool of GeneSpring was used with each of the training and test sets to generate predictions of histopathology classifications of the test sets. The number of k nearest neighbors was optimized to give the highest predictive accuracy. This was done by first running predictions at different nearest neighbors for three of the training and test sets, and then evaluating the overall predictive performance for each number of nearest neighbors. A P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct classifications/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Results: Expression array data were first examined for the existence of genes whose expression correlated with histopathology scores. Table 1 in Materials and Methods of Example 1 presents a list of the compounds and dose levels along with the liver histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 16-17.

The correlating gene lists as well as the entire array gene list were provided as input lists to the GeneSpring Predict Parameter value tool (described in Materials and Methods) that employs a k nearest neighbor (knn) predictive model. These lists as well as the entire array gene list were used for each of the five training and test sets defined in Materials and Methods to generate predictions of histopathology classifications of the test sets. Input genes for the Predict Parameter Value feature included all 700 genes in the GenePix file (the Rat CT Array) as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes.

After this was done for all 5 training and test sets, all gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in all 5 training and test sets were designated as Combo (combination) 5. Genes that were predictive in only 4 of 5 training and test sets were designated as Combo 4, etc.

A list of predictive genes organized by their occurrence in the separate training and test sets is presented in Table 18. Referring now to Table 18, the Combination (No. of Occurrences) category, refers to the number of training/test set gene list occurrences.

### Example 4

Materials and Methods: The database used was as described in Example 1. This Example analyzes expression data from samples collected 6 hours after treatment

Array Data, Normalization and Transformation: Array data, normalization procedures and transformations used in these analyses are as described in Example 1. Table 28 lists 6 hour gene expression data for the predictive genes. These data can be used with a k nearest neighbor prediction model (as available in GeneSpring or other statistical software packages) to make predictions as described in this example

Class Prediction: The Predict Parameter Values tool in GeneSpring™ software was used for liver inflammation class prediction. A description of this tool and the statistical procedures used is provided in Example 1.

Training and Test Data Sets: The training and test data sets used are those described in Table 15 of Example 3.

Liver Toxicology Classification: Liver inflammation classifications used are described in Table 1 of Example 1. In this analysis randomized classifications (same number of “negative”, “positive-necrosis”, or “positive-necrosis with inflammation” classifications distributed randomly among the samples) were also used.

Prediction Output and Initial Data Processing: For each gene list prediction used for evaluation a table of data generated by the Predict Parameter Values tool in GeneSpring™ software was saved which provided for each sample in the test set the actual call (“negative”, “positive-necrosis with inflammation”, or “positive-necrosis”), the predicted call (“negative”, “positive-necrosis with inflammation”, or “positive-necrosis”) and the P-value cutoff ratio. This set of data was used to calculate predictive performance measures provided below.

Prediction Measures: Accuracy was calculated as described in Example 2.

Results: Prediction results for 6 hour expression data using genes identified as predictive are presented in Table 19 where comparison of predictive performance for correct and random classification is shown. Referring to Table 19, Gene List* is defined as Combo Gene Lists as in Table 18. ** Overall Accuracy=proportion of the total number of predictions that are correct. Non-calls are counted as incorrect predictions as defined in Materials and Methods. Accuracy was calculated for correct classifications of “negative”, “positive-necrosis with inflammation”, or “positive-necrosis” assigned to the samples and for randomized classifications in the same proportions as the correct classifications. Values presented are the mean accuracy values for 5 training/test sets with minimum and maximum accuracy values.

It is clear from these data that the predictions with accurate classification are much better than predictions with randomized classification. This means that the predictive results are not simply due to chance and large data sets but are due to significant, meaningful predictive association between the gene expression of the predictive genes and the liver inflammation.

### Example 5

Materials and Methods: Database: Compounds and Liver inflammation: Compounds and treatments list used to construct the liver database are given in Table 1 of Example 1. This table also provides the evaluation of the liver inflammation observed in samples collected 72 hours after treatment. The database is described in detail in Example 1. This Example analyzes expression data from samples collected 72 hours after treatment.

Array data, normalization and transformation procedures used were as described in Example 1.

Procedures and methods for obtaining gene lists correlating with histopathology scores were as described in Example 1 with scores as in Example 1, Table 1.

The Predict Parameter Values tool in GeneSpring™ software used for liver inflammation class prediction is described in detail in Material and Methods of Example 1.

Training and Test Data Sets: Data were each separated into 5 training and test sets by randomly distributing the compounds into the sets. This was accomplished by assigning random numbers to lists of compounds that are negative and positive for histopathology, sorting by random number, and then dividing the sorted lists into a specific number of training and test sets. The training and test set assignments are presented in the Table 20.

Liver Toxicology Classification: Liver inflammation classifications were entered for training and test set as a parameter column. Toxicity, as defined by observation of liver necrosis or necrosis with inflammation at 72 hours after treatment, was entered as “negative”, “positive-necrosis”, or “positive-necrosis with inflammation” for each animal in a compound-dose group. Additionally, a parameter column for random histopathology classification was designated. This was done by randomly assigning the same number of “negative”, “positive-necrosis”, or “positive-necrosis with inflammation” calls to the individual animals.

Prediction Output and Initial Data Processing: The “Predict Parameter Value” tool of GeneSpring was used with each of the training and test sets to generate predictions of histopathology classifications of the test sets. The number of k nearest neighbors was optimized to give the highest predictive accuracy. This was done by first running predictions at different nearest neighbors for three of the training and test sets, and then evaluating the overall predictive performance for each number of nearest neighbors. A P-value ratio cutoff of 0.5 was used. The number of genes used to predict was varied with standard numbers of 50, 40, 30, 20, 10, 5, 2 and 1 genes used. For each number of genes the numbers of correct calls, incorrect calls and non-calls were recorded. Non-calls are cases where no prediction was made because the P-value ratio exceeded the specified P-value ratio cutoff. Calculations were made for overall percent correct calls (number of correct classifications/number or samples), percent correct calls of called samples (number of correct classifications/number of samples with calls) and percent of called samples (samples with calls/number of samples).

For each input list and optimal number of predictive genes (lowest number of genes giving a maximum overall percent of correct calls) additional information was recorded that included the list of specific genes in the optimum predictive set.

Results: Expression array data were first examined for the existence of genes whose expression correlated with histopathology scores. Table 1 in Materials and Methods of Example 1 presents a list of the compounds and dose levels along with the liver histopathology classification and histopathology severity scores used for this analysis. For each distance measure the probability was adjusted in increments of 0.05 until at least 50 correlating genes were obtained. Lists of correlating genes were obtained using the distance measures described in Materials and Methods. Example sets of correlating genes are provided in Tables 21-22.

The correlating gene lists as well as the entire array gene list were provided as input lists to the GeneSpring Predict Parameter value tool (described in Materials and Methods) that employs a k nearest neighbor (knn) predictive model. These lists as well as the entire array gene list were used for each of the five training and test sets defined in Materials and Methods generate predictions of histopathology classifications of the test sets. Input genes for the Predict Parameter Value feature included all 700 genes in the GenePix file (the Rat CT Array) as well as smaller lists of genes whose expressions correlated with histopathology by the correlation measures described previously. The number of genes used to predict are varied with standard numbers of 50, 40, 30, 20, 10, 5, 2 and 1 genes used. The specified number of predictive genes was varied to obtain an optimum number of predictive genes.

After this was done for all 5 training and test sets, all gene lists were then merged to create one aggregate list of predictive genes. Each gene on this aggregate list has predictive value for at least one of the training and test sets because it was observed to contribute to an optimum predictivity for a specific training/test set. The aggregate list was subdivided into smaller lists of genes based on the number of times a gene was predictive for an individual training or test set. For example, if 5 training and test sets were used, genes that were predictive in all 5 training and test sets were designated as Combo (combination) 5. Genes that were predictive in only 4 of 5 training and test sets were designated as Combo 4, etc.

A list of predictive genes organized by their occurrence in the separate training and test sets is presented in Table 23. Referring to Table 23, Combination (No. of occurrences) is defined as the number of training/test set gene list occurrences.

### Example 6

### Predictive Properties and Evaluation of Predictive Genes for Liver inflammation from 72 Hour Expression Data

Materials and Methods

Database

The database used was as described in Example 1.

Array Data, Normalization and Transformation: Array data, normalization procedures and transformations used in these analyses are as described in Example 1. Table 30 presents 72 hour gene expression data for the predictive genes. These data can be used with a k nearest neighbor prediction model (as available in GeneSpring or other statistical software packages) to make predictions as described in this example.

Class Prediction: The Predict Parameter Values tool in GeneSpring™ software was used for liver inflammation class prediction. A description of this tool and the statistical procedures used is provided in Example 1.

Training and Test Data Sets: The training and test data sets used are those described in the table of Example 5.

Liver Toxicology Classification: Liver inflammation classifications used are described in Table 1 of Example 1. In this analysis randomized classifications (same number of “negative”, “positive-necrosis with inflammation”, or “positive-necrosis” classifications distributed randomly among the samples) were also used.

Prediction Output and Initial Data Processing: For each gene list prediction used for evaluation a table of data generated by the Predict Parameter Values tool in GeneSpring™ software was saved which provided for each sample in the test set the actual call (“negative”, “positive-necrosis with inflammation”, or “positive-necrosis”), the predicted, call (“negative”, “positive-necrosis with inflammation”, or “positive-necrosis”) and the P-value cutoff ratio. This set of data was used to calculate predictive performance measures provided below. Accuracy was calculated as described in Example 2.PResults: Prediction results for 72 hour expression data using genes identified as predictive are presented in Table 24 in which comparison of predictive performance for correct and random classification is shown. Referring to Table 24, the “Gene List*” is derived from Combo Gene Lists as in Table 23. The “**Overall Accuracy” is defined as the proportion of the total number of predictions that are correct. Non-calls are counted as incorrect predictions as defined in Materials and Methods. Accuracy was calculated for correct classifications of “negative”, “positive-necrosis with inflammation”, or “positive-necrosis” assigned to the samples and for randomized classifications in the same proportions as the correct classifications. Values presented are the mean accuracy values for 5 training/test sets with minimum and maximum accuracy values.

It is clear from these data that the predictions with accurate classification are much better than predictions with randomized classification. This means that the predictive results are not simply due to chance and large data sets but are due to significant, meaningful predictive association between the gene expression of the predictive genes and the liver inflammation.

### Example 7

### Alternate Models for Predicting Liver Inflammation

Predictive Modeling: The predictive task with the liver inflammation gene expression data is a three-class classification problem, where the three classes of possible responses are defined as “positive-necrosis with inflammation”, “positive-necrosis”, or “no histopathology”. This is an uneven class problem in that the class of negative responses is roughly 80 percent of the data or more in the database tested. A discrimination function can be used to classify a training set. This function can be cross-validated with a testing set, often repeatedly to quantify the mean and variation of the classification error. There are numerous common discrimination functions, and a comparative study of the performance of these functions is useful in determining the best classifier. Additional measures can then be used to compare the performance of the classifiers. Since the classes are of significantly uneven sizes, use a geometric mean measure (GMM) can be used to compare models, namely, the square root of the product of the true positives and the true negatives.

Common discrimination methods are Fisher's linear discriminant, quadratic discriminant (mahalanobis distance), k-nearest neighbors (knn), logistic discriminant (MacLachlan, “Discriminant Analysis and Statistical Pattern Recognition”, Wiley Series in Probability and Mathematical Statistics, 1992), classification trees (or more generally known as recursive partitioning) (Breiman et al., “Classification and Regression Trees”, Chapman & Hall, 1984; Clark and Pregibon in “Tree-Based Models” (J. M. Chambers and T. J. Hastie, eds.) Chp. 9, Chapman & Hall Computer Science Series, 1993; Quinlan and Kaufman, “C4.5: Programs for Machine Learning”, 1988), and neural network classifiers (Ripley, “Pattern Recognition and Neural Networks”, Cambridge University Press, 1996). Most are formula-based such as linear and quadratic discriminant, whereas others are rule-based, such as recursive partitioning, or algorithmically based, such as knn. knn is also database dependent in that a database containing training set is needed to perform nearest neighbor search and classification.

Classifier Models: A variety of common classification techniques are available. A simple hybrid classifier could be designed and tested, using the knn results, to transform the knn model into a database independent model. This model is termed a centroid model. The centroid model uses the correctly identified test data results from knn and locates a centroid of the subset of k samples that are of the same class for each correctly identified test sample. The centroid is assigned the correct class, and with new test data, a sample is assigned the class of its nearest centroid.

In addition to the knn and centroid models described above, tree, centroid, logistic, and neural network models could also be employed. The neural network is a simple, feed-forward network, allowing skip layers, and with an entropy fitting criterion.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

TABLE 1
Compounds, Dose Levels, Liver Pathology and Abbreviations in the database

| | | | | Liver | Inflamm. | Liver | Necr. |
|---|---|---|---|---|---|---|---|
| Compound | Dose Level | | Abbrev.* | Inflammation | Score** | Necrosis | Score** |
| 1-naphthylisothiocyanate | 15 | mgkg | ANIT 15 | no | 1 | no | 1 |
| 1-naphthylisothiocyanate | 60 | mgkg | ANIT 60 | yes | 2 | yes | 2 |
| 5-fluorouracil | 13 | mg/kg | 5-FU 13 | no | 1 | no | 1 |
| 5-fluorouracil | 50 | mg/kg | 5-FU 50 | no | 1 | no | 1 |
| acetaminophen | 250 | mg/kg | APAP 250 | no | 1 | no | 1 |
| acetaminophen | 1000 | mg/kg | APAP 1000 | no | 1 | yes | 2 |
| aflatoxin | 1 | mg/kg | AFLB 1 | yes | 4 | yes | 8 |
| amphotericin B | 5 | mg/kg | AMPB 5 | no | 1 | no | 1 |
| amphotericin B | 20 | mg/kg | AMPB 20 | no | 1 | no | 1 |
| azathioprine | 50 | mg/kg | AZA 50 | no | 1 | no | 1 |
| azathioprine | 200 | mg/kg | AZA 200 | no | 1 | no | 1 |
| benzene | 0.25 | ml/kg | BEN 250 | no | 1 | no | 1 |
| benzene | 1 | ml/kg | BEN 1000 | no | 1 | no | 1 |
| benzo[a]pyrene | 30 | mg/kg | BAP 30 | no | 1 | no | 1 |
| bromobenzene | 0.2 | ml/kg | BRB 200 | yes | 2 | yes | 2 |
| bromobenzene | 0.8 | ml/kg | BRB 800 | yes | 3 | yes | 4 |
| busulfan | 14 | mg/kg | BUS 14 | no | 1 | no | 1 |
| cadmium chloride | 1 | mg/kg | CAD 1 | no | 1 | no | 1 |
| cadmium chloride | 2 | mg/kg | CAD 2 | no | 1 | no | 1 |
| cadmium chloride | 4 | mg/kg | CAD 4 | yes | 2 | yes | 3 |
| carbon tetrachloride | 0.25 | ml/kg | CCL4 250 | no | 1 | yes | 3 |
| carbon tetrachloride | 1 | ml/kg | CCL4 1000 | yes | 3 | yes | 6 |
| carmustine | 16 | mg/kg | CAR 16 | no | 1 | no | 1 |
| chloroform | 0.25 | ml/kg | CHCL3 250 | no | 1 | no | 1 |
| chloroform | 0.5 | ml/kg | CHCL3 500 | no | 1 | no | 1 |
| chlorpromazine | 8 | mg/kg | CHLOR 8 | no | 1 | no | 1 |
| chlorpromazine | 30 | mg/kg | CHLOR 30 | no | 1 | no | 1 |
| cisplatin | 2.5 | mg/kg | CIS 2.5 | no | 1 | no | 1 |
| cisplatin | 10 | mg/kg | CIS 10 | no | 1 | no | 1 |
| clofibrate | 75 | mg/kg | CLO 75 | no | 1 | no | 1 |
| clofibrate | 250 | mg/kg | CLO 250 | no | 1 | no | 1 |
| clozapine | 45 | mg/kg | CLOZ 45 | no | 1 | no | 1 |
| clozapine | 180 | mg/kg | CLOZ 180 | no | 1 | no | 1 |
| carboxy methyl cellulose | 30 | mg/kg | CMC 30 | no | 1 | no | 1 |
| cycloheximide | 0.5 | mg/kg | CHEX 0.5 | no | 1 | no | 1 |
| cycloheximide | 2 | mg/kg | CHEX 2 | no | 1 | no | 1 |
| cyclophosphamide | 25 | mg/kg | CPHOS 25 | no | 1 | no | 1 |
| cyclophosphamide | 100 | mg/kg | CPHOS 100 | no | 1 | no | 1 |
| cyclosporin A | 20 | mg/kg | CYCA 20 | no | 1 | no | 1 |
| cyclosporin A | 80 | mg/kg | CYCA 80 | no | 1 | no | 1 |
| dexamethasone | 8 | mg/kg | DEX 8 | no | 1 | no | 1 |
| dexamethasone | 30 | mg/kg | DEX 30 | no | 1 | no | 1 |
| diflunisal | 25 | mg/kg | DIP 25 | no | 1 | no | 1 |
| diflunisal | 100 | mg/kg | DIP 100 | no | 1 | no | 1 |
| dimethylnitrosamine | 20 | mg/kg | DMN 20 | yes | 4 | yes | 9 |
| doxorubicin | 12 | mg/kg | DOX 12 | no | 1 | no | 1 |
| erythromycin estolate | 40 | mg/kg | ERY 40 | no | 1 | no | 1 |
| erythromycin estolate | 160 | mg/kg | ERY 160 | no | 1 | no | 1 |
| estradiol | 0.1 | mg/kg | EST 0.1 | no | 1 | no | 1 |
| estradiol | 0.4 | mg/kg | EST 0.4 | no | 1 | no | 1 |
| ethanol | 2.5 | ml/kg | ETH 2500 | no | 1 | no | 1 |
| gancyclovir | 50 | mg/kg | GAN 50 | no | 1 | no | 1 |
| gancyclovir | 200 | mg/kg | GAN 200 | no | 1 | no | 1 |
| gentamicin | 38 | mg/kg | GEN 38 | no | 1 | no | 1 |
| gentamicin | 150 | mg/kg | GEN 150 | no | 1 | no | 1 |
| hydroxyurea | 250 | mg/kg | HYD 250 | no | 1 | no | 1 |
| hydroxyurea | 1000 | mg/kg | HYD 1000 | no | 1 | no | 1 |
| isoniazid | 50 | mg/kg | ISON 50 | no | 1 | no | 1 |
| isoniazid | 200 | mg/kg | ISON 200 | no | 1 | no | 1 |
| ketoconazole | 20 | mg/kg | KETO 20 | no | 1 | no | 1 |
| ketoconazole | 80 | mg/kg | KETO 80 | no | 1 | no | 1 |
| lipopolysaccharide | 2 | mg/kg | LPS 2 | no | 1 | no | 1 |
| lipopolysaccharide | 8 | mg/kg | LPS 8 | yes | 2 | yes | 6 |
| methotrexate | 1.3 | mg/kg | MET 1.3 | no | 1 | no | 1 |
| methotrexate | 5 | mg/kg | MET 5 | no | 1 | no | 1 |
| naloxone | 45 | ml/kg | NAL 45 | no | 1 | no | 1 |
| naloxone | 180 | mg/kg | NAL 180 | no | 1 | no | 1 |
| phenobarbital | 20 | mg/kg | PBARB 20 | no | 1 | no | 1 |
| phenobarbital | 80 | mg/kg | PBARB 80 | no | 1 | no | 1 |
| phenylhydrazine | 20 | mg/kg | PHEN 20 | no | 1 | no | 1 |
| phenylhydrazine | 80 | mg/kg | PHEN 80 | no | 1 | no | 1 |
| polyethylene glycol | 5 | ml/kg | PEG 5000 | no | 1 | no | 1 |
| puromycin | 38 | mg/kg | PUR 38 | no | 1 | no | 1 |
| puromycin | 150 | mg/kg | PUR 150 | no | 1 | no | 1 |
| quinidine | 25 | mg/kg | QUIN 25 | no | 1 | no | 1 |
| quinidine | 100 | mg/kg | QUIN 100 | no | 1 | no | 1 |
| streptozotocin | 20 | mg/kg | STRZ 20 | no | 1 | no | 1 |
| streptozotocin | 75 | mg/kg | STRZ 75 | no | 1 | no | 1 |
| tamoxifen | 50 | mg/kg | TAM 50 | no | 1 | no | 1 |
| tamoxifen | 200 | mg/kg | TAM 200 | no | 1 | no | 1 |
| tetracycline | 50 | mg/kg | TET 50 | no | 1 | no | 1 |
| tetracycline | 150 | mg/kg | TET 150 | no | 1 | yes | 2 |
| theophylline | 25 | mg/kg | THEO 25 | no | 1 | no | 1 |
| theophylline | 100 | mg/kg | THEO 100 | no | 1 | no | 1 |

TABLE 2
Distribution of Compounds* in Individual Training and
Test Sets for 24 h Liver Inflammation Data
Training and Test Set 1

| | | | | | Test Set 1 |
|---|---|---|---|---|---|
| | Training | Training Set 1 | | | Positive**- |
| Training | Set 1 | Positive**- | | Test Set 1 | Necrosis |
| Set 1 | Positive**- | Necrosis with | Test Set 1 | Positive**- | with |
| Negative** | Necrosis | Inflammation | Negative** | Necrosis | Inflammation |
| BAP-Low⁺ | APAP-High⁺ | BRB-Low⁺ | ISON-Low⁺ | TET-High⁺ | BRB-High⁺ |
| KETO-Low | CCL4-Low | CCL4-High | TAM-Low | | LPS-High |
| DOX-Low | | ANIT-High | CYCA-Low | | |
| STRZ-High | | DMN-High | DIF-Low | | |
| ERY-High | | | CHEX-High | | |
| PEG-Low | | | CMC-Low | | |
| PUR-High | | | HYD-Low | | |
| CHLOR-High | | | ANIT-Low | | |
| HYD-High | | | CHEX-Low | | |
| GEN-High | | | APAP-Low | | |
| BEN-High | | | CHCL3-High | | |
| ETH-Low | | | DIF-High | | |
| DOX-High | | | PHEN-High | | |
| PBARB-High | | | GAN-Low | | |
| BUS-Low | | | CYCA-High | | |
| 5-FU-Hi | | | TAM-High | | |
| MET-Low | | | DEX-High | | |
| EST-High | | | CIS-High | | |
| PHEN-Low | | | PUR-Low | | |
| THEO-Low | | | AMPB-Low | | |
| QUIN-Low | | | CLO-High | | |
| GEN-Low | | | EST-Low | | |
| CIS-Low | | | CLOZ-Low | | |
| CLO-Low | | | CAD-Low | | |
| BUS-High | | | CHLOR-Low | | |
| CAR-Low | | | | | |
| LPS-Low | | | | | |
| CPHOS-High | | | | | |
| THEO-High | | | | | |
| NAL-High | | | | | |
| DEX-Low | | | | | |
| NAL-Low | | | | | |
| AMPB-Hi | | | | | |
| 5-FU-Low | | | | | |
| CAD-High | | | | | |
| ISON-High | | | | | |
| STRZ-Low | | | | | |
| CLOZ-High | | | | | |
| TET-Low | | | | | |
| KETO-High | | | | | |
| PBARB-Low | | | | | |
| CHCL3-Low | | | | | |
| BAP-High | | | | | |
| CPHOS-Low | | | | | |
| MET-High | | | | | |
| QUIN-High | | | | | |
| CAR-High | | | | | |
| ERY-Low | | | | | |
| GAN-High | | | | | |
| BEN-Low | | | | | |

Training and Test Set 2

| | Training | Training Set 2 | | | Test Set 2 |
|---|---|---|---|---|---|
| Training | Set 2 | Positive- | | Test Set 2 | Positive- |
| Set 2 | Positive- | Necrosis with | Test Set 2 | Positive- | Necrosis with |
| Negative | Necrosis | Inflammation | Negative | Necrosis | Inflammation |
| PHEN-Low | APAP-High | DMN-High | PUR-High | CCL4-Low | CCL4-High |
| ISON-High | TET-High | BRB-High | KETO-Low | | ANIT-High |
| PHEN-High | | BRB-Low | CLOZ-Low | | |
| BEN-Low | | LPS-High | ERY-High | | |
| CYCA-Low | | | CAR-High | | |
| KETO-High | | | CAD-High | | |
| CLOZ-High | | | PBARB-High | | |
| PBARB-Low | | | 5-FU-Low | | |
| CMC-Low | | | CAR-Low | | |
| CHLOR-Low | | | DEX-Low | | |
| NAL-Low | | | STRZ-Low | | |
| EST-High | | | CLO-Low | | |
| CHCL3-Low | | | ANIT-Low | | |
| DOX-High | | | THEO-Low | | |
| 5-FU-Hi | | | BAP-High | | |
| CPHOS-Low | | | CYCA-High | | |
| DEX-High | | | MET-Low | | |
| DIF-High | | | THEO-High | | |
| ERY-Low | | | ISON-Low | | |
| APAP-Low | | | MET-High | | |
| CIS-Low | | | CHEX-Low | | |
| CLO-High | | | LPS-Low | | |
| BUS-High | | | GEN-Low | | |
| BUS-Low | | | CHCL3-High | | |
| DOX-Low | | | GEN-High | | |
| DIF-Low | | | | | |
| CAD-Low | | | | | |
| STRZ-High | | | | | |
| HYD-Low | | | | | |
| BAP-Low | | | | | |
| CIS-High | | | | | |
| ETH-Low | | | | | |
| BEN-High | | | | | |
| QUIN-High | | | | | |
| PUR-Low | | | | | |
| HYD-High | | | | | |
| EST-Low | | | | | |
| AMPB-Low | | | | | |
| GAN-Low | | | | | |
| NAL-High | | | | | |
| CHEX-High | | | | | |
| CHLOR-High | | | | | |
| GAN-High | | | | | |
| CPHOS-High | | | | | |
| TAM-Low | | | | | |
| TET-Low | | | | | |
| TAM-High | | | | | |
| AMPB-Hi | | | | | |
| QUIN-Low | | | | | |
| PEG-Low | | | | | |

Training and Test Set 3

| | Training | Training Set 3 | | | Test Set 3 |
|---|---|---|---|---|---|
| Training | Set 3 | Positive- | | Test Set 3 | Positive- |
| Set 3 | Positive- | Necrosis with | Test Set 3 | Positive- | Necrosis with |
| Negative | Necrosis | Inflammation | Negative | Necrosis | Inflammation |
| ERY-High | TET-High | BRB-Low | PUR-High | APAP-High | BRB-High |
| EST-High | CCL4-Low | CCL4-High | CPHOS-Low | | LPS-High |
| ISON-Low | | ANIT-High | BEN-High | | |
| ANIT-Low | | LPS-High | HYD-High | | |
| CLO-Low | | | CMC-Low | | |
| CLOZ-Low | | | CLO-High | | |
| DIF-Low | | | GAN-Low | | |
| CAR-Low | | | DOX-High | | |
| LPS-Low | | | CHEX-Low | | |
| CIS-High | | | THEO-Low | | |
| TAM-High | | | AMPB-Hi | | |
| CYCA-High | | | DOX-Low | | |
| MET-Low | | | CHEX-High | | |
| NAL-Low | | | GEN-High | | |
| CPHOS-High | | | DEX-Low | | |
| CAR-High | | | BUS-High | | |
| HYD-Low | | | PUR-Low | | |
| APAP-Low | | | PBARB-Low | | |
| GEN-Low | | | 5-FU-Low | | |
| AMPB-Low | | | QUIN-Low | | |
| PHEN-Low | | | STRZ-Low | | |
| BAP-High | | | ISON-High | | |
| EST-Low | | | ETH-Low | | |
| CHCL3-High | | | STRZ-High | | |
| CAD-High | | | DEX-High | | |
| PHEN-High | | | | | |
| TET-Low | | | | | |
| CLOZ-High | | | | | |
| BEN-Low | | | | | |
| CHLOR-High | | | | | |
| TAM-Low | | | | | |
| DIF-High | | | | | |
| BUS-Low | | | | | |
| KETO-High | | | | | |
| 5-FU-Hi | | | | | |
| MET-High | | | | | |
| ERY-Low | | | | | |
| QUIN-High | | | | | |
| BAP-Low | | | | | |
| KETO-Low | | | | | |
| THEO-High | | | | | |
| PBARB-High | | | | | |
| CYCA-Low | | | | | |
| NAL-High | | | | | |
| CIS-Low | | | | | |
| PEG-Low | | | | | |
| CHLOR-Low | | | | | |
| GAN-High | | | | | |
| CHCL3-Low | | | | | |
| CAD-Low | | | | | |

Training and Test Set 4

| | Training | Training Set 4 | | | Test Set 4 |
|---|---|---|---|---|---|
| Training | Set 4 | Positive- | | Test Set 4 | Positive- |
| Set 4 | Positive- | Necrosis with | Test Set 4 | Positive- | Necrosis with |
| Negative | Necrosis | Inflammation | Negative | Necrosis | Inflammation |
| CHEX-Low | APAP-High | LPS-High | AMPB-Low | TET-High | BRB-High |
| 5-FU-Low | TET-High | DMN-High | PHEN-Low | | LPS-High |
| BEN-High | | ANIT-High | DIF-Low | | |
| QUIN-Low | | BRB-Low | APAP-Low | | |
| ERY-Low | | | CAD-High | | |
| ETH-Low | | | GAN-Low | | |
| CYCA-High | | | HYD-High | | |
| KETO-High | | | TAM-High | | |
| GEN-Low | | | DOX-Low | | |
| BAP-High | | | GEN-High | | |
| PEG-Low | | | PHEN-High | | |
| BAP-Low | | | TET-Low | | |
| CMC-Low | | | MET-High | | |
| BUS-High | | | CHEX-High | | |
| BUS-Low | | | DOX-High | | |
| THEO-High | | | STRZ-High | | |
| CYCA-Low | | | PBARB-High | | |
| DEX-High | | | CLO-High | | |
| QUIN-High | | | KETO-Low | | |
| ERY-High | | | BEN-Low | | |
| DEX-Low | | | 5-FU-Hi | | |
| EST-High | | | ISON-Low | | |
| CAR-High | | | CAD-Low | | |
| CHLOR-Low | | | CIS-Low | | |
| MET-Low | | | PUR-High | | |
| CHLOR-High | | | | | |
| CAR-Low | | | | | |
| AMPB-Hi | | | | | |
| CPHOS-High | | | | | |
| CLO-Low | | | | | |
| NAL-Low | | | | | |
| HYD-Low | | | | | |
| ANIT-Low | | | | | |
| ISON-High | | | | | |
| EST-Low | | | | | |
| CIS-High | | | | | |
| CHCL3-High | | | | | |
| NAL-High | | | | | |
| GAN-High | | | | | |
| CLOZ-High | | | | | |
| LPS-Low | | | | | |
| CLOZ-Low | | | | | |
| THEO-Low | | | | | |
| CPHOS-Low | | | | | |
| PUR-Low | | | | | |
| TAM-Low | | | | | |
| DIF-High | | | | | |
| PBARB-Low | | | | | |
| CHCL3-Low | | | | | |
| STRZ-Low | | | | | |

Training and Test Set 5

| | Training | Training Set 5 | | | Test Set 5 |
|---|---|---|---|---|---|
| Training | Set 5 | Positive- | | Test Set 5 | Positive- |
| Set 5 | Positive- | Necrosis with | Test Set 5 | Positive- | Necrosis with |
| Negative | Necrosis | Inflammation | Negative | Necrosis | Inflammation |
| KETO-High | APAP-High | CCL4-High | ISON-Low | TET-High | LPS-High |
| 5-FU-Hi | CCL4-Low | BRB-High | MET-Low | | BRB-Low |
| CIS-Low | | ANIT-High | CHCL3-High | | |
| NAL-Low | | DMN-High | PHEN-High | | |
| GAN-High | | | TAM-Low | | |
| CPHOS-High | | | GEN-Low | | |
| CHCL3-Low | | | CLO-Low | | |
| CHEX-Low | | | MET-High | | |
| PUR-Low | | | QUIN-Low | | |
| AMPB-Hi | | | STRZ-High | | |
| PEG-Low | | | KETO-Low | | |
| TET-Low | | | DEX-High | | |
| CYCA-Low | | | CAD-Low | | |
| DOX-Low | | | BUS-Low | | |
| ETH-Low | | | EST-Low | | |
| HYD-Low | | | BEN-Low | | |
| STRZ-Low | | | CAD-High | | |
| EST-High | | | CAR-High | | |
| CHLOR-High | | | CIS-High | | |
| 5-FU-Low | | | CHLOR-Low | | |
| LPS-Low | | | APAP-Low | | |
| THEO-Low | | | DIF-High | | |
| NAL-High | | | CLOZ-Low | | |
| DOX-High | | | PBARB-High | | |
| PBARB-Low | | | CPHOS-Low | | |
| DIF-Low | | | | | |
| ERY-High | | | | | |
| QUIN-High | | | | | |
| ERY-Low | | | | | |
| CMC-Low | | | | | |
| ISON-High | | | | | |
| CLOZ-High | | | | | |
| BEN-High | | | | | |
| CHEX-High | | | | | |
| PHEN-Low | | | | | |
| ANIT-Low | | | | | |
| CLO-High | | | | | |
| THEO-High | | | | | |
| PUR-High | | | | | |
| BAP-Low | | | | | |
| CAR-Low | | | | | |
| DEX-Low | | | | | |
| GEN-High | | | | | |
| BAP-High | | | | | |
| HYD-High | | | | | |
| BUS-High | | | | | |
| GAN-Low | | | | | |
| AMPB-Low | | | | | |
| CYCA-High | | | | | |
| TAM-High | | | | | |

TABLE 3
List of Genes, Whose Expression at 24 h Directly
Correlates with Liver Inflammation at 72 h, Ranked
by Pearson Correlation Coefficient

| | Correlation |
|---|---|
| Gene | Coefficient |
| Phase-1 RCT-207 | 0.598 |
| Zinc finger protein | 0.592 |
| Gadd45 | 0.578 |
| Gamma-actin, cytoplasmic | 0.566 |
| Heme oxygenase | 0.558 |
| Phase-1 RCT-50 | 0.549 |
| Phase-1 RCT-144 | 0.547 |
| Phase-1 RCT-179 | 0.546 |
| Macrophage inflammatory protein-2 alpha | 0.545 |
| Superoxide dismutase Mn | 0.533 |
| Multidrug resistant protein-2 | 0.527 |
| Phase-1 RCT-225 | 0.524 |
| 14-3-3 zeta | 0.518 |
| Cyclin G | 0.507 |
| Cofilin | 0.502 |
| Gadd153 | 0.501 |
| Phase-1 RCT-242 | 0.492 |
| c-jun | 0.490 |
| Cathepsin L, sequence 2 | 0.488 |
| Phase-1 RCT-68 | 0.479 |
| Phase-1 RCT-39 | 0.469 |
| ID-1 | 0.464 |
| Calpactin I heavy chain | 0.463 |
| PAR interacting protein | 0.453 |
| Endogenous retroviral sequence, 5′ and 3′ LTR | 0.446 |
| IkB-a | 0.441 |
| Phase-1 RCT-59 | 0.440 |
| Phase-1 RCT-158 | 0.438 |
| Phase-1 RCT-109 | 0.436 |
| Multidrug resistant protein-1 | 0.431 |
| Phase-1 RCT-205 | 0.430 |
| Phase-1 RCT-49 | 0.429 |
| Phase-1 RCT-145 | 0.425 |
| Phase-1 RCT-213 | 0.425 |
| Phase-1 RCT-72 | 0.419 |
| 60S ribosomal protein L6 | 0.415 |
| Voltage-dependent anion channel 2 (Vdac2) | 0.411 |
| Phase-1 RCT-152 | 0.407 |
| 60S ribosomal protein L6 (alternate clone 1) | 0.407 |
| c-myc | 0.406 |
| Ribosomal protein L13A | 0.406 |
| IgE binding protein | 0.406 |
| Melanoma-associated antigen ME491 | 0.405 |
| Beta-actin | 0.403 |
| c-H-ras | 0.399 |
| Phase-1 RCT-154 | 0.399 |
| Phase-1 RCT-122 | 0.398 |
| Integrin beta1 | 0.397 |
| Ornithine decarboxylase | 0.395 |
| Beta-tubulin, class I | 0.395 |
| Phase-1 RCT-241 | 0.395 |
| Retinoid X receptor alpha | 0.394 |
| Bax (alpha) | 0.394 |
| Caspase 3 | 0.388 |
| Insulin-like growth factor binding protein 1 | 0.385 |
| Nucleoside diphosphate kinase beta isoform | 0.385 |
| Phase-1 RCT-60 | 0.384 |
| Phase-1 RCT-196 | 0.382 |
| Phase-1 RCT-192 | 0.380 |
| Organic cation transporter 3 | 0.379 |
| Thymosin beta-10 | 0.379 |
| Osteoactivin | 0.379 |
| Phase-1 RCT-12 | 0.375 |
| Phase-1 RCT-65 | 0.363 |
| Waf1 | 0.360 |
| Alpha-tubulin | 0.360 |
| Phase-1 RCT-215 | 0.359 |
| Carbonyl reductase | 0.359 |
| p53 | 0.356 |
| Phase-1 RCT-71 | 0.355 |
| Phase-1 RCT-191 | 0.353 |
| Beta-actin, sequence 2 | 0.352 |
| Uncoupling protein 2 | 0.350 |

TABLE 4
List of Genes, Whose Expression at 24 h Inversely
Correlates with Liver Inflammation at 72 h, Ranked
by Spearman Correlation Coefficient

| | Correlation |
|---|---|
| Gene | Coefficient |
| Matrin F/G | −0.425 |
| Phase-1 RCT-36 | −0.415 |
| Phase-1 RCT-78 | −0.403 |
| Phase-1 RCT-33 | −0.403 |
| Phase-1 RCT-38 | −0.402 |
| Hepatic lipase | −0.399 |
| Phase-1 RCT-214 | −0.397 |
| Carbonic anhydrase III | −0.394 |
| Phase-1 RCT-288 | −0.393 |
| L-gulono-gamma-lactone oxidase | −0.393 |
| Phase-1 RCT-92 | −0.392 |
| Phase-1 RCT-256 | −0.391 |
| Sodium/bile acid co-transporter | −0.382 |
| Alpha 1 - inhibitor III | −0.380 |
| Phase-1 RCT-89 | −0.380 |
| Liver fatty acid binding protein | −0.379 |
| Phase-1 RCT-296 | −0.376 |
| Organic anion transporter 3 | −0.376 |
| Phase-1 RCT-291 | −0.375 |
| Dynamin-1 (D100) | −0.375 |
| Presenilin-1 | −0.373 |
| Aldehyde dehydrogenase, microsomal | −0.370 |
| Phase-1 RCT-102 | −0.365 |
| Equilbrative nitrobenzylthioinosine- | −0.364 |
| sensitive nucleoside transporter | |
| Phase-1 RCT-52 | −0.363 |
| Phase-1 RCT-168 | −0.362 |
| Sterol carrier protein 2 | −0.362 |
| N-hydroxy-2-acetylaminofluorene | −0.359 |
| sulfotransferase (ST1C1) | |
| Phase-1 RCT-218 | −0.359 |
| Senescence marker protein-30 | −0.357 |
| Phase-1 RCT-40 | −0.352 |
| Paraoxonase 1 | −0.352 |
| Tryptophan hydroxylase | −0.351 |
| Phase-1 RCT-123 | −0.348 |
| Phase-1 RCT-83 | −0.347 |
| Transthyretin | −0.347 |
| Phase-1 RCT-219 | −0.345 |
| Phase-1 RCT-88 | −0.341 |
| Phase-1 RCT-289 | −0.341 |
| Apolipoprotein CIII | −0.341 |
| Phase-1 RCT 165 | −0.337 |
| Phase-1 RCT-128 | −0.336 |
| Phase-1 RCT-264 | −0.335 |
| Phase-1 RCT-64 | −0.335 |
| Phase-1 RCT-233 | −0.334 |
| Phase-1 RCT-181 | −0.333 |
| Aquaporin-3 (AQP3) | −0.332 |
| Phase-1 RCT-175 | −0.331 |
| Cytochrome P450 2C23 | −0.330 |
| Urinary protein 2 precursor | −0.327 |
| 3-hydroxyisobutyrate dehydrogenase | −0.327 |
| Phase-1 RCT-117 | −0.326 |
| Glutathione peroxidase | −0.324 |
| Phase-1 RCT-182 | −0.324 |
| Fatty acid synthase | −0.322 |
| Phase-1 RCT-271 | −0.321 |
| Phase-1 RCT-10 | −0.321 |
| Phase-1 RCT-209 | −0.320 |
| Phase- 1 RCT-67 | −0.320 |
| HMG-CoA synthase, mitochondrial | −0.316 |
| Phase-1 RCT-137 | −0.315 |
| Stearyl-CoA desaturase, liver | −0.314 |
| Apoptpsis-regulating basic protein | −0.312 |
| Phase-1 RCT-185 | −0.312 |
| Phase-1 RCT-98 | −0.312 |
| Phase-1 RCT-239 | −0.312 |
| Carbonic anhydrase III, sequence 2 | −0.308 |
| Phase-1 RCT-189 | −0.308 |
| Phase-1 RCT-270 | −0.308 |
| NADH-cytochrome b5 reductase | −0.308 |
| Sulfotransferase K2 | −0.301 |

TABLE 5
Predictive Genes for 24 Hour Expression Data

| | Combination |
|---|---|
| Gene Name | Category* |
| Gamma-actin, cytoplasmic | 5 |
| 60S ribosomal protein L6 (alternate clone 1) | 3 |
| 60S ribosomal protein L6 | 3 |
| Beta-tubulin, class I | 3 |
| c-jun | 3 |
| Gadd45 | 3 |
| ID-1 | 3 |
| IkB-a | 3 |
| Integrin beta1 | 3 |
| Macrophage inflammatory protein-2 alpha | 3 |
| MAP kinase kinase | 3 |
| Multidrug resistant protein-2 | 3 |
| Organic cation transporter 3 | 3 |
| Phase-1 RCT-144 | 3 |
| Phase-1 RCT-145 | 3 |
| Phase-1 RCT-179 | 3 |
| Phase-1 RCT-192 | 3 |
| Phase-1 RCT-207 | 3 |
| Phase-1 RCT-225 | 3 |
| Phase-1 RCT-242 | 3 |
| Phase-1 RCT-49 | 3 |
| Phase-1 RCT-50 | 3 |
| Phase-1 RCT-92 | 3 |
| Zinc finger protein | 3 |
| 14-3-3 zeta | 2 |
| Alpha-tubulin | 2 |
| Beta-actin | 2 |
| Cathepsin L, sequence 2 | 2 |
| c-myc | 2 |
| Cytochrome P450 11A1 | 2 |
| Gadd153 | 2 |
| IgE binding protein | 2 |
| L-gulono-gamma-lactone oxidase | 2 |
| Matrin F/G | 2 |
| MHC class I antigen RT1.A1(f) alpha-chain | 2 |
| Nucleoside diphosphate kinase beta isoform | 2 |
| Ornithine decarboxylase | 2 |
| PAR interacting protein | 2 |
| Phase-1 RCT-181 | 2 |
| Phase-1 RCT-185 | 2 |
| Phase-1 RCT-205 | 2 |
| Phase-1 RCT-213 | 2 |
| Phase-1 RCT-233 | 2 |
| Phase-1 RCT-258 | 2 |
| Phase-1 RCT-288 | 2 |
| Phase-1 RCT-33 | 2 |
| Phase-1 RCT-36 | 2 |
| Phase-1 RCT-39 | 2 |
| Phase-1 RCT-60 | 2 |
| Phase-1 RCT-64 | 2 |
| Phase-1 RCT-65 | 2 |
| Phase-1 RCT-78 | 2 |
| Phase-1 RCT-98 | 1 |
| Aldehyde dehydrogenase, microsomal | 1 |
| Alpha 1 - inhibitor III | 1 |
| Alpha-2-microglobulin | 1 |
| Apolipoprotein AII | 1 |
| Apolipoprotein CIII | 1 |
| Aquaporin-3 (AQP3) | 1 |
| Argininosuccinate lyase | 1 |
| Aspartate aminotransferase, mitochondrial | 1 |
| Urinary protein 2 precursor | 1 |
| ATP-stimulated glucocorticoid-receptor | 1 |
| translocation promoter (Gyk) | |
| Bax (alpha) | 1 |
| Beta-actin, sequence 2 | 1 |
| Beta-alanine synthase | 1 |
| Carbonic anhydrase III | 1 |
| Carbonic anhydrase III, sequence 2 | 1 |
| Carbonyl reductase | 1 |
| Carnitine palmitoyl-CoA transferase | 1 |
| Casein-alpha | 1 |
| Caspase 3 | 1 |
| CDK102 | 1 |
| c-H-ras | 1 |
| Cofilin | 1 |
| Cyclin D1 | 1 |
| Cyclin G | 1 |
| Cytochrome P450 2C23 | 1 |
| Dynamin-1 (D100) | 1 |
| Elongation factor-1 alpha | 1 |
| Endogenous retroviral sequence, 5′ and 3′ LTR | 1 |
| Endothelin-1 | 1 |
| Equilbrative nitrobenzylthioinosine-sensitive | 1 |
| nucleoside transporter | |
| Fas antigen | 1 |
| Glutathione peroxidase | 1 |
| Heme oxygenase | 1 |
| Hepatic lipase | 1 |
| Hepatocyte growth factor receptor | 1 |
| HMG-CoA synthase, mitochondrial | 1 |
| Insulin-like growth factor binding protein 1 | 1 |
| Interleukin-10 | 1 |
| Liver fatty acid binding protein | 1 |
| Malic enzyme | 1 |
| Melanoma-associated antigen ME491 | 1 |
| Multidrug resistant protein-1 | 1 |
| MutL homologue (MLH1) | 1 |
| NADH-cytochrome b5 reductase | 1 |
| NADP-dependent isocitrate dehydrogenase, cytosolic | 1 |
| N-hydroxy-2-acetylaminofluorene | 1 |
| sulfotransferase (ST1C1) | |
| Octamer binding protein 1 | 1 |
| Organic anion transporter 3 | 1 |
| p53 | 1 |
| Paraoxonase 1 | 1 |
| Phase-1 RCT-10 | 1 |
| Phase-1 RCT-102 | 1 |
| Phase-1 RCT-109 | 1 |
| Phase-1 RCT-111 | 1 |
| Phase-1 RCT-113 | 1 |
| Phase-1 RCT-115 | 1 |
| Phase-1 RCT-117 | 1 |
| Phase-1 RCT-12 | 1 |
| Phase-1 RCT-123 | 1 |
| Phase-1 RCT-128 | 1 |
| Apoptosis-regulating basic protein | 1 |
| Phase-1 RCT-137 | 1 |
| Phase-1 RCT-140 | 1 |
| Phase-1 RCT-141 | 1 |
| Phase-1 RCT-152 | 1 |
| Phase-1 RCT-154 | 1 |
| Phase-1 RCT-158 | 1 |
| Phase-1 RCT-168 | 1 |
| Phase-1 RCT-174 | 1 |
| Phase-1 RCT-175 | 1 |
| Phase-1 RCT-180 | 1 |
| Phase-1 RCT-182 | 1 |
| Phase-1 RCT-189 | 1 |
| Phase-1 RCT-191 | 1 |
| Phase-1 RCT-196 | 1 |
| Vacuole membrane protein 1 | 1 |
| Phase-1 RCT-209 | 1 |
| Phase-1 RCT-211 | 1 |
| Phase-1 RCT-212 | 1 |
| Phase-1 RCT-214 | 1 |
| Phase-1 RCT-215 | 1 |
| Phase-1 RCT-218 | 1 |
| Phase-1 RCT-219 | 1 |
| Phase-1 RCT-239 | 1 |
| Phase-1 RCT-24 | 1 |
| Phase-1 RCT-241 | 1 |
| Phase-1 RCT-256 | 1 |
| Phase-1 RCT-264 | 1 |
| Phase-1 RCT-27 | 1 |
| Phase-1 RCT-270 | 1 |
| Phase-1 RCT-271 | 1 |
| Phase-1 RCT-281 | 1 |
| Phase-1 RCT-282 | 1 |
| Phase-1 RCT-287 | 1 |
| Phase-1 RCT-289 | 1 |
| Phase-1 RCT-291 | 1 |
| Voltage-dependent anion channel 2 (Vdac2) | 1 |
| Phase-1 RCT-296 | 1 |
| Phase-1 RCT-30 | 1 |
| Phase-1 RCT-37 | 1 |
| Phase-1 RCT-38 | 1 |
| Phase-1 RCT-40 | 1 |
| Phase-1 RCT-48 | 1 |
| Phase-1 RCT-52 | 1 |
| Phase-1 RCT-67 | 1 |
| Phase-1 RCT-68 | 1 |
| Phase-1 RCT-72 | 1 |
| Phase-1 RCT-76 | 1 |
| Phase-1 RCT-77 | 1 |
| Phase-1 RCT-79 | 1 |
| Phase-1 RCT-8 | 1 |
| Phase-1 RCT-88 | 1 |
| Phase-1 RCT-89 | 1 |
| Preproalbumin, sequence 2 | 1 |
| Presenilin-1 | 1 |
| Pyruvate kinase, muscle | 1 |
| Retinol-binding protein (RBP) | 1 |
| Ribosomal protein L13A | 1 |
| Ribosomal protein S9 | 1 |
| Senescence marker protein-30 | 1 |
| Sodium/bile acid cotransporter | 1 |
| Sodium/glucose cotransporter 1 | 1 |
| Sorbitol dehydrogenase | 1 |
| Stearyl-CoA desaturase, liver | 1 |
| Sterol carrier protein 2 | 1 |
| Sulfotransferase K2 | 1 |
| Superoxide dismutase Mn | 1 |
| Thymosin beta-10 | 1 |
| Transthyretin | 1 |
| Tryptophan hydroxylase | 1 |

TABLE 6
Randomly Selected Gene Subsets from
24 H Combo All (183 Genes)*

| Rand 5 (1) | Rand 5 (2) |
|---|---|
| Aquaporin-3 (AQP3) | Apolipoprotein CIII |
| Phase-1 RCT-115 | Cofilin |
| Phase-1 RCT-209 | Voltage-dependent anion |
| | channel 2 (Vdac2) |
| Pyruvate kinase, muscle | Phase-1 RCT-271 |
| Transthyretin | Phase-1 RCT-196 |
| Rand 10 (1) | Rand 10 (2) |
| Aspartate aminotransferase, | PAR interacting protein |
| mitochondrial | |
| Casein-alpha | Phase-1 RCT-38 |
| Fas antigen | Integrin beta1 |
| Gadd45 | Phase-1 RCT-141 |
| Gamma-actin, cytoplasmic | Phase-1 RCT-50 |
| Integrin beta1 | Liver fatty acid binding protein |
| Macrophage inflammatory | Beta-actin, sequence 2 |
| protein-2 alpha | |
| Phase-1 RCT-145 | 60S ribosomal protein L6 |
| Phase-1 RCT-207 | Phase-1 RCT-211 |
| Phase-1 RCT-78 | Ribosomal protein L13A |
| Rand 15 (1) | Rand 15 (2) |
| 60S ribosomal protein | Phase-1 RCT-52 |
| L6 (alternate clone 1) | |
| Argininosuccinate lyase | HMG-CoA synthase, mitochondrial |
| Cytochrome P450 11A1 | Retinol-binding protein (RBP) |
| Dynamin-1 (D100) | Sodium/bile acid cotransporter |
| Endogenous retroviral | Beta-alanine synthase |
| sequence, 5′ and 3′ | |
| LTR | |
| Integrin beta1 | Ornithine decarboxylase |
| Paraoxonase 1 | Insulin-like growth factor |
| | binding protein 1 |
| Apoptosis-regulating basic | Phase-1 RCT-109 |
| protein | |
| Phase-1 RCT-181 | Octamer binding protein 1 |
| Phase-1 RCT-264 | Phase-1 RCT-145 |
| Voltage-dependent anion | NADP-dependent isocitrate |
| channel 2 (Vdac2) | dehydrogenase, cytosolic |
| Phase-1 RCT-33 | Phase-1 RCT-39 |
| Phase-1 RCT-36 | Matrin F/G |
| Phase-1 RCT-52 | Phase-1 RCT-289 |
| Thymosin beta-10 | Organic anion transporter 3 |

TABLE 7
Randomly Selected Gene Subsets from 24
H Combo 5 3 2 Gene Set (52 Genes)*

| Rand 5 (1) | Rand 5 (2) |
|---|---|
| Phase-1 RCT-207 | Phase-1 RCT-233 |
| 60S ribosomal protein | Integrin beta1 |
| L6 (alternate clone 1) | |
| Cathepsin L | Phase-1 RCT-50 |
| Phase-1 RCT-145 | Phase-1 RCT-145 |
| Phase-1 RCT-65 | Phase-1 RCT-225 |
| Rand 10 (1) | Rand 10 (2) |
| MHC class 1 antigen RT1.A1(f) | Phase-1 RCT-65 |
| alpha-chain | |
| Beta-actin | Gadd153 |
| Beta-tubulin, class I | Phase-1 RCT-36 |
| Cathepsin L | Phase-1 RCT-60 |
| c-jun | Phase-1 RCT-181 |
| Matrin F/G | 60S ribosomal protein L6 |
| Phase-1 RCT-225 | Phase-1 RCT-144 |
| Phase-1 RCT-288 | Phase-1 RCT-192 |
| Phase-1 RCT-36 | Zinc finger protein |
| Phase-1 RCT-50 | Phase-1 RCT-205 |
| Rand 15 (1) | Rand 15 (2) |
| Phase-1 RCT-242 | 60S ribosomal protein L6 (alternate |
| | clone 1) |
| IkB-a | 14-3-3 zeta |
| MAP kinase kinase | 60S ribosomal protein L6. |
| Matrin F/G | Alpha-tubulin |
| Multidrug resistant protein-2 | Beta-actin |
| Nucleoside diphosphate kinase | Beta-tubulin, class I |
| beta isoform | |
| Organic cation transporter 3 | Cathepsin L |
| PAR interacting protein | c-jun |
| Phase-1 RCT-179 | c-myc |
| Phase-1 RCT-288 | Cytochrome P450 11A1 |
| Phase-1 RCT-33 | Gadd153 |
| Phase-1 RCT-36 | Gadd45 |
| Phase-1 RCT-39 | Gamma-actin, cytoplasmic |
| Phase-1 RCT-64 | ID-1 |
| Phase-1 RCT-92 | IgE binding protein |

TABLE 8
Randomly Selected Gene Subsets from
Array Genes Excluding Combo All Set*

| Rand 5 (1) | Rand 5 (2) |
|---|---|
| Heme binding protein 23 | Phase-1 RCT-147 |
| alpha-1,2-fucosyltransferase | NADPH cytochrome P450 reductase |
| Metallothionein 1 | Phase-1 RCT-236 |
| Phase-1 RCT-83 | CXCR4 |
| Pim1 proto-oncogene | TGF-beta receptor type II |
| Rand 10 (1) | Rand 10 (2) |
| Protein kinase C beta1 | Phase-1 RCT-176 |
| Phase-1 RCT-14 | p55CDC |
| Retinoid X receptor alpha | Connexin-32 |
| Phase-1 RCT-221 | Aryl sulfotransferase |
| Cytochrome P450 2C11 | Diacylglycerol kinase zeta |
| Phase-1 RCT-173 | Phase-1 RCT-59 |
| Inter-alpha-inhibitor H4 | Phase-1 RCT-293 |
| heavy chain (Itih4) | |
| Major acute phase | Thioredoxin-2 (Trx2) |
| protein alpha-1 | |
| ADP-ribosylation factor- | Diazepam binding inhibitor |
| like protein ARL184 | |
| Cellular retinoic acid binding | Phase-1 RCT-47 |
| protein 2 | |
| Rand 15 (1) | Rand 15 (2) |
| Phase-1 RCT-42 | Neurofibromin (NF1 tumor suppressor) |
| Tissue factor pathway inhibitor | Interleukin-1 beta |
| C-reactive protein | Glutathione S-transferase alpha subunit |
| Caspase 2 | Protein O-mannosyltransferase 1 |
| | (Pomt1) |
| Cyclin D3 | Phase-1 RCT-32 |
| Dopamine transporter | Monoamine oxidase A |
| DNA topoisomerase I | 25-hydroxyvitamin D3-1 alpha- |
| | hydroxylase |
| Multidrug resistant protein-3 | Acyl-CoA dehydrogenase, medium |
| | chain |
| Defender against cell death-1 | Macrophage inflammatory protein-1 |
| | alpha |
| CXCR4 | Phase-1 RCT-133 |
| Cytochrome c oxidase subunit II | Na/K ATPase alpha-1 |
| Low density lipoprotein receptor | Vesicular monoamine transporter |
| | (VMAT) |
| Farnesol receptor | Phase-1 RCT-176 |
| H-rev107 | Alpha-fetoprotein |
| 8-oxoguanine DNA glycosylase | Phase-1 RCT-177 |

TABLE 9
Liver Inflammation Individual Sample Prediction Values for
24 Hour Data Predictive Genes (Combined List and Subsets)

| Gene | Prediction Measure* | | | | |
|---|---|---|---|---|---|
| Set | Overall | | | | |
| (#) | Accuracy** | FP_{I}** | FN_{I}** | GMM_{I}** | GMM_{N}** |
| Combo | 0.860 | 0.092 | 0.167 | 0.862 | 0.891 |
| All | (0.785- | (0.014- | (0.000- | (0.671- | (0.791- |
| (183) | 0.933) | 0.123) | 0.500) | 0.993) | 0.939) |
| Combo | 0.845 | 0.120 | 0.100 | 0.890 | 0.845 |
| 5 | (0.779- | (0.075- | (0.000- | (0.832- | (0.777- |
| (1) | 0.904) | 0.169) | 0.167) | 0.962) | 0.905) |
| Combo | 0.849 | 0.098 | 0.167 | 0.861 | 0.823 |
| 3 | (0.831- | (0.029- | (0.000- | (0.765- | (0.555- |
| (23) | 0.880) | 0.152) | 0.333) | 0.954) | 0.919) |
| Combo | 0.793 | 0.171 | 0.300 | 0.753 | 0.857 |
| 2 | (0.747- | (0.116- | (0.000- | (0.636- | (0.759- |
| (28) | 0.827) | 0.212) | 0.500) | 0.888) | 0.893) |
| Combo | 0.804 | 0.156 | 0.200 | 0.817 | 0.860 |
| 1 | (0.709- | (0.043- | (0.000- | (0.645- | (0.729- |
| (131) | 0.907) | 0.205) | 0.500) | 0.978) | 0.945) |

TABLE 10
Liver Inflammation Compound-Dose Prediction Values for
24 Hour Data Predictive Genes (Combined List and Subsets)

| | Number | |
|---|---|---|
| Gene Set | of Genes | Overall Accuracy** |
| Combo | 183 | 0.869 (0.741-0.962) |
| All | | |
| Combo 5 | 1 | 0.892 (0.846-0.958) |
| Combo 3 | 23 | 0.860 (0.833-0.885) |
| Combo 2 | 28 | 0.814 (0.769-0.846) |
| Combo 1 | 131 | 0.839 (0.704-0.885) |

TABLE 11
Liver Inflammation Compound Prediction Values for
24 Hour Data Predictive Genes (Combined List and Subsets)

| | Number | |
|---|---|---|
| Gene Set | of Genes | Overall Accuracy** |
| Combo | 183 | 0.864 (0.739-0.955) |
| All | | |
| Combo 5 | 1 | 0.886 (0.826-0.952) |
| Combo 3 | 23 | 0.855 (0.810-0.885) |
| Combo 2 | 28 | 0.796 (0.739-0.846) |
| Combo 1 | 131 | 0.839 (0.696-0.909) |

TABLE 12
Individual Gene Predictions: Combo 3

| | Overall Correct | | | |
|---|---|---|---|---|
| | Calls | | | |
| Gene Name | Mean | s.d. | min | max |
| 60S ribosomal protein L6 (alternate clone 1) | 0.602 | 0.084 | 0.493 | 0.708 |
| 60S ribosomal protein L6 | 0.715 | 0.024 | 0.693 | 0.753 |
| Beta-tubulin, class I | 0.417 | 0.042 | 0.356 | 0.468 |
| c-jun | 0.641 | 0.044 | 0.573 | 0.685 |
| Gadd45 | 0.727 | 0.063 | 0.667 | 0.805 |
| ID-1 | 0.564 | 0.053 | 0.519 | 0.640 |
| IkB-a | 0.629 | 0.070 | 0.557 | 0.720 |
| Integrin beta1 | 0.740 | 0.061 | 0.688 | 0.840 |
| MAP kinase kinase | 0.570 | 0.070 | 0.506 | 0.667 |
| Macrophage inflammatory protein-2 alpha | 0.561 | 0.058 | 0.479 | 0.640 |
| Multidrug resistant protein-2 | 0.609 | 0.082 | 0.542 | 0.709 |
| Organic cation transporter 3 | 0.711 | 0.070 | 0.611 | 0.805 |
| Phase-1 RCT-144 | 0.762 | 0.052 | 0.722 | 0.844 |
| Phase-1 RCT-145 | 0.634 | 0.128 | 0.452 | 0.779 |
| Phase-1 RCT-179 | 0.710 | 0.038 | 0.658 | 0.764 |
| Phase-1 RCT-192 | 0.675 | 0.051 | 0.625 | 0.760 |
| Phase-1 RCT-207 | 0.734 | 0.022 | 0.696 | 0.753 |
| Phase-1 RCT-225 | 0.579 | 0.023 | 0.556 | 0.608 |
| Phase-1 RCT-242 | 0.621 | 0.106 | 0.468 | 0.747 |
| Phase-1 RCT-49 | 0.665 | 0.057 | 0.587 | 0.727 |
| Phase-1 RCT-50 | 0.609 | 0.032 | 0.575 | 0.653 |
| Phase-1 RCT-92 | 0.604 | 0.335 | 0.231 | 0.883 |
| Zinc finger protein | 0.775 | 0.041 | 0.720 | 0.819 |
| Average Individual Combo 3 | 0.646 | 0.070 | 0.564 | 0.729 |
| Minimum Individual Combo 3 | 0.417 | 0.022 | 0.231 | 0.468 |
| Maximum Individual Combo 3 | 0.775 | 0.335 | 0.722 | 0.883 |

TABLE 13
Individual Gene Predictions: Combo 2

| | Overall Correct | | | |
|---|---|---|---|---|
| | Calls | | | |
| Gene Name | Mean | s.d. | min | max |
| 14-3-3 zeta | 0.702 | 0.079 | 0.610 | 0.827 |
| Alpha-tubulin | 0.450 | 0.123 | 0.239 | 0.533 |
| Beta-actin | 0.639 | 0.046 | 0.571 | 0.681 |
| Cathepsin L, sequence 2 | 0.509 | 0.221 | 0.127 | 0.644 |
| c-myc | 0.672 | 0.062 | 0.570 | 0.722 |
| Cytochrome P450 11A1 | 0.677 | 0.180 | 0.364 | 0.810 |
| Gadd153 | 0.502 | 0.096 | 0.354 | 0.589 |
| IgE binding protein | 0.721 | 0.012 | 0.709 | 0.740 |
| L-gulono-gamma-lactone oxidase | 0.680 | 0.277 | 0.329 | 0.886 |
| Matrin F/G | 0.695 | 0.132 | 0.493 | 0.797 |
| MHC class I antigen RT1.A1(f) alpha-chain | 0.475 | 0.139 | 0.360 | 0.707 |
| Nucleoside diphosphate kinase beta isoform | 0.573 | 0.062 | 0.506 | 0.653 |
| Ornithine decarboxylase | 0.666 | 0.068 | 0.608 | 0.764 |
| PAR interacting protein | 0.720 | 0.077 | 0.589 | 0.778 |
| Phase-1 RCT-181 | 0.731 | 0.211 | 0.452 | 0.886 |
| Phase-1 RCT-185 | 0.615 | 0.324 | 0.055 | 0.883 |
| Phase-1 RCT-205 | 0.585 | 0.087 | 0.514 | 0.733 |
| Phase-1 RCT-213 | 0.595 | 0.066 | 0.533 | 0.701 |
| Phase-1 RCT-233 | 0.657 | 0.267 | 0.200 | 0.883 |
| Phase-1 RCT-258 | 0.720 | 0.070 | 0.627 | 0.797 |
| Phase-1 RCT-288 | 0.859 | 0.017 | 0.836 | 0.883 |
| Phase-1 RCT-33 | 0.679 | 0.280 | 0.347 | 0.886 |
| Phase-1 RCT-36 | 0.646 | 0.323 | 0.250 | 0.886 |
| Phase-1 RCT-39 | 0.650 | 0.079 | 0.584 | 0.773 |
| Phase-1 RCT-60 | 0.569 | 0.080 | 0.452 | 0.653 |
| Phase-1 RCT-64 | 0.814 | 0.050 | 0.767 | 0.875 |
| Phase-1 RCT-65 | 0.557 | 0.055 | 0.486 | 0.623 |
| Phase-1 RCT-78 | 0.805 | 0.167 | 0.506 | 0.886 |
| Average Individual Combo 3 | 0.649 | 0.130 | 0.466 | 0.767 |
| Minimum Individual Combo 3 | 0.450 | 0.012 | 0.055 | 0.533 |
| Maximum Individual Combo 3 | 0.859 | 0.324 | 0.836 | 0.886 |

TABLE 14
Comparison of Predictivity for True Liver Inflammation
Classification and Random Classification Using Combo
Gene Sets and Random Subsets and 24 h data

| | | Overall Accuracy** | | | |
|---|---|---|---|---|---|
| Gene | Gene | Correct Classification | | Random Classification | |
| List* | Subset* | Mean | Min-Max | Mean | Min.-Max. |
| Combo | All Genes | 0.860 | (0.785-0.933) | 0.149 | (0.055-0.278) |
| All | | | | | |
| | 5 genes (1) | 0.648 | (0.315-0.886) | 0.479 | (0.178-0.785) |
| | 5 genes (2) | 0.808 | (0.764-0.836) | 0.177 | (0.093-0.278) |
| | 10 genes (1) | 0.839 | (0.759-0.893) | 0.173 | (0.152-0.205) |
| | 10 genes (2) | 0.843 | (0.785-0.909) | 0.199 | (0.107-0.266) |
| | 15 genes (1) | 0.735 | (0.658-0.795) | 0.232 | (0.151-0.292) |
| | 15 genes (2) | 0.799 | (0.696-0.867) | 0.181 | (0.137-0.293) |
| Combo | All Genes | 0.852 | (0.797-0.907) | 0.223 | (0.139-0.354) |
| 5 3 2 | | | | | |
| | 5 genes (1) | 0.766 | (0.722-0.800) | 0.239 | (0.167-0.299) |
| | 5 genes (2) | 0.789 | (0.764-0.818) | 0.177 | (0.133-0.278) |
| | 10 genes (1) | 0.778 | (0.722-0.818) | 0.185 | (0.111-0.234) |
| | 10 genes (2) | 0.813 | (0.764-0.844) | 0.256 | (0.139-0.351) |
| | 15 genes (1) | 0.763 | (0.722-0.840) | 0.205 | (0.111-0.299) |
| | 15 genes (2) | 0.867 | (0.823-0.903) | 0.193 | (0.123-0.253) |
| All- | 5 genes (1) | 0.559 | (0.467-0.625) | 0.244 | (0.187-0.342) |
| Pred | | | | | |
| | 5 genes (2) | 0.612 | (0.519-0.747) | 0.205 | (0.139-0.280) |
| | 10 genes (1) | 0.691 | (0.639-0.787) | 0.219 | (0.152-0.307) |
| | 10 genes (2) | 0.528 | (0.431-0.693) | 0.197 | (0.093-0.293) |
| | 15 genes (1) | 0.509 | (0.456-0.587) | 0.194 | (0.080-0.301) |
| | 15 genes (2) | 0.623 | (0.544-0.733) | 0.220 | (0.167-0.247) |

TABLE 15
Distribution of Compounds* in Individual Training and
Test Sets for 6 Hour Liver Inflammation Data
Training and Test Set 1

| | | Training Set 1 | | | Test Set 1 |
|---|---|---|---|---|---|
| | Training | Positive**- | | | Positive**- |
| Training | Set 1 | Necrosis | | Test Set 1 | Necrosis |
| Set 1 | Positive**- | with | Test Set 1 | Positive**- | with |
| Negative** | Necrosis | Inflammation | Negative** | Necrosis | Inflammation |
| CHLOR-Low⁺ | TET-High⁺ | DMN-High⁺ | HYD-High⁺ | APAP-High⁺ | BRB-Low⁺ |
| TAM-High | CCL4-Low | ANIT-High | CYCA-Low | | CAD-4 |
| BEN-Low | | CCL4-High | GEN-Low | | BRB-High |
| CHEX-High | | LPS-High | ERY-Low | | |
| 5-FU-Low | | AFLB | CMC-Low | | |
| NAL-High | | | PHEN-High | | |
| TAM-Low | | | DOX-Low | | |
| ERY-High | | | ANIT-Low | | |
| PEG-Low | | | QUIN-Low | | |
| HYD-Low | | | 5-FU-Hi | | |
| CPHOS-Low | | | DOX-High | | |
| CAD-Low | | | BAP-High | | |
| CLO-Low | | | CIS-Low | | |
| STRZ-Low | | | KETO-High | | |
| GEN-High | | | CIS-High | | |
| GAN-Low | | | CAR-Low | | |
| CPHOS-High | | | BEN-High | | |
| QUIN-High | | | CLOZ-Low | | |
| NAL-Low | | | CLOZ-High | | |
| EST-Low | | | PBARB-High | | |
| STRZ-High | | | DIF-Low | | |
| THEO-High | | | PHEN-Low | | |
| EST-High | | | KETO-Low | | |
| ETH-Low | | | AMPB-Low | | |
| PBARB-Low | | | GAN-High | | |
| CAR-High | | | | | |
| TET-Low | | | | | |
| CHCL3-Low | | | | | |
| AMPB-Hi | | | | | |
| CHCL3-High | | | | | |
| ISON-Low | | | | | |
| THEO-Low | | | | | |
| MET-High | | | | | |
| PUR-High | | | | | |
| CLO-High | | | | | |
| DEX-High | | | | | |
| APAP-Low | | | | | |
| BUS-Low | | | | | |
| PUR-Low | | | | | |
| DIF-High | | | | | |
| CAD-High | | | | | |
| BAP-Low | | | | | |
| LPS-Low | | | | | |
| ISON-High | | | | | |
| CHLOR-High | | | | | |
| MET-Low | | | | | |
| CHEX-Low | | | | | |
| DEX-Low | | | | | |
| BUS-High | | | | | |
| CYCA-High | | | | | |

Training and Test Set 2

| | Training | Training Set 2 | | | Test Set 2 |
|---|---|---|---|---|---|
| Training | Set 2 | Positive- | | Test Set 2 | Positive- |
| Set 2 | Positive- | Necrosis with | Test Set 2 | Positive- | Necrosis with |
| Negative | Necrosis | Inflammation | Negative | Necrosis | Inflammation |
| QUIN-High | CCL4-Low | LPS-High | QUIN-Low | TET-High | DMN-High |
| DOX-Low | APAP-High | AFLB | CMC-Low | | BRB-Low |
| CHEX-Low | | BRB-High | CLO-High | | CAD-4 |
| THEO-Low | | ANIT-High | STRZ-Low | | |
| BUS-Low | | CCL4-High | BUS-High | | |
| STRZ-High | | | ISON-High | | |
| CPHOS-Low | | | CYCA-High | | |
| GAN-High | | | THEO-High | | |
| BEN-Low | | | CLO-Low | | |
| EST-High | | | AMPB-Hi | | |
| ANIT-Low | | | CYCA-Low | | |
| HYD-High | | | CHCL3-High | | |
| DIF-Low | | | CLOZ-Low | | |
| ISON-Low | | | GEN-Low | | |
| GAN-Low | | | AMPB-Low | | |
| KETO-High | | | TET-Low | | |
| PBARB-Low | | | CAD-Low | | |
| PHEN-High | | | NAL-Low | | |
| BEN-High | | | CHLOR-Low | | |
| CIS-Low | | | ERY-High | | |
| CHLOR-High | | | GEN-High | | |
| ETH-Low | | | PUR-High | | |
| CLOZ-High | | | DIF-High | | |
| PUR-Low | | | HYD-Low | | |
| CHCL3-Low | | | DOX-High | | |
| PHEN-Low | | | | | |
| ERY-Low | | | | | |
| 5-FU-Hi | | | | | |
| CAR-High | | | | | |
| MET-High | | | | | |
| CIS-High | | | | | |
| 5-FU-Low | | | | | |
| CHEX-High | | | | | |
| TAM-High | | | | | |
| EST-Low | | | | | |
| APAP-Low | | | | | |
| NAL-High | | | | | |
| LPS-Low | | | | | |
| CPHOS-High | | | | | |
| CAD-High | | | | | |
| MET-Low | | | | | |
| BAP-High | | | | | |
| TAM-Low | | | | | |
| KETO-Low | | | | | |
| BAP-Low | | | | | |
| DEX-Low | | | | | |
| PBARB-High | | | | | |
| DEX-High | | | | | |
| CAR-Low | | | | | |
| PEG-Low | | | | | |

Training and Test Set 3

| | Training | Training Set 3 | | | Test Set 3 |
|---|---|---|---|---|---|
| Training | Set 3 | Positive- | | Test Set 3 | Positive- |
| Set 3 | Positive- | Necrosis with | Test Set 3 | Positive- | Necrosis with |
| Negative | Necrosis | Inflammation | Negative | Necrosis | Inflammation |
| CPHOS-Low | TET-High | ANIT-High | ISON-Low | CCL4-Low | CAD-4 |
| CHEX-High | APAP-High | BRB-Low | QUIN-High | | BRB-High |
| THEO-Low | | AFLB | NAL-High | | LPS-High |
| AMPB-Low | | DMN-High | CHEX-Low | | |
| 5-FU-Low | | CCL4-High | ETH-Low | | |
| CHLOR-High | | | TAM-High | | |
| APAP-Low | | | GAN-Low | | |
| THEO-High | | | BUS-High | | |
| STRZ-High | | | STRZ-Low | | |
| CPHOS-High | | | NAL-Low | | |
| DEX-High | | | PHEN-Low | | |
| ISON-High | | | BAP-High | | |
| HYD-High | | | CLO-High | | |
| BEN-High | | | PHEN-High | | |
| CAR-Low | | | ERY-Low | | |
| 5-FU-Hi | | | PEG-Low | | |
| CLO-Low | | | LPS-Low | | |
| EST-Low | | | CLOZ-High | | |
| CAR-High | | | GAN-High | | |
| CIS-High | | | GEN-Low | | |
| CHCL3-High | | | DIF-Low | | |
| PUR-High | | | PBARB-Low | | |
| BEN-Low | | | KETO-Low | | |
| CLOZ-Low | | | PBARB-High | | |
| BAP-Low | | | PUR-Low | | |
| CHCL3-Low | | | | | |
| TAM-Low | | | | | |
| DIF-High | | | | | |
| DEX-Low | | | | | |
| ANIT-Low | | | | | |
| CYCA-High | | | | | |
| DOX-High | | | | | |
| TET-Low | | | | | |
| GEN-High | | | | | |
| BUS-Low | | | | | |
| CMC-Low | | | | | |
| AMPB-Hi | | | | | |
| MET-High | | | | | |
| HYD-Low | | | | | |
| CIS-Low | | | | | |
| QUIN-Low | | | | | |
| CYCA-Low | | | | | |
| CAD-Low | | | | | |
| MET-Low | | | | | |
| DOX-Low | | | | | |
| KETO-High | | | | | |
| CHLOR-Low | | | | | |
| CAD-High | | | | | |
| ERY-High | | | | | |
| EST-High | | | | | |

Training and Test Set 4

| | Training | Training Set 4 | | | Test Set 4 |
|---|---|---|---|---|---|
| Training | Set 4 | Positive- | | Test Set 4 | Positive- |
| Set 4 | Positive- | Necrosis with | Test Set 4 | Positive- | Necrosis with |
| Negative | Necrosis | Inflammation | Negative | Necrosis | Inflammation |
| ERY-Low | TET-High | CAD-4 | TET-Low | APAP-High | DMN-High |
| BAP-Low | CCL4-Low | AFLB | GEN-High | | BRB-High |
| MET-High | | BRB-Low | KETO-Low | | ANIT-High |
| ISON-High | | LPS-High | DEX-High | | |
| DIF-Low | | CCL4-High | CAR-High | | |
| 5-FU-Hi | | | CLO-Low | | |
| HYD-High | | | CAD-Low | | |
| PUR-High | | | CHLOR-High | | |
| THEO-Low | | | DOX-Low | | |
| DEX-Low | | | 5-FU-Low | | |
| QUIN-Low | | | CHCL3-High | | |
| CHCL3-Low | | | AMPB-Hi | | |
| THEO-High | | | DIF-High | | |
| PEG-Low | | | CPHOS-Low | | |
| EST-Low | | | STRZ-Low | | |
| CHEX-High | | | QUIN-High | | |
| AMPB-Low | | | CHEX-Low | | |
| CYCA-High | | | CLO-High | | |
| LPS-Low | | | BUS-Low | | |
| CLOZ-Low | | | GAN-High | | |
| TAM-Low | | | ISON-Low | | |
| GEN-Low | | | TAM-High | | |
| BAP-High | | | BUS-High | | |
| CIS-Low | | | DOX-High | | |
| BEN-Low | | | CMC-Low | | |
| KETO-High | | | | | |
| CPHOS-High | | | | | |
| STRZ-High | | | | | |
| CIS-High | | | | | |
| HYD-Low | | | | | |
| NAL-Low | | | | | |
| MET-Low | | | | | |
| PHEN-High | | | | | |
| ETH-Low | | | | | |
| CHLOR-Low | | | | | |
| CLOZ-High | | | | | |
| PBARB-Low | | | | | |
| BEN-High | | | | | |
| APAP-Low | | | | | |
| ERY-High | | | | | |
| EST-High | | | | | |
| PUR-Low | | | | | |
| CYCA-Low | | | | | |
| CAR-Low | | | | | |
| ANIT-Low | | | | | |
| GAN-Low | | | | | |
| PBARB-High | | | | | |
| NAL-High | | | | | |
| PHEN-Low | | | | | |
| CAD-High | | | | | |

Training and Test Set 5

| | Training | Training Set 5 | | | Test Set 5 |
|---|---|---|---|---|---|
| Training | Set 5 | Positive- | | Test Set 5 | Positive- |
| Set 5 | Positive- | Necrosis with | Test Set 5 | Positive- | Necrosis with |
| Negative | Necrosis | Inflammation | Negative | Necrosis | Inflammation |
| CAR-Low | APAP-High | BRB-High | BUS-High | TET-High | CCL4-High |
| TET-Low | CCL4-Low | LPS-High | ISON-High | | BRB-Low |
| QUIN-Low | | DMN-High | CMC-Low | | AFLB |
| CPHOS-Low | | ANIT-High | AMPB-Low | | |
| MET-High | | CAD-4 | HYD-Low | | |
| 5-FU-Hi | | | GEN-High | | |
| GAN-Low | | | BAP-High | | |
| DOX-High | | | PBARB-High | | |
| BAP- Low | | | CIS-High | | |
| BEN-Low | | | PHEN-High | | |
| CHEX-High | | | ERY-High | | |
| NAL-High | | | KETO-High | | |
| PBARB-Low | | | THEO-High | | |
| STRZ-High | | | BUS-Low | | |
| PEG-Low | | | CHCL3-Low | | |
| ERY-Low | | | EST-High | | |
| DIF-Low | | | APAP-Low | | |
| AMPB-Hi | | | CHLOR-High | | |
| PUR-High | | | CAD-High | | |
| GEN-Low | | | 5-FU-Low | | |
| ETH-Low | | | CYCA-High | | |
| GAN-High | | | ISON-Low | | |
| CYCA-Low | | | PHEN-Low | | |
| CLOZ-High | | | MET-Low | | |
| HYD-High | | | PUR-Low | | |
| NAL-Low | | | | | |
| CHLOR-Low | | | | | |
| CLO-Low | | | | | |
| CAR-High | | | | | |
| TAM-Low | | | | | |
| STRZ-Low | | | | | |
| CPHOS-High | | | | | |
| CLO-High | | | | | |
| CHEX-Low | | | | | |
| THEO-Low | | | | | |
| ANIT-Low | | | | | |
| DOX-Low | | | | | |
| CIS-Low | | | | | |
| DEX-High | | | | | |
| TAM-High | | | | | |
| EST-Low | | | | | |
| DIF-High | | | | | |
| DEX-Low | | | | | |
| CLOZ-Low | | | | | |
| CHCL3-High | | | | | |
| KETO-Low | | | | | |
| CAD-Low | | | | | |
| QUIN-High | | | | | |
| LPS-Low | | | | | |
| BEN-High | | | | | |

TABLE 16
List of Genes, Whose Expression at 6 h Directly Correlates
with Liver Inflammation at 72 h, Ranked by Pearson
Correlation Coefficient

| | Correlation |
|---|---|
| Gene | Coefficient |
| Phase-1 RCT-207 | 0.383 |
| Phase-1 RCT-59 | 0.356 |
| c-jun | 0.346 |
| Phase-1 RCT-50 | 0.327 |
| Cyclin G | 0.321 |
| Phase-1 RCT-144 | 0.320 |
| Gadd153 | 0.317 |
| ID-1 | 0.313 |
| Heme oxygenase | 0.310 |
| Zinc finger protein | 0.300 |
| NIPK | 0.299 |
| Phase-1 RCT-179 | 0.295 |
| Phase-1 RCT-197 | 0.293 |
| Gadd45 | 0.293 |
| Activating transcription factor 3 | 0.275 |
| c-myc | 0.274 |
| Melanoma-associated antigen ME491 | 0.270 |
| Beta-tubulin, class I | 0.265 |
| Phase-1 RCT-49 | 0.260 |
| Waf1 | 0.259 |
| 14-3-3 zeta | 0.253 |
| Phase-1 RCT-225 | 0.252 |
| Cathepsin L, sequence 2 | 0.248 |
| Phase-1 RCT-212 | 0.247 |
| Phase-1 RCT-242 | 0.243 |
| Ferritin H-chain | 0.235 |
| Phase-1 RCT-62 | 0.232 |
| Phase-1 RCT-75 | 0.232 |
| Argininosuccinate lyase | 0.230 |
| Phase-1 RCT-156 | 0.230 |
| Caspase 6 | 0.229 |
| Insulin-like growth factor binding protein 1 | 0.227 |
| Phase-1 RCT-228 | 0.227 |
| Phase-1 RCT-109 | 0.225 |
| Integrin beta1 | 0.224 |
| Colony-stimulating factor-1 | 0.223 |
| Phase-1 RCT-111 | 0.221 |
| Phase-1 RCT-191 | 0.220 |
| Phase-1 RCT-72 | 0.220 |
| Phase-1 RCT-103 | 0.220 |
| Phase-1 RCT-12 | 0.218 |
| Matrix metalloproteinase-1 | 0.217 |
| Phase-1 RCT-127 | 0.216 |
| NGF-inducible anti-proliferative putative secreted | 0.216 |
| protein (PC3) | |
| Phase-1 RCT-171 | 0.215 |
| Macrophage inflammatory protein-1 alpha | 0.212 |
| Phase-1 RCT-259 | 0.211 |
| MHC class I antigen RT1.A1(f) alpha-chain | 0.210 |
| Phase-1 RCT-95 | 0.208 |
| Phase-1 RCT-235 | 0.204 |
| Phase-1 RCT-55 | 0.203 |
| Phase-1 RCT-221 | 0.202 |
| Ubiquitin conjugating enzyme (RAD 6 homologue) | 0.202 |
| Macrophage inflammatory protein-2 alpha | 0.201 |

TABLE 17
List of Genes, Whose Expression at 6 h Inversely Correlates
with Liver Inflammation at 72 h, Ranked by Spearman
Correlation Coefficient

| | Correlation |
|---|---|
| Gene | Coefficient |
| Diacylglycerol kinase zeta | −0.150 |
| Carbamyl phosphate synthetase I | −0.151 |
| Phase-1 RCT-28 | −0.152 |
| Cyclin D3 | −0.154 |
| 3-methyladenine DNA glycosylase | −0.154 |
| Phase-1 RCT-63 | −0.155 |
| 8-oxoguanine DNA glycosylase | −0.156 |
| Cholesterol 7-alpha-hydroxylase (P450 VII) | −0.160 |
| Phase-1 RCT-141 | −0.160 |
| Peroxisome assembly factor 1 | −0.161 |
| Phase-1 RCT-184 | −0.161 |
| Phase-1 RCT-260 | −0.162 |
| Glutamine synthetase | −0.162 |
| Vesicular monoamine transporter (VMAT) | −0.162 |
| Phase-1 RCT-112 | −0.167 |
| Inositol polyphosphate multikinase (Ipmk) | −0.168 |
| Phase-1 RCT-280 | −0.171 |
| Matrin F/G | −0.172 |
| Selenoprotein P | −0.172 |
| Complement component C3 | −0.172 |
| Phase-1 RCT-32 | −0.172 |
| Phase-1 RCT-13 | −0.174 |
| Phase-1 RCT-114 | −0.175 |
| Organic anion transporter K1 | −0.176 |
| Phase-1 RCT-82 | −0.176 |
| Phase-1 RCT-168 | −0.177 |
| Carbonic anhydrase II | −0.179 |
| Cytochrome P450 2E1 | −0.181 |
| Stem cell factor | −0.183 |
| Phase-1 RCT-83 | −0.184 |
| C4b-binding protein | −0.184 |
| Phase-1 RCT-140 | −0.185 |
| JNK1 stress activated protein kinase | −0.187 |
| Peroxisomal multifunctional enzyme type II | −0.189 |
| Cyclin dependent kinase 4 | −0.189 |
| Organic anion transporter 3 | −0.190 |
| Alcohol dehydrogenase 1 | −0.190 |
| Phase-1 RCT-139 | −0.196 |
| Emerin | −0.199 |
| Phase-1 RCT-173 | −0.205 |
| Nucleosome assembly protein | −0.207 |
| Phase-1 RCT-73 | −0.209 |
| Phase-1 RCT-214 | −0.214 |
| Phase-1 RCT-119 | −0.215 |
| Tryptophan hydroxylase | −0.216 |
| PTEN/MMAC1 | −0.217 |
| Thymidylate synthase | −0.220 |
| DNA topoisomerase I | −0.223 |
| Phase-1 RCT-40 | −0.228 |
| Sarcoplasmic reticulum calcium ATPase | −0.228 |
| Protein tyrosine phosphatase alpha | −0.238 |
| Carbonic anhydrase III | −0.243 |
| 3-beta-hydroxysteroid dehydrogenase (HSD3B1) | −0.256 |
| Phase-1 RCT-161 | −0.261 |
| Glucokinase | −0.265 |
| Senescence marker protein-30 | −0.275 |
| Acetyl-CoA carboxylase | −0.294 |

TABLE 18
List of genes whose expression at 6 hours is
predictive of liver inflammation at 72 hours

| | Combination* (No. |
|---|---|
| Gene | of Occurrences) |
| Gadd153 | 5 |
| Argininosuccinate lyase | 4 |
| Beta-tubulin, class I | 4 |
| Cathepsin L, sequence 2 | 4 |
| c-myc | 4 |
| Heme oxygenase | 4 |
| Insulin-like growth factor binding protein 1 | 4 |
| Integrin beta1 | 4 |
| Interferon related developmental regulator IFRD1 | 4 |
| (PC4) | |
| Monoamine oxidase B | 4 |
| NIPK | 4 |
| Phase-1 RCT-127 | 4 |
| Phase-1 RCT-197 | 4 |
| Phase-1 RCT-207 | 4 |
| Phase-1 RCT-242 | 4 |
| Phase-1 RCT-50 | 4 |
| Phase-1 RCT-72 | 4 |
| Phase-1 RCT-75 | 4 |
| Senescence marker protein-30 | 4 |
| 8-oxoguanine DNA glycosylase | 3 |
| Axin | 3 |
| C4b-binding protein | 3 |
| Carbamyl phosphate synthetase I | 3 |
| Caspase 6 | 3 |
| c-jun | 3 |
| Cyclin G | 3 |
| Gadd45 | 3 |
| ID-1 | 3 |
| JNK1 stress activated protein kinase | 3 |
| Macrophage inflammatory protein-1 alpha | 3 |
| NGF-inducible anti-proliferative putative secreted | 3 |
| protein (PC3) | |
| Peroxisome proliferator activated receptor gamma | 3 |
| Phase-1 RCT-161 | 3 |
| Phase-1 RCT-168 | 3 |
| Phase-1 RCT-184 | 3 |
| Phase-1 RCT-214 | 3 |
| Phase-1 RCT-225 | 3 |
| Phase-1 RCT-287 | 3 |
| Phase-1 RCT-40 | 3 |
| Phase-1 RCT-49 | 3 |
| Phase-1 RCT-89 | 3 |
| Selenoprotein P | 3 |
| Stem cell factor | 3 |
| Zinc finger protein | 3 |
| Phase-1 RCT-171 | 2 |
| 14-3-3 zeta | 2 |
| 3-methyladenine DNA glycosylase | 2 |
| Acetyl-CoA carboxylase | 2 |
| Alcohol dehydrogenase 1 | 2 |
| Alpha-fetoprotein | 2 |
| AT-3 | 2 |
| Carbonic anhydrase III | 2 |
| Cholesterol 7-alpha-hydroxylase (P450 VII) | 2 |
| Ciliary neurotrophic factor | 2 |
| Cofilin | 2 |
| Colony-stimulating factor-1 | 2 |
| Cytochrome P450 2E1 | 2 |
| DNA binding protein inhibitor ID2 | 2 |
| DNA polymerase beta | 2 |
| DNA topoisomerase I | 2 |
| Elongation factor-1 alpha | 2 |
| Emerin | 2 |
| Equilbrative nitrobenzylthioinosine-sensitive | 2 |
| nucleoside transporter | |
| Ferritin H-chain | 2 |
| Fetuin beta (Fetub) | 2 |
| Gamma-actin, cytoplasmic | 2 |
| Glucokinase | 2 |
| Glucose-regulated protein 78 | 2 |
| Glutathione S-transferase theta-1 | 2 |
| HMG CoA reductase | 2 |
| Insulin-like growth factor I | 2 |
| Iron-responsive element-binding protein | 2 |
| Matrin F/G | 2 |
| Melanoma-associated antigen ME491 | 2 |
| Multidrug resistant protein-2 | 2 |
| NADP-dependent isocitrate dehydrogenase, | 2 |
| cytosolic | |
| Nucleosome assembly protein | 2 |
| Peroxisomal multifunctional enzyme type II | 2 |
| Peroxisome assembly factor 1 | 2 |
| Phase-1 RCT-252 | 2 |
| Phase-1 RCT-109 | 2 |
| Protein O-mannosyltransferase 1 (Pomt1) | 2 |
| Phase-1 RCT-123 | 2 |
| Phase-1 RCT-141 | 2 |
| Phase-1 RCT-144 | 2 |
| Phase-1 RCT-166 | 2 |
| Phase-1 RCT-169 | 2 |
| Phase-1 RCT-173 | 2 |
| Phase-1 RCT-179 | 2 |
| Phase-1 RCT-18 | 2 |
| Phase-1 RCT-191 | 2 |
| Phase-1 RCT-221 | 2 |
| Phase-1 RCT-251 | 2 |
| Phase-1 RCT-270 | 2 |
| Phase-1 RCT-28 | 2 |
| Phase-1 RCT-289 | 2 |
| Phase-1 RCT-297 | 2 |
| Phase-1 RCT-32 | 2 |
| Phase-1 RCT-55 | 2 |
| Phase-1 RCT-59 | 2 |
| Phase-1 RCT-62 | 2 |
| Phase-1 RCT-63 | 2 |
| Phase-1 RCT-65 | 2 |
| Phase-1 RCT-66 | 2 |
| Phase-1 RCT-71 | 2 |
| Phase-1 RCT-73 | 2 |
| Phase-1 RCT-82 | 2 |
| Phase-1 RCT-9 | 2 |
| Phase-1 RCT-95 | 2 |
| Proliferating cell nuclear antigen gene | 2 |
| Pyruvate kinase, muscle | 2 |
| Ribosomal protein L13A | 2 |
| Thioredoxin-1 (Trx1) | 2 |
| Thymidylate synthase | 2 |
| Cyclin-dependent kinase 4 inhibitor P27kip1 | 1 |
| (alternate clone) | |
| Cytochrome P450 2C39 (alternate clone 2) | 1 |
| 3-beta-hydroxysteroid dehydrogenase (HSD3B1) | 1 |
| 3-hydroxyisobutyrate dehydrogenase | 1 |
| Activating transcription factor 3 | 1 |
| Activin receptor type II | 1 |
| Acyl-CoA dehydrogenase, medium chain | 1 |
| Adenine nucleotide translocator 1 | 1 |
| Alpha-1 acid glycoprotein | 1 |
| Alpha-1 microglobulin/bikunin precursor (Ambp) | 1 |
| Alpha-2-macroglobulin, sequence 2 | 1 |
| Alpha-2-microglobulin | 1 |
| Apolipoprotein E | 1 |
| Aryl sulfotransferase | 1 |
| Urinary protein 2 precursor | 1 |
| Carbonic anhydrase II | 1 |
| Carbonic anhydrase III, sequence 2 | 1 |
| Carbonyl reductase | 1 |
| Ceruloplasmin | 1 |
| Complement component C3 | 1 |
| Complement factor I (CFI) | 1 |
| Cyclin D3 | 1 |
| Cystatin C | 1 |
| Cytochrome P450 1A2 | 1 |
| Cytochrome P450 2C11 | 1 |
| Diacylglycerol kinase zeta | 1 |
| Disulfide isomerase related protein (ERp72) | 1 |
| Dynamin-1 (D100) | 1 |
| Endogenous retroviral sequence, 5′ and 3′ LTR | 1 |
| Epoxide hydrolase | 1 |
| Focal adhesion kinase (pp125FAK) | 1 |
| Gap junction membrane channel protein beta 1 | 1 |
| (Gjb1) | |
| Glucose transporter 2 | 1 |
| Glutamine synthetase | 1 |
| Glutathione S-transferase Yb2 subunit | 1 |
| Glutathione S-transferase P1 | 1 |
| Glutathione S-transferase Ya | 1 |
| Glycine methyltransferase | 1 |
| Hepatic lipase | 1 |
| Hypoxia-inducible factor 1 alpha | 1 |
| IkB-a | 1 |
| Insulin-like growth factor binding protein 5 | 1 |
| Integrin beta-4 | 1 |
| Inter-alpha-inhibitor H4 heavy chain (Itih4) | 1 |
| Liver fatty acid binding protein | 1 |
| Lysyl oxidase | 1 |
| Macrophage inflammatory protein-2 alpha | 1 |
| Malate dehydrogenase, cytosolic | 1 |
| Matrix metalloproteinase-1 | 1 |
| Methylacyl-CoA racemase alpha | 1 |
| MHC class I antigen RT1.A1(f) alpha-chain | 1 |
| MHC class II antigen RT1.B-1 beta-chain | 1 |
| Multidrug resistant protein-1 | 1 |
| NADPH cytochrome P450 oxidoreductase | 1 |
| N-cadherin | 1 |
| Organic anion transporter 3 | 1 |
| Organic anion transporting polypeptide 1 | 1 |
| Organic cation transporter 3 | 1 |
| Osteopontin | 1 |
| Phase-1 RCT-10 | 1 |
| Phase-1 RCT-103 | 1 |
| Phase-1 RCT-108 | 1 |
| Phase-1 RCT-111 | 1 |
| Phase-1 RCT-112 | 1 |
| Phase-1 RCT-113 | 1 |
| Phase-1 RCT-114 | 1 |
| Phase-1 RCT-117 | 1 |
| Phase-1 RCT-119 | 1 |
| Phase-1 RCT-12 | 1 |
| Phase-1 RCT-13 | 1 |
| Phase-1 RCT-136 | 1 |
| Phase-1 RCT-137 | 1 |
| Phase-1 RCT-138 | 1 |
| Phase-1 RCT-140 | 1 |
| Phase-1 RCT-142 | 1 |
| Phase-1 RCT-143 | 1 |
| Phase-1 RCT-145 | 1 |
| Phase-1 RCT-148 | 1 |
| Phase-1 RCT-15 | 1 |
| Phase-1 RCT-151 | 1 |
| Phase-1 RCT-156 | 1 |
| Phase-1 RCT-158 | 1 |
| Phase-1 RCT-164 | 1 |
| Phase-1 RCT-180 | 1 |
| Phase-1 RCT-189 | 1 |
| Phase-1 RCT-192 | 1 |
| Phase-1 RCT-195 | 1 |
| Phase-1 RCT-202 | 1 |
| Phase-1 RCT-204 | 1 |
| Calgranulin B | 1 |
| Phase-1 RCT-212 | 1 |
| Phase-1 RCT-22 | 1 |
| Phase-1 RCT-235 | 1 |
| Phase-1 RCT-240 | 1 |
| Phase-1 RCT-241 | 1 |
| Phase-1 RCT-25 | 1 |
| Phase-1 RCT-258 | 1 |
| Phase-1 RCT-259 | 1 |
| Phase-1 RCT-260 | 1 |
| Phase-1 RCT-261 | 1 |
| Phase-1 RCT-264 | 1 |
| Phase-1 RCT-278 | 1 |
| Phase-1 RCT-280 | 1 |
| Phase-1 RCT-281 | 1 |
| Phase-1 RCT-288 | 1 |
| Phase-1 RCT-29 | 1 |
| Phase-1 RCT-290 | 1 |
| Phase-1 RCT-294 | 1 |
| Phase-1 RCT-3 | 1 |
| Phase-1 RCT-34 | 1 |
| Phase-1 RCT-39 | 1 |
| Phase-1 RCT-42 | 1 |
| Phase-1 RCT-43 | 1 |
| Phase-1 RCT-45 | 1 |
| Phase-1 RCT-53 | 1 |
| Phase-1 RCT-54 | 1 |
| Phase-1 RCT-56 | 1 |
| Phase-1 RCT-76 | 1 |
| Phase-1 RCT-83 | 1 |
| Phase-1 RCT-90 | 1 |
| Phase-1 RCT-91 | 1 |
| Phase-1 RCT-96 | 1 |
| Phosphatidylethanolamine-binding protein | 1 |
| Phospholipase D | 1 |
| Prostaglandin H synthase | 1 |
| Protein tyrosine phosphatase alpha | 1 |
| PTEN/MMAC1 | 1 |
| Retinol-binding protein (RBP) | 1 |
| Ribosomal protein L13 | 1 |
| Ribosomal protein S9 | 1 |
| Sarcoplasmic reticulum calcium ATPase | 1 |
| Stathmin | 1 |
| Superoxide dismutase Mn | 1 |
| Syndecan-1 | 1 |
| Tissue factor pathway inhibitor | 1 |
| Tissue plasminogen activator | 1 |
| Tryptophan hydroxylase | 1 |
| Ubiquitin conjugating enzyme (RAD 6 homologue) | 1 |
| UDP-glucuronosyltransferase | 1 |
| Vascular endothelial growth factor | 1 |
| Very long-chain acyl-CoA synthetase | 1 |
| Vesicular monoamine transporter (VMAT) | 1 |
| VL30 element | 1 |
| Waf1 | 1 |

TABLE 19
Comparison of Predictivity for True Liver Inflammation
Classification and Random Classification Using
Combo Gene Sets and 6 h data

| | Overall Accuracy** | | | |
|---|---|---|---|---|
| | Correct Classification | | Random Classification | |
| Gene List* | Mean | Min-Max | Mean | Min.-Max. |
| Combo All | 0.736 | (0.638-0.815) | 0.405 | (0.321-0.463) |
| Combo 5 | 0.660 | (0.364-0.788) | 0.448 | (0.210-0.597) |
| Combo 4 | 0.767 | (0.650-0.840) | 0.302 | (0.150-0.378) |
| Combo 3 | 0.745 | (0.700-0.802) | 0.357 | (0.309-0.425) |
| Combo 2 | 0.698 | (0.538-0.770) | 0.361 | (0.325-0.420) |
| Combo 1 | 0.515 | (0.338-0.679) | 0.378 | (0.257-0.455) |

TABLE 20
Distribution of Compounds* in Individual Training and
Test Sets for 72 Hour Liver Inflammation Data
Training and Test Set 1

| | Training | Training Set 1 | | | Test Set 1 |
|---|---|---|---|---|---|
| Training | Set 1 | Positive**- | | Test Set 1 | Positive**- |
| Set 1 | Positive**- | Necrosis with | Test Set 1 | Positive**- | Necrosis with |
| Negative** | Necrosis | Inflammation | Negative** | Necrosis | Inflammation |
| 5-FU-High⁺ | CCL4-Low⁺ | CCL4-High⁺ | 5-FU-Low⁺ | APAP-High⁺ | ANIT-High⁺ |
| AMPB-Low | TET-High | BRB-High | THEO-Low | | DMN |
| APAP-Low | | AFLB | AMPB-High | | |
| AZA-High | | BRB-Low | ANIT-Low | | |
| AZA-Low | | LPS-High | CAD-Low | | |
| BAP | | | CHCL3-High | | |
| BEN-High | | | CHEX-High | | |
| BEN-Low | | | CHEX-Low | | |
| BUS | | | CLOZ-High | | |
| CAD-High | | | CLOZ-Low | | |
| CAR | | | CYCA-High | | |
| CHCL3-Low | | | DEX-Low | | |
| CHLOR-High | | | ERY-High | | |
| CHLOR-Low | | | GAN-Low | | |
| CIS-High | | | GEN-Low | | |
| CIS-Low | | | HYD-Low | | |
| CLO-High | | | PHEN-High | | |
| CLO-Low | | | PUR-High | | |
| CMC | | | PUR-Low | | |
| CPHOS-High | | | QUIN-High | | |
| CPHOS-Low | | | TET-Low | | |
| CYCA-Low | | | THEO-High | | |
| DEX-High | | | | | |
| DIF-High | | | | | |
| DIF- Low | | | | | |
| DOX | | | | | |
| ERY-Low | | | | | |
| EST-High | | | | | |
| EST-Low | | | | | |
| ETH | | | | | |
| GAN-High | | | | | |
| GEN-High | | | | | |
| HYD-High | | | | | |
| ISON-High | | | | | |
| ISON-Low | | | | | |
| KETO-High | | | | | |
| KETO-Low | | | | | |
| LPS-Low | | | | | |
| MET | | | | | |
| NAL-High | | | | | |
| NAL-Low | | | | | |
| PBARB-High | | | | | |
| PBARB-Low | | | | | |
| PEG | | | | | |
| PHEN-Low | | | | | |
| QUIN-Low | | | | | |
| STRZ-High | | | | | |
| STRZ-Low | | | | | |
| TAM-High | | | | | |
| TAM-Low | | | | | |

Training and Test Set 2

| | Training | Training Set 2 | | | Test Set 2 |
|---|---|---|---|---|---|
| Training | Set 2 | Positive- | | Test Set 2 | Positive- |
| Set 2 | Positive- | Necrosis with | Test Set 2 | Positive- | Necrosis with |
| Negative | Necrosis | Inflammation | Negative | Necrosis | Inflammation |
| PEG | CCL4-Low | AFLB | ANIT-Low | APAP-High | DMN |
| 5-FU-High | TET-High | ANIT-High | APAP-Low | | BRB-Low |
| 5-FU-Low | | BRB-High | BAP | | |
| AMPB-High | | CCL4-High | BEN-High | | |
| AMPB-Low | | LPS-High | CHEX-Low | | |
| AZA-High | | | CIS-High | | |
| AZA-Low | | | CLO-Low | | |
| BEN-Low | | | CMC | | |
| BUS | | | CPHOS-Low | | |
| CAD-High | | | CYCA-High | | |
| CAD-Low | | | DEX-Low | | |
| CAR | | | EST-Low | | |
| CHCL3-High | | | GEN-Low | | |
| CHCL3-Low | | | ISON-Low | | |
| CHEX-High | | | LPS-Low | | |
| CHLOR-High | | | NAL-High | | |
| CHLOR-Low | | | PBARB-High | | |
| CIS-Low | | | PUR-Low | | |
| CLO-High | | | QUIN-High | | |
| CLOZ-High | | | STRZ-High | | |
| CLOZ-Low | | | STRZ-Low | | |
| CPHOS-High | | | THEO-Low | | |
| CYCA-Low | | | | | |
| DEX-High | | | | | |
| DIF-High | | | | | |
| DIF-Low | | | | | |
| DOX | | | | | |
| ERY-High | | | | | |
| ERY-Low | | | | | |
| EST-High | | | | | |
| ETH | | | | | |
| GAN-High | | | | | |
| GAN-Low | | | | | |
| GEN-High | | | | | |
| HYD-High | | | | | |
| HYD-Low | | | | | |
| ISON-High | | | | | |
| KETO-High | | | | | |
| KETO-Low | | | | | |
| MET | | | | | |
| NAL-Low | | | | | |
| PBARB-Low | | | | | |
| PHEN-High | | | | | |
| PHEN-Low | | | | | |
| PUR-High | | | | | |
| QUIN-Low | | | | | |
| TAM-High | | | | | |
| TAM-Low | | | | | |
| TET-Low | | | | | |
| THEO-High | | | | | |

Training and Test Set 3

| | Training | Training Set 3 | | | Test Set 3 |
|---|---|---|---|---|---|
| Training | Set 3 | Positive- | | Test Set 3 | Positive- |
| Set 3 | Positive- | Necrosis with | Test Set 3 | Positive- | Necrosis with |
| Negative | Necrosis | Inflammation | Negative | Necrosis | Inflammation |
| 5-FU-High | APAP-High | AFLB | AMPB-Low | TET-High | LPS-High |
| 5-FU-Low | CCL4-LOW | ANIT-High | ANIT-Low | | CCL4-High |
| AMPB-High | | BRB-High | AZA-Low | | |
| APAP-Low | | BRB-Low | BEN-Low | | |
| AZA-High | | DMN | CHCL3-LOW | | |
| BAP | | | CHEX-High | | |
| BEN-High | | | CIS-Low | | |
| BUS | | | CLO-High | | |
| CAD-High | | | CLO-Low | | |
| CAD-Low | | | CYCA-Low | | |
| CAR | | | DIF-High | | |
| CHCL3-High | | | ERY-Low | | |
| CHEX-Low | | | EST-Low | | |
| CHLOR-High | | | GAN-High | | |
| CHLOR-Low | | | GAN-Low | | |
| CIS-High | | | HYD-Low | | |
| CLOZ-High | | | ISON-Low | | |
| CLOZ-Low | | | LPS-Low | | |
| CMC | | | NAL-Low | | |
| CPHOS-High | | | PUR-Low | | |
| CPHOS-Low | | | STRZ-High | | |
| CYCA-High | | | STRZ-Low | | |
| DEX-High | | | | | |
| DEX-Low | | | | | |
| DIF-Low | | | | | |
| DOX | | | | | |
| ERY-High | | | | | |
| EST-High | | | | | |
| ETH | | | | | |
| GEN-High | | | | | |
| GEN-Low | | | | | |
| HYD-High | | | | | |
| ISON-High | | | | | |
| KETO-High | | | | | |
| KETO-Low | | | | | |
| MET | | | | | |
| NAL-High | | | | | |
| PBARB-High | | | | | |
| PBARB-Low | | | | | |
| PEG | | | | | |
| PHEN-High | | | | | |
| PHEN-Low | | | | | |
| PUR-High | | | | | |
| QUIN-High | | | | | |
| QUIN-Low | | | | | |
| TAM-High | | | | | |
| TAM-Low | | | | | |
| TET-Low | | | | | |
| THEO-High | | | | | |
| THEO-Low | | | | | |

Training and Test Set 4

| | Training | Training Set 4 | | | Test Set 4 |
|---|---|---|---|---|---|
| Training | Set 4 | Positive- | | Test Set 4 | Positive- |
| Set 4 | Positive- | Necrosis with | Test Set 4 | Positive- | Necrosis with |
| Negative | Necrosis | Inflammation | Negative | Necrosis | Inflammation |
| AMPB-High | APAP-High | AFLB | 5-FU-High | CCL4-Low | ANIT-High |
| ANIT-Low | TET-High | BRB-High | 5-FU-Low | | LPS-High |
| AZA-High | | BRB-Low | AMPB-Low | | |
| AZA-Low | | CCL4-High | APAP-Low | | |
| BAP | | DMN | BEN-High | | |
| BEN-Low | | | CHLOR-Low | | |
| BUS | | | CIS-High | | |
| CAD-High | | | CIS-Low | | |
| CAD-Low | | | CLO-High | | |
| CAR | | | CPHOS-High | | |
| CHCL3-High | | | CYCA-High | | |
| CHCL3-Low | | | CYCA-Low | | |
| CHEX-High | | | ERY-High | | |
| CHEX-Low | | | ERY-Low | | |
| CHLOR-High | | | ISON-High | | |
| CLO-Low | | | ISON-Low | | |
| CLOZ-High | | | KETO-Low | | |
| CLOZ-Low | | | PBARB-Low | | |
| CMC | | | PHEN-Low | | |
| CPHOS-Low | | | QUIN-Low | | |
| DEX-High | | | TET-Low | | |
| DEX-Low | | | THEO-Low | | |
| DIF-High | | | | | |
| DIF-Low | | | | | |
| DOX | | | | | |
| EST-High | | | | | |
| EST-Low | | | | | |
| ETH | | | | | |
| GAN-High | | | | | |
| GAN-Low | | | | | |
| GEN-High | | | | | |
| GEN-Low | | | | | |
| HYD-High | | | | | |
| HYD-Low | | | | | |
| KETO-High | | | | | |
| LPS-Low | | | | | |
| MET | | | | | |
| NAL-High | | | | | |
| NAL-Low | | | | | |
| PBARB-High | | | | | |
| PEG | | | | | |
| PHEN-High | | | | | |
| PUR-High | | | | | |
| PUR-Low | | | | | |
| QUIN-High | | | | | |
| STRZ-High | | | | | |
| STRZ-Low | | | | | |
| TAM-High | | | | | |
| TAM-Low | | | | | |
| THEO-High | | | | | |

Training and Test Set 5

| | Training | Training Set 5 | | | Test Set 5 |
|---|---|---|---|---|---|
| Training | Set 5 | Positive- | | Test Set 5 | Positive- |
| Set 5 | Positive- | Necrosis with | Test Set 5 | Positive- | Necrosis with |
| Negative | Necrosis | Inflammation | Negative | Necrosis | Inflammation |
| TAM-Low | APAP-High | ANIT-High | AMPB-Low | TET-High | BRB-Low |
| CAR | CCL4-Low | BRB-High | ANIT-Low | | AFLB |
| 5-FU-High | | CCL4-High | AZA-Low | | |
| 5-FU-Low | | DMN | BEN-Low | | |
| AMPB-High | | LPS-High | CAD-Low | | |
| APAP-Low | | | CHCL3-Low | | |
| AZA-High | | | CHLOR-High | | |
| BAP | | | CIS-High | | |
| BEN-High | | | DEX-Low | | |
| BUS | | | DIF-High | | |
| CAD-High | | | EST-Low | | |
| CHCL3-High | | | GAN-High | | |
| CHEX-High | | | GAN-Low | | |
| CHEX-Low | | | GEN-High | | |
| CHLOR-Low | | | HYD-High | | |
| CIS-Low | | | ISON-High | | |
| CLO-High | | | KETO-High | | |
| CLO-Low | | | NAL-High | | |
| CLOZ-High | | | PBARB-Low | | |
| CLOZ-Low | | | STRZ-High | | |
| CMC | | | TET-Low | | |
| CPHOS-High | | | THEO-High | | |
| CPHOS-Low | | | | | |
| CYCA-High | | | | | |
| CYCA-Low | | | | | |
| DEX-High | | | | | |
| DIF-Low | | | | | |
| DOX | | | | | |
| ERY-High | | | | | |
| ERY-Low | | | | | |
| EST-High | | | | | |
| ETH | | | | | |
| GEN-Low | | | | | |
| HYD-Low | | | | | |
| ISON-Low | | | | | |
| KETO-Low | | | | | |
| LPS-Low | | | | | |
| ET | | | | | |
| NAL-Low | | | | | |
| PBARB-High | | | | | |
| PEG | | | | | |
| PHEN-High | | | | | |
| PHEN-Low | | | | | |
| PUR-High | | | | | |
| PUR-Low | | | | | |
| QUIN-High | | | | | |
| QUIN-Low | | | | | |
| STRZ-Low | | | | | |
| TAM-High | | | | | |
| THEO-Low | | | | | |

TABLE 21
List of Genes, Whose Expression at 72 h Directly Correlates
with Liver Inflammation at 72 h, Ranked by Pearson
Correlation Coefficient

| | Correlation |
|---|---|
| Gene | Coefficient |
| Osteoactivin | 0.780 |
| Calpactin I heavy chain | 0.719 |
| IgE binding protein | 0.686 |
| Thymosin beta-10 | 0.672 |
| Stathmin | 0.666 |
| Alpha-tubulin | 0.643 |
| Gamma-actin, cytoplasmic | 0.636 |
| 14-3-3 zeta | 0.630 |
| Phase-1 RCT-179 | 0.630 |
| High affinity IgE receptor gamma chain | 0.627 |
| (FcERIgamma) | |
| Uncoupling protein 2 | 0.626 |
| Voltage-dependent anion channel 2 (Vdac2) | 0.624 |
| Phase-1 RCT-154 | 0.622 |
| Melanoma-associated antigen ME491 | 0.619 |
| Phase-1 RCT-121 | 0.612 |
| Phase-1 RCT-138 | 0.600 |
| Phase-1 RCT-192 | 0.597 |
| Phase-1 RCT-68 | 0.587 |
| Phase-1 RCT-24 | 0.574 |
| Beta-tubulin, class I | 0.562 |
| Beta-actin | 0.550 |
| Beta-actin, sequence 2 | 0.549 |
| 60S ribosomal protein L6 | 0.549 |
| Cofilin | 0.549 |
| Pyruvate kinase, muscle | 0.547 |
| Phase-1 RCT-146 | 0.514 |
| Phase-1 RCT-207 | 0.513 |
| Organic cation transporter 3 | 0.506 |
| Phase-1 RCT-293 | 0.504 |
| Phase-1 RCT-12 | 0.502 |
| Phase-1 RCT-211 | 0.502 |
| Annexin V | 0.499 |
| Calpain 2 | 0.490 |
| Multidrug resistant protein-1 | 0.489 |
| Multidrug resistant protein-2 | 0.486 |
| Cathepsin S | 0.484 |
| Phase-1 RCT-144 | 0.484 |
| Cyclin D1 | 0.479 |
| 60S ribosomal protein L6 (alternate clone 1) | 0.479 |
| Biliverdin reductase | 0.477 |
| Nucleoside diphosphate kinase beta isoform | 0.477 |
| Collagen type II | 0.467 |
| Cyclin G | 0.458 |
| Cathepsin B | 0.454 |
| Phase-1 RCT-59 | 0.449 |
| Ribosomal protein S8 | 0.445 |
| Proliferating cell nuclear antigen gene | 0.442 |
| Phase-1 RCT-109 | 0.440 |
| Hypoxanthine-guanine | 0.438 |
| phosphoribosyltransferase | |
| Tissue inhibitor of metalloproteinases-1 | 0.435 |
| Poly(ADP-ribose) polymerase | 0.434 |
| Ribosomal protein S9 | 0.433 |
| Tissue plasminogen activator | 0.419 |
| Adenine nucleotide translocator 1 | 0.415 |
| Alpha-prothymosin | 0.409 |
| Ribosomal protein S17 | 0.407 |
| Heme oxygenase | 0.404 |
| p55CDC | 0.403 |
| ID-1 | 0.403 |
| Zinc finger protein | 0.401 |

TABLE 22
List of Genes, Whose Expression at 72 h Inversely
Correlates with Liver Inflammation at 72 h,
Ranked by Spearman Correlation Coefficient

| | Correlation |
|---|---|
| Gene | Coefficient |
| Phase-1 RCT-181 | −0.250 |
| Apolipoprotein C1 | −0.251 |
| Hepatic lipase | −0.253 |
| Tryptophan hydroxylase | −0.253 |
| Tissue factor | −0.254 |
| Monoamine oxidase B | −0.255 |
| Choline kinase | −0.256 |
| CDK108 | −0.257 |
| Phase-1 RCT-88 | −0.259 |
| Cholesterol esterase | −0.260 |
| Vesicular monoamine transporter (VMAT) | −0.260 |
| Glucokinase | −0.261 |
| Interferon inducible protein 10 | −0.264 |
| Cytochrome P450 2D18 | −0.264 |
| Aldehyde dehydrogenase 2 | −0.265 |
| Phase-1 RCT-93 | −0.265 |
| Connexin-32 | −0.267 |
| Phase-1 RCT-178 | −0.267 |
| Phase-1 RCT-239 | −0.268 |
| Phase-1 RCT-289 | −0.270 |
| C-reactive protein | −0.271 |
| Urinary protein 2 precursor | −0.273 |
| Matrin F/G | −0.274 |
| L-gulono-gamma-lactone oxidase | −0.276 |
| Epidermal growth factor | −0.278 |
| Tyrosine hydroxylase | −0.282 |
| Aquaporin-3 (AQP3) | −0.283 |
| Gap junction membrane channel protein beta 1 (Gjb1) | −0.283 |
| Phase-1 RCT-38 | −0.287 |
| NADH-cytochrome b5 reductase | −0.287 |
| Phase-1 RCT-256 | −0.288 |
| Phase-1 RCT-36 | −0.292 |
| Phase-1 RCT-271 | −0.293 |
| Acetylcholine receptor epsilon | −0.293 |
| Phase-1 RCT-73 | −0.293 |
| Phase-1 RCT-184 | −0.295 |
| Contrapsin-like protease inhibitor (CPi-21) | −0.297 |
| Phase-1 RCT-280 | −0.299 |
| Presenilin-1 | −0.300 |
| BRCA1 | −0.303 |
| Phase-1 RCT-219 | −0.305 |
| Cytochrome P450 2A3 | −0.306 |
| Phase-1 RCT-161 | −0.306 |
| Alpha 1 —inhibitor III | −0.307 |
| Cytochrome P450 3A1 | −0.307 |
| Carbonic anhydrase III | −0.308 |
| Aryl sulfotransferase | −0.308 |
| Acetyl-CoA carboxylase | −0.310 |
| Insulin-like growth factor I | −0.313 |
| Phase-1 RCT-67 | −0.313 |
| Protein tyrosine phosphatase, receptor type, D | −0.314 |
| Phase-1 RCT-285 | −0.315 |
| Phase-1 RCT-123 | −0.316 |
| Phase-1 RCT-98 | −0.317 |
| Arginosuccinate synthetase 1 | −0.319 |
| Phase-1 RCT-83 | −0.319 |
| Cytochrome P450 2C11 | −0.320 |
| Phase-1 RCT-149 | −0.320 |
| Phase-1 RCT-227 | −0.325 |
| Phase-1 RCT-102 | −0.330 |
| Phase-1 RCT-48 | −0.330 |
| Phase-1 RCT-29 | −0.331 |
| Betaine homocysteine methyltransferase (BHMT) | −0.335 |
| Stearyl-CoA desaturase, liver | −0.337 |
| Phase-1 RCT-292 | −0.337 |
| Apolipoprotein CIII | −0.339 |
| Fatty acid synthase | −0.340 |
| Phase-1 RCT-164 | −0.354 |
| Phase-1 RCT-81 | −0.354 |
| JNK1 stress activated protein kinase | −0.355 |
| Phase-1 RCT-260 | −0.355 |
| Equilbrative nitrobenzylthioinosine-sensitive nucleoside | −0.361 |
| transporter | |
| Phase-1 RCT-290 | −0.361 |
| Insulin-like growth factor I, exon 6 | −0.361 |
| Phase-1 RCT-117 | −0.363 |
| N-hydroxy-2-acetylaminofluorene sulfotransferase (ST1C1) | −0.363 |
| Glycine methyltransferase | −0.370 |
| Phase-1 RCT-107 | −0.378 |
| Apolipoprotein All | −0.381 |
| Dynamin-1 (D100) | −0.391 |
| Alpha-2-microglobulin | −0.395 |
| Phase-1 RCT-78 | −0.402 |

TABLE 23
List of genes whose expression at 72 hours is
predictive of liver inflammation at 72 hours

| | Combinations |
|---|---|
| | (No of |
| Gene | Occurrences) |
| Osteoactivin | 5 |
| Phase-1 RCT-211 | 5 |
| Calpactin I heavy chain | 5 |
| Phase-1 RCT-179 | 5 |
| Gamma-actin, cytoplasmic | 5 |
| Cofilin | 4 |
| Stathmin | 4 |
| 60S ribosomal protein L6 | 4 |
| Voltage-dependent anion channel 2 (Vdac2) | 4 |
| Phase-1 RCT-192 | 4 |
| Adenine nucleotide translocator 1 | 4 |
| Thymosin beta-10 | 4 |
| High affinity IgE receptor gamma chain (FcERIgamma) | 4 |
| Uncoupling protein 2 | 4 |
| IgE binding protein | 4 |
| Alpha-tubulin | 4 |
| Phase-1 RCT-12 | 4 |
| Ribosomal protein S9 | 4 |
| Phase-1 RCT-121 | 4 |
| 14-3-3 zeta | 4 |
| Beta-tubulin, class I | 4 |
| Phase-1 RCT-154 | 4 |
| Phase-1 RCT-107 | 3 |
| Proliferating cell nuclear antigen gene | 3 |
| Phase-1 RCT-59 | 3 |
| Beta-actin, sequence 2 | 3 |
| Phase-1 RCT-109 | 3 |
| Carbonic anhydrase III | 3 |
| Phase-1 RCT-78 | 3 |
| Collagen type II | 3 |
| Cyclin D1 | 3 |
| Phase-1 RCT-138 | 3 |
| Alpha-prothymosin | 3 |
| Calpain 2 | 3 |
| Cathepsin B | 3 |
| Phase-1 RCT-24 | 3 |
| Melanoma-associated antigen ME491 | 3 |
| Phase-1 RCT-68 | 3 |
| Cyclin G | 3 |
| Tissue inhibitor of metalloproteinases-1 | 3 |
| Heme oxygenase | 3 |
| Ribosomal protein S17 | 3 |
| Organic cation transporter 3 | 3 |
| Biliverdin reductase | 3 |
| Phase-1 RCT-293 | 3 |
| Phase-1 RCT-173 | 3 |
| Betaine homocysteine methyltransferase (BHMT) | 2 |
| Cytochrome P450 2D18 | 2 |
| Cytochrome P450 2C11 | 2 |
| Phase-1 RCT-290 | 2 |
| Pyruvate kinase, muscle | 2 |
| Apolipoprotein All | 2 |
| Connexin-32 | 2 |
| Glycine methyltransferase | 2 |
| Insulin-like growth factor I | 2 |
| Zinc finger protein | 2 |
| Hypoxanthine-guanine phosphoribosyltransferase | 2 |
| ID-1 | 2 |
| Ribosomal protein S8 | 2 |
| Nucleoside diphosphate kinase beta isoform | 2 |
| 60S ribosomal protein L6 (alternate clone 1) | 2 |
| Beta-actin | 2 |
| Cathepsin S | 2 |
| Annexin V | 2 |
| Phase-1 RCT-276 | 2 |
| Tyrosine aminotransferase | 2 |
| Phase-1 RCT-161 | 2 |
| Multidrug resistant protein-2 | 2 |
| DNA polymerase beta | 2 |
| Ubiquitin conjugating enzyme (RAD 6 homologue) | 2 |
| Ribosomal protein L13A | 2 |
| Phase-1 RCT-144 | 2 |
| c-H-ras | 2 |
| Vesicular monoamine transporter (VMAT) | 2 |
| Phase-1 RCT-273 | 2 |
| Phase-1 RCT-80 | 2 |
| Phase-1 RCT-260 | 2 |
| Neuronal cell adhesion molecule (NrCAM) | 2 |
| Hepatocyte growth factor receptor | 2 |
| Caveolin-3 | 2 |
| Phase-1 RCT-129 | 2 |
| Phase-1 RCT-146 | 2 |
| Phase-1 RCT-292 | 1 |
| L-gulono-gamma-lactone oxidase | 1 |
| Phase-1 RCT-256 | 1 |
| Urinary protein 2 precursor | 1 |
| Aryl sulfotransferase | 1 |
| Phase-1 RCT-185 | 1 |
| Phase-1 RCT-34 | 1 |
| Phase-1 RCT-31 | 1 |
| Complement factor I (CFI) | 1 |
| Glutathione peroxidase | 1 |
| Histidine-rich glycoprotein | 1 |
| Carbonic anhydrase III, sequence 2 | 1 |
| Phase-1 RCT-92 | 1 |
| Transitional endoplasmic reticulum ATPase | 1 |
| Phase-1 RCT-88 | 1 |
| Phase-1 RCT-296 | 1 |
| Glutathione S-transferase theta-1 | 1 |
| Phase-1 RCT-168 | 1 |
| Phase-1 RCT-182 | 1 |
| JNK1 stress activated protein kinase | 1 |
| Phase-1 RCT-81 | 1 |
| Phase-1 RCT-33 | 1 |
| Phase-1 RCT-178 | 1 |
| Apolipoprotein CIII | 1 |
| Phase-1 RCT-98 | 1 |
| NADH-cytochrome b5 reductase | 1 |
| Alpha 1 —inhibitor III | 1 |
| Phase-1 RCT-233 | 1 |
| Paraoxonase 1 | 1 |
| Presenilin-1 | 1 |
| Apolipoprotein C1 | 1 |
| Cytochrome P450 2C23 | 1 |
| Phase-1 RCT-227 | 1 |
| Hepatic lipase | 1 |
| Phase-1 RCT-164 | 1 |
| Insulin-like growth factor I, exon 6 | 1 |
| N-hydroxy-2-acetylaminofluorene sulfotransferase | 1 |
| (ST1C1) | |
| Dynamin-1 (D100) | 1 |
| Phase-1 RCT-230 | 1 |
| Phase-1 RCT-74 | 1 |
| Phase-1 RCT-158 | 1 |
| Deoxycytidine kinase | 1 |
| Dopamine receptor D2 | 1 |
| Phase-1 RCT-51 | 1 |
| Four repeat ion channel | 1 |
| Adrenomedullin | 1 |
| Phase-1 RCT-94 | 1 |
| Sarcoplasmic reticulum calcium ATPase | 1 |
| Phase-1 RCT-79 | 1 |
| Phase-1 RCT-252 | 1 |
| Phase-1 RCT-151 | 1 |
| Phase-1 RCT-70 | 1 |
| Phase-1 RCT-150 | 1 |
| 25-hydroxyvitamin D3-1 alpha-hydroxylase | 1 |
| Phase-1 RCT-119 | 1 |
| Peroxisomal 3-ketoacyl-CoA thiolase 2 | 1 |
| Superoxide dismutase Mn | 1 |
| Phase-1 RCT-115 | 1 |
| Alpha-1 microglobulin/bikunin precursor (Ambp) | 1 |
| Phase-1 RCT-18 | 1 |
| Maspin | 1 |
| Decorin | 1 |
| Retinoid X receptor alpha | 1 |
| Cellular nucleic acid binding protein (CNBP) | 1 |
| NADPH cytochrome P450 oxidoreductase | 1 |
| Malic enzyme | 1 |
| Caspase 1 | 1 |
| Cystatin C | 1 |
| p55CDC | 1 |
| Poly(ADP-ribose) polymerase | 1 |
| Tissue plasminogen activator | 1 |
| Multidrug resistant protein-1 | 1 |
| Phase-1 RCT-207 | 1 |
| Phase-1 RCT-181 | 1 |
| Gap junction membrane channel protein beta 1 (Gjb1) | 1 |
| Aquaporin-3 (AQP3) | 1 |
| Myelin basic protein | 1 |
| Phase-1 RCT-213 | 1 |
| Phase-1 RCT-156 | 1 |
| Proteasome activator 28 alpha | 1 |

TABLE 24
Comparison of Predictivity for True Liver Inflammation Classification
and Random Classification Using Combo Gene Sets and 72 h data

| | Overall Accuracy** | | | |
|---|---|---|---|---|
| | Correct Classification | | Random Classification | |
| Gene List* | Mean | Min-Max | Mean | Min.-Max. |
| Combo All | 0.752 | (0.625-0.847) | 0.368 | (0.250-0.459) |
| Combo 5 | 0.672 | (0.589-0.722) | 0.363 | (0.295-0.419) |
| Combo 4 | 0.793 | (0.694-0.917) | 0.344 | (0.222-0.458) |
| Combo 3 | 0.793 | (0.639-0.905) | 0.333 | (0.250-0.392) |
| Combo 2 | 0.708 | (0.597-0.819) | 0.349 | (0.288-0.473) |
| Combo 1 | 0.675 | (0.608-0.708) | 0.377 | (0.208-0.466) |

TABLE 25
RCT genes (ESTs) Predictive for Liver Inflammation:
Best Homology Matches

| Gene Name | Homology |
|---|---|
| Phase-1 RCT-10 | Rattus norvegicus methylmalonate semialdehyde dehydrogenase gene |
| | (Mmsdh) |
| Phase-1 RCT-102 | Mouse pentylenetetrazol-related mRNA PTZ-17 (3′UTR of E3.1) |
| Phase-1 RCT-103 | no significant homology found |
| Phase-1 RCT-107 | no significant homology found |
| Phase-1 RCT-108 | no significant homology found |
| Phase-1 RCT-109 | Rattus norvegicus nesprin-1 mRNA |
| Phase-1 RCT-111 | Mus musculus B lymphoid kinase (Blk) |
| Phase-1 RCT-112 | no significant homology found |
| Phase-1 RCT-113 | no significant homology found |
| Phase-1 RCT-114 | Mus musculus, glypican 4, clone MGC:11506 IMAGE:3967797, mRNA, |
| | complete cds |
| Phase-1 RCT-115 | no significant homology found |
| Phase-1 RCT-117 | no significant homology found |
| Phase-1 RCT-119 | no significant homology found |
| Phase-1 RCT-12 | no significant homology found |
| Phase-1 RCT-121 | no significant homology found |
| Phase-1 RCT-123 | no significant homology found |
| Phase-1 RCT-127 | no significant homology found |
| Phase-1 RCT-128 | Mus musculus angiopoietin-related protein 3 (Angpt13) |
| Phase-1 RCT-129 | Mus musculus Nedd4 WW binding protein 4 (N4wbp4-pending), mRNA |
| Phase-1 RCT-13 | Mus musculus 0 day neonate skin cDNA, RIKEN full-length enriched |
| | library, clone:4632417K18, full insert sequence |
| Phase-1 RCT-136 | Mus musculus RIKEN cDNA 3010027G13 gene (3010027G13Rik), |
| | mRNA |
| Phase-1 RCT-137 | Mus musculus adult male tongue cDNA |
| Phase-1 RCT-138 | Mus musculus DAP10 (Dap10) gene |
| Phase-1 RCT-140 | Mouse 13 days embryo head cDNA, RIKEN full-length enriched library, |
| | clone:3100001I08 |
| Phase-1 RCT-141 | Mus musculus proteoglycan 3 (megakaryocyte stimulating factor, |
| | articular superficial zone protein) (Prg4) |
| Phase-1 RCT-142 | Mus musculus 18 days embryo cDNA, RIKEN full-length enriched library, |
| | clone:1190008J14 |
| Phase-1 RCT-143 | Homo sapiens NADH dehydrogenase (ubiquinone) Fe—S protein 8 (23 kD) |
| | (NADH-coenzyme Q reductase) (NDUFS8) |
| Phase-1 RCT-144 | Mus musculus, similar to nucleolar protein (KKE/D repeat), clone |
| | IMAGE:3491448, mRNA, partial cds. |
| Phase-1 RCT-145 | Mus musculus 10 day old male pancreas cDNA, RIKEN full-length |
| | enriched library, clone:1810014B19, full insert sequence |
| Phase-1 RCT-146 | Mus musculus 8 days embryo cDNA, RIKEN full-length enriched library, |
| | clone:5730458E20 |
| Phase-1 RCT-148 | Mus musculus adult male kidney cDNA, RIKEN full-length enriched |
| | library, clone:0610010B16 |
| Phase-1 RCT-15 | Mus musculus ubiquitin conjugating enzyme 7 mRNA, complete cds |
| Phase-1 RCT-150 | Mus musculus SIR2L3 isoform B (Sir2L3) mRNA, complete |
| | cds;alternatively spliced |
| Phase-1 RCT-151 | Mus musculus, Similar to sphingomyelin phosphodiesterase 1, acid |
| | lysosomal, clone MGC:11522 IMAGE:3964394 |
| Phase-1 RCT-152 | Mus musculus, eukaryotic translation elongation factor 1 beta 2, clone |
| | MGC:6763 IMAGE:3600850, mRNA, complete cds. |
| Phase-1 RCT-154 | Mus musculus vacuolar ATPase subunit D (Atp6m) mRNA, complete cds |
| Phase-1 RCT-156 | no significant homology found |
| Phase-1 RCT-158 | Rattus norvegicus cyclin-dependent kinase inhibitor 1B |
| Phase-1 RCT-161 | Mus musculus adult male spleen cDNA, RIKEN full-length enriched |
| | library, clone:0910001D19 |
| Phase-1 RCT-164 | Mus musculus adult male testis cDNA, RIKEN full-length enriched |
| | library, clone:4932443D16 |
| Phase-1 RCT-166 | Mus musculus, Similar to glutathione S-transferase theta 1, clone |
| | MGC:6769 IMAGE:3601446 |
| Phase-1 RCT-168 | M. musculus mRNA for low density lipoprotein receptor, ACCESSION |
| | X64414 S51850 |
| Phase-1 RCT-169 | Mus musculus, small inducible cytokine B subfamily (Cys-X-Cys), |
| | member 9, clone MGC:6179 IMAGE:3257716, mRNA, complete |
| Phase-1 RCT-173 | Mus musculus NADP + -specific isocitrate dehydrogenase mRNA, |
| | complete cds; nuclear gene for mitochondrial product |
| Phase-1 RCT-174 | Homo sapiens normal mucosa of esophagus specific 1 (NMES1) mRNA, |
| | complete cds; nuclear gene for mitochondrial product |
| Phase-1 RCT-174 | Mus musculus RIKEN cDNA 1190017B19 gene (1190017B19Rik), |
| | mRNA, |
| Phase-1 RCT-178 | Mus musculus, thioether S-methyltransferase, clone MGC:19191 |
| | IMAGE:4236077, mRNA, complete cds |
| Phase-1 RCT-179 | Rat nucleolar protein B23.2 mRNA |
| Phase-1 RCT-18 | no significant homology found |
| Phase-1 RCT-180 | Mus musculus B-cell receptor-associated protein 37 (Bcap37 |
| Phase-1 RCT-181 | Mus musculus adult male testis cDNA |
| Phase-1 RCT-182 | Rattus norvegicus glb mRNA for diacetyl/L-xylulose reductase |
| Phase-1 RCT-184 | no significant homology found |
| Phase-1 RCT-185 | no significant homology found |
| Phase-1 RCT-189 | Rattus norvegicus eukaryotic translation initiation factor 4E (Eif4e), |
| | mRNA |
| Phase-1 RCT-191 | Mus musculus, Similar to proteasome (prosome, macropain) 26S |
| | subunit, non-ATPase, 3, clone MGC:6405 IMAGE:3586427, mRNA, |
| | complete cds |
| Phase-1 RCT-192 | Mus musculus 18 days embryo cDNA, RIKEN full-length enriched library, |
| | clone:1110033J19 |
| Phase-1 RCT-195 | Mus musculus, Similar to protein kinase C substrate 80K-H, clone |
| | MGC:13908 IMAGE:4008182, mRNA, complete cds |
| Phase-1 RCT-196 | Homolous to Mus musculus 12 days embryo head cDNA, RIKEN full- |
| | length enriched library, clone:3010001M15 |
| Phase-1 RCT-197 | Rattus norvegicus Protein kinase, interferon-inducible double stranded |
| | RNA dependent (Prkr), mRNA |
| Phase-1 RCT-202 | Mus musculus, Similar to hypothetical protein AB030201, clone |
| | MGC:18837 IMAGE:4211629, mRNA, complete cds |
| Phase-1 RCT-204 | Mouse DNA sequence from clone RP23-138F20 on chromosome 13, |
| | complete sequence [Mus musculus] |
| Phase-1 RCT-205 | no significant homology found |
| Phase-1 RCT-207 | Mus musculus Ran binding protein 5 mRNA, partial cds |
| Phase-1 RCT-209 | Mus musculus adult male testis cDNA, RIKEN full-length enriched |
| | library, clone:4930583H14, full insert sequence |
| Phase-1 RCT-211 | Mus musculus adult male kidney cDNA, RIKEN full-length enriched |
| | library, clone:0610009C22 |
| Phase-1 RCT-212 | Mus musculus nuclear localization signal protein absent in velo-cardio- |
| | facial patients (Nlvcf) |
| Phase-1 RCT-213 | Homo sapiens pM5 protein (PM5), mRNA |
| Phase-1 RCT-214 | Mus musculus putative AND(P)H steroid dehydrogenase mRNA |
| Phase-1 RCT-215 | Mus musculus RAB/Rip protein mRNA |
| Phase-1 RCT-218 | no significant homology found |
| Phase-1 RCT-219 | Rattus norvegicus 2′5′ oligoadenylate synthetase-2 mRNA, complete cds |
| Phase-1 RCT-22 | Mus musculus, clone MGC:19042 IMAGE:4188988, mRNA |
| Phase-1 RCT-221 | no significant homology found |
| Phase-1 RCT-225 | Rattus norvegicus chromosome 4 clone RP31-327J16 strain Brown |
| | Norway, complete sequence |
| Phase-1 RCT-227 | no significant homology found |
| Phase-1 RCT-230 | Mus musculus GDP-dissociation inhibitor mRNA, preferentially |
| | expressed in hematopoietic cells, complete cds |
| Phase-1 RCT-233 | no significant homology found |
| Phase-1 RCT-235 | Rattus villosissimus RT1.Ba gene, RT1.Ba-R154 allele, intron b, |
| | complete sequence |
| Phase-1 RCT-239 | Mus musculus adult male tongue cDNA, RIKEN full-length enriched |
| | library, clone:2300007B01, full insert sequence |
| Phase-1 RCT-24 | Mus musculus, tubulin alpha 8, clone MGC:28850 IMAGE:4507364, |
| | mRNA, |
| Phase-1 RCT-240 | Mus musculus, clone MGC:7041 |
| Phase-1 RCT-241 | Mus musculus oncostatin receptor (Osmr), mRNA |
| Phase-1 RCT-242 | Rattus norvegicus B-cell translocation gene 2, anti-proliferative(Btg2), |
| Phase-1 RCT-25 | Mouse DNA sequence from clone RP23-278F12 on chromosome 11, |
| | complete sequence |
| Phase-1 RCT-251 | no significant homology found |
| Phase-1 RCT-252 | Mus musculus EH-domain containing 3 (Ehd3), |
| Phase-1 RCT-256 | Mus musculus, Similar to betaine-homocysteine methyltransferase 2, |
| | clone MGC:19186 IMAGE:4235455 |
| Phase-1 RCT-258 | Mus musculus, clone MGC:6139 IMAGE:3487295, mRNA |
| Phase-1 RCT-259 | Mus musculus adult female placenta cDNA, RIKEN full-length enriched |
| | library, clone:1600023I01:interferon-stimulated protein (20 kDa), full |
| | insert sequence |
| Phase-1 RCT-260 | Mus musculus adult male hippocampus cDNA, RIKEN full-length |
| | enriched library, clone:2900024P20 |
| Phase-1 RCT-261 | no significant homology found |
| Phase-1 RCT-264 | Mus musculus sodium-sulfate cotransporter (Nas1) gene |
| Phase-1 RCT-27 | Mus musculus adult male kidney cDNA |
| Phase-1 RCT-270 | Mus musculus, RIKEN cDNA 2010011I20 gene, clone MGC:27703, |
| | IMAGE:4924329, mRNA, complete cds |
| Phase-1 RCT-271 | Homlogous to Mus musculus, clone MGC:27581 IMAGE:4489072, |
| | mRNA |
| Phase-1 RCT-273 | no significant homology found |
| Phase-1 RCT-276 | Homo sapiens KIAA1224 protein |
| Phase-1 RCT-278 | Mus musculus brain protein 17 (Brp17), mRNA |
| Phase-1 RCT-28 | no significant homology found |
| Phase-1 RCT-280 | Mus musculus carbohydrate (keratan sulfate Gal-6) sulfotransferase 1 |
| | (Chst1), |
| Phase-1 RCT-281 | Mus musculus, Similar to TNF-induced protein, clone MGC:11714 |
| Phase-1 RCT-282 | Mus musculus, SEC61, alpha subunit 2 (S. cerevisiae), clone MGC:6359 |
| | IMAGE:3494001, mRNA, complete cds |
| Phase-1 RCT-287 | Mus musculus adult male kidney cDNA clone:0610010I20 |
| Phase-1 RCT-288 | no significant homology found |
| Phase-1 RCT-289 | Mus musculus adult male liver cDNA, RIKEN full-length enriched library, |
| | clone:1300003K24, full insert sequence |
| Phase-1 RCT-29 | no significant homology found |
| Phase-1 RCT-290 | Homo sapiens chromosome 14 clone BAC 201F1 map 14q24.3, |
| | complete sequence |
| Phase-1 RCT-291 | no significant homology found |
| Phase-1 RCT-292 | Rattus norvegicus 2′5′ oligoadenylate synthetase-2 |
| Phase-1 RCT-293 | Mus musculus 18 days embryo cDNA, RIKEN full-length enriched library, |
| | clone:1110021C22 |
| Phase-1 RCT-294 | Mus musculus adult male cerebellum cDNA, RIKEN full-length enriched |
| | library, clone:1500035D08:vesicle-associated membrane protein 1, full |
| | insert sequence |
| Phase-1 RCT-296 | Mus musculus corticosteroid binding globulin (Cbg) |
| Phase-1 RCT-297 | Mus musculus squalene epoxidase (Sqle), H |
| Phase-1 RCT-3 | no significant homology found |
| Phase-1 RCT-30 | Homo sapiens putative protein-tyrosine kinase (LOC51086), |
| Phase-1 RCT-31 | Mouse 10, 11 days embryo cDNA, RIKEN full-length enriched library, |
| | clone:2810437P06 |
| Phase-1 RCT-32 | no significant homology found |
| Phase-1 RCT-33 | no significant homology found |
| Phase-1 RCT-34 | no significant homology found |
| Phase-1 RCT-36 | no significant homology found |
| Phase-1 RCT-37 | no significant homology found |
| Phase-1 RCT-38 | Mus musculus betaine-homocysteine methyltransferase 2 (Bhmt2) |
| | mRNA, |
| Phase-1 RCT-40 | Rattus norvegicus Cathepsin C (dipeptidyl peptidase I) (Ctsc) |
| Phase-1 RCT-42 | Mus musculus STAT5B (Stat5b) |
| Phase-1 RCT-43 | no significant homology found |
| Phase-1 RCT-45 | Mus musculus Nedd4-binding brain specific protein BEAN mRNA, partial |
| | cds |
| Phase-1 RCT-48 | Mus musculus adult male liver cDNA, RIKEN full-length enriched library, |
| | clone:1300003K24, full insert sequence |
| Phase-1 RCT-49 | No match with score above 200 |
| Phase-1 RCT-50 | Mus musculus fibroblast growth factor regulated protein 2 |
| Phase-1 RCT-51 | Rattus norvegicus unknown Glu-Pro dipeptide repeat protein |
| Phase-1 RCT-52 | Rattus norvegicus D5d mRNA for delta-5 fatty acid desaturase |
| Phase-1 RCT-53 | no significant homology found |
| Phase-1 RCT-54 | Mus musculus 10 days embryo cDNA, RIKEN full-length enriched library, |
| | clone:2610007A05, full insert sequence |
| Phase-1 RCT-55 | M. musculus myoglobin gene exons 2-3 |
| Phase-1 RCT-56 | M. musculus myoglobin gene exons 2-3 |
| Phase-1 RCT-59 | no significant homology found |
| Phase-1 RCT-60 | Mouse, Similar to tyrosyl-tRNA synthetase, clone MGC:19350 |
| Phase-1 RCT-62 | no significant homology found |
| Phase-1 RCT-63 | no significant homology found |
| Phase-1 RCT-64 | no significant homology found |
| Phase-1 RCT-65 | no significant homology found |
| Phase-1 RCT-66 | M. musculus mRNA for low density lipoprotein receptor |
| Phase-1 RCT-67 | no significant homology found |
| Phase-1 RCT-68 | Rattus norvegicus nucleosome assembly protein mRNA |
| Phase-1 RCT-70 | Mus musculus adult male testis cDNA, RIKEN full-length enriched library, |
| | clone:4933406P04, full insert sequence |
| Phase-1 RCT-71 | Mus musculus, clone MGC:11987 IMAGE:3601737, mRNA |
| Phase-1 RCT-72 | no significant homology found |
| Phase-1 RCT-73 | no significant homology found |
| Phase-1 RCT-74 | no significant homology found |
| Phase-1 RCT-75 | Mus musculus adult male liver cDNA, RIKEN full-length enriched library, |
| | clone:1300002K09, full insert sequence |
| Phase-1 RCT-76 | no significant homology found |
| Phase-1 RCT-77 | Mus musculus, Similar to hypothetical protein AB030201, clone |
| | MGC:18837 IMAGE:4211629, mRNA, complete cds |
| Phase-1 RCT-78 | Mus musculus adult male lung cDNA, RIKEN full-length enriched library, |
| | clone:1200015G06, full insert sequence |
| Phase-1 RCT-79 | no significant homology found |
| Phase-1 RCT-8 | Messenger RNA for rat preproalbumin |
| Phase-1 RCT-80 | no significant homology found |
| Phase-1 RCT-81 | no significant homology found |
| Phase-1 RCT-82 | Mus musculus nucleosome binding protein 1 (Nsbp1), |
| Phase-1 RCT-83 | no significant homology found |
| Phase-1 RCT-88 | no significant homology found |
| Phase-1 RCT-89 | no significant homology found |
| Phase-1 RCT-9 | Mus musculus adult male liver cDNA, RIKEN full-length enriched library, |
| | clone:1300003M23, full insert sequence |
| Phase-1 RCT-90 | no significant homology found |
| Phase-1 RCT-91 | no significant homology found |
| Phase-1 RCT-92 | no significant homology found |
| Phase-1 RCT-94 | Rattus norvegicus Glutamate receptor, metabotropic 5 (Grm5) |
| Phase-1 RCT-95 | no significant homology found |
| Phase-1 RCT-96 | Mus musculus, ADP-ribosylation factor 3, clone MGC:6687 |
| | IMAGE:3582243, mRNA, complete cds, |

TABLE 27
Liver Inflammation Predictive Genes Whose
Protein Products Are Known to be Secreted

Adrenomedullin
Alpha 1 - inhibitor III
Alpha-1 acid glycoprotein
Alpha-1 microglobulin/bikunin precursor (Ambp)
Alpha-2-macroglobulin, sequence 2
Alpha-2-microglobulin
Alpha-fetoprotein
Apolipoprotein AII
Apolipoprotein C1
Apolipoprotein CIII
Apolipoprotein E
Ceruloplasmin
Ciliary neurotrophic factor
Colony-stimulating factor-1
Complement component C3
Complement factor I (CFI)
Histidine-rich glycoprotein
Insulin-like growth factor binding protein 1
Insulin-like growth factor binding protein 5
Insulin-like growth factor I
Insulin-like growth factor I, exon 6
Inter-alpha-inhibitor H4 heavy chain (Itih4)
Interferon related developmental regulator IFRD1 (PC4)
Interleukin-10
Macrophage inflammatory protein-1 alpha
Macrophage inflammatory protein-2 alpha
Matrix metalloproteinase-1
NGF-inducible anti-proliferative putative secreted protein
(PC3)
Osteopontin
Paraoxonase 1
Preproalbumin, sequence 2
Selenoprotein P
Stem cell factor
Tissue factor pathway inhibitor
Tissue inhibitor of metalloproteinases-1
Tissue plasminogen activator
Transthyretin
Urinary protein 2 precursor
Vascular endothelial growth factor

What is claimed is:

1. A method of predicting the liver toxicity in an individual to an agent comprising:
obtaining a biological sample from the individual treated with the agent;
measuring the expression of one or more liver toxicity predictive genes in the sample, wherein the genes are selected from the group consisting of partial gene sequences of genes identified as responsive to agents causing liver inflammation, thereby generating a test expression profile; and
using the test expression profile with a set of reference expression profiles in a Predictive Model to determine whether the agent will induce liver toxicity in the individual.

2. The method according to claim 1, wherein the liver toxicity predictive genes are selected from the group of partial gene sequences listed in Table26 that represent 24 hour combo AII genes.

3. The method according to claim 2, wherein the partial gene sequences correspond to rat genes.

4. The method according to claim. 2, wherein the partial gene sequences correspond to dog genes.

5. The method according to claim 2, wherein the partial gene sequences correspond to non-human primate genes.

6. The method according to claim 2, wherein the partial gene sequences correspond to human genes.

7. The method according to claim 1, wherein the liver toxicity predictive genes are selected from the group of partial gene sequences listed in Table26 that represent 24 hour combo 3 genes.

8. The method according to claim 7, wherein the partial gene sequences correspond to rat genes.

9. The method according to claim 7, wherein the partial gene sequences correspond to dog genes.

10. The method according to claim 7, wherein the partial gene sequences correspond to non-human primate genes.

11. The method according to claim 7, wherein the partial gene sequences correspond to human genes.

12. The method according to claim 1, wherein the liver toxicity predictive genes are selected from the group of partial gene sequences listed in Table 26 that represent 24 hour Combo 5 genes.

13. The method according to claim 12, wherein the partial gene sequences correspond to rat genes.

14. The method according to claim 12, wherein the partial gene sequences correspond to dog genes.

15. The method according to claim 12, wherein the partial gene sequences correspond to non-human primate genes.

16. The method according to claim 12, wherein the partial gene sequences correspond to human genes.

17. A method of predicting the liver toxicity of an agent using an in vitro system, comprising the steps of:
obtaining a biological sample from in-vitro cultured cells or explants treated with the agent;
measuring the expression of one or more liver toxicity predictive genes in the sample, wherein the genes are selected from the group consisting of partial gene sequences of genes identified as responsive to agents causing liver inflammation, thereby generating a test expression profile; and
using the test expression profile with a set of reference expression profiles in a Predictive Model to determine whether the agent will induce liver toxicity in the individual.

18. The method according to claim 17, wherein the liver toxicity predictive genes are selected from the group of partial gene sequences listed in Table 26 that represent 24 hour combo AII genes.

19. The method according to claim 18, wherein the partial gene sequences correspond to rat genes.

20. The method according to claim 18, wherein the partial gene sequences correspond to dog genes.

21. The method according to claim 18, wherein the partial gene sequences correspond to non-human primate genes.

22. The method according to claim 18, wherein the partial gene sequences correspond to human genes.

23. The method according to claim 17, wherein the liver toxicity predictive genes are selected from the group comprising of 24 hour Combo 2 genes.

24. The method according to claim 23, wherein the partial gene sequences correspond to rat genes.

25. The method according to claim 23, wherein the partial gene sequences correspond to dog genes.

26. The method according to claim 23, wherein the partial gene sequences correspond to non-human primate genes.

27. The method according to claim 23, wherein the partial gene sequences correspond to human genes.

28. The method according to claim 17, wherein the liver toxicity predictive genes are selected from the group of partial gene sequences listed in Table 26 that represent 24 hour Combo 5 genes.

29. The method according to claim 28, wherein the partial gene sequences correspond to rat genes.

30. The method according to claim 28, wherein the partial gene sequences correspond to dog genes.

31. The method according to claim 28, wherein the partial gene sequences correspond to non-human primate genes.

32. The method according to claim 28, wherein the partial gene sequences correspond to human genes.

33. A process for predicting the liver toxicity in a biological sample from an individual, in-vitro cell cultures or explants to an agent via a programmable machine, the process comprising the steps of:
obtaining a biological sample treated with the agent;
measuring the expression of one or more liver toxicity predictive genes in the sample, wherein the genes are selected from the group consisting of partial gene sequences of genes identified as responsive to agents causing liver inflammation, thereby generating a test expression profile; and
using the test expression profile with a set of reference expression profiles in a Predictive Model to-determine whether the agent will induce liver toxicity in the individual.

34. A computer program product for enabling a computer to perform Predictive Model analysis for liver toxicity on a biological sample from an individual, in-vitro cell cultures or explants to an agent, the computer program product comprising:
software instructions for enabling the computer to perform predetermined operations, and a computer readable medium embodying the software instructions;
the pre-determined operations comprising:
measuring an expression of one or more liver toxicity predictive genes in a sample, wherein the genes are selected from the group consisting of partial gene sequences of genes identified as responsive to agents causing liver inflammation, thereby generating a test expression profile; and
using the test expression profile with a set of reference expression profiles in a Predictive Model to determine whether the agent will induce liver toxicity in the individual.

35. A Computer system adopted to predict liver toxicity in a biological sample from an individual, in-vitro cell cultures, or explants to an agent, comprising a processor and a memory including software instructions adapted to enable the computer system to perform operations comprising:
measuring the expression of one or more liver toxicity predictive genes in the sample, wherein the genes are selected from the group consisting of partial gene sequences of genes identified as responsive to agents causing liver inflammation, thereby generating a test expression profile; and
using the test expression profile with a set of reference expression profiles in a Predictive Model to determine whether the agent will induce liver toxicity in the individual.

36. A computer program product for predicting liver toxicity from a test sample expression profile, comprising:
an encrypted training data set;
encrypted lists of genes selected from genes predictive of liver toxicity to be used with the encrypted training data set, and
a Predictive Model that uses the encrypted training data sets, the encrypted lists of genes, and the test sample expression profile to predict the liver toxicity of the test sample.

37. The computer program product of claim 36, wherein the encrypted lists of genes are selected from any Combination Category appearing in Tables 5, 18 and 23.

38. The computer program product of claim 36, wherein the encrypted lists of genes comprise a 24 hour Combo AII genes as set in Table 5.

39. The computer program product of claim 36, wherein the encrypted lists of genes comprise a 6 hour Combo AII genes as set in Table 18.

40. The computer program product of claim 36, wherein the encrypted lists of genes comprise a 72 hour Combo AII genes as set in Table 23.

41. A method for mining genes predictive for liver toxicity, comprising the steps of:
collecting expression levels of a plurality of candidate toxicity predictive genes among a multiplicity of samples;
defining a group of samples to be a training set;
defining another group of samples to be a test set;
optionally generating additional training and test sets; and
selecting a set of genes which are predictive of liver toxicity based on evaluating the training and test sets in a Predictive Model.

42. The method according to claim 41, wherein the expression levels are stored as a database on an electronic medium.

43. An integrated system for predicting liver toxicity, comprising:
means for measuring gene expression profiles of genes predictive of liver toxicity from biological samples exposed to a test agent; and
a computer system operably linked to the means wherein the computer system is capable of implementing a Predictive Model.

44. A method of identifying one or more liver inflammation predictive genes, the method comprising:
providing a set of candidate toxicity predictive genes;
evaluating said genes for their predictive performance with at least one training and test set of data in a Predictive Model to identify genes which are predictive of liver inflammation; and
testing the performance of predictive genes for their ability to predict liver inflammation for: (i) different test sets of data, (ii) comparison of prediction for accurate versus random classification, and (iii) prediction using test data external to the data used to derive the predictive genes.
